(19) United States
(12) Patent Application Publication
(10) Pub. No.: US 2002/0045610 A1
(43) Pub. Date: Apr. 18, 2002

(54) Use of N-substituted azaheterocyclic compounds for the manufacture of a pharmaceutical composition for the treatment of indications related to angiogenesis

(76) Inventors: Anker Jon Hansen, Charlottenlund (DK); Tine Krogh Jorgensen, Olstykke (DK); Uffe Bang Olsen, Vallensbaek (DK)

Correspondence Address: Reza Green, Esq., Novo Nordisk of North America, Inc., Suite 6400, 405 Lexington Avenue, New York, NY, 10174-6401, US

(21) Appl. No.: 09/872,127

(22) Filed: Jun. 1, 2001

Related U.S. Application Data
(63) Continuation of application No. PCT/DK99/00671, filed on Dec. 1, 1999.
(60) Provisional application No. 60/111,445, filed on Dec. 8, 1998.

(30) Foreign Application Priority Data
Dec. 2, 1998 (DK) PA 1998 01586

(51) Int. Cl. A61K 31/554; A61K 31/553; A61K 31/55

(52) U.S. Cl. 514/183; 514/211.11; 514/221; 514/217

(57) ABSTRACT

The present invention relates to the use of N-substituted azaheterocyclic compounds or salts thereof, for the treatment of conditions related to angiogenesis.

### FIELD OF INVENTION

The present invention relates to the use of N-substituted azaheterocyclic compounds of the general formulas Ia-Id for the treatment, prevention, alleviation or amelioration of conditions related to angiogenesis. Hence the compounds can be used in the treatment of patients suffering from a variety of diseases like abnormal tissue growth, neoplasia, hyperplasia, cancer, diabetic retinopathy. The present invention also embraces pharmaceutical compositions comprising those compounds and methods of using the compounds and their pharmaceutical compositions.

### BACKGROUND OF INVENTION

Tissue growth is critically dependent upon the formation of new capillaries, called angiogenesis or neovacularisation. The process may in pathological conditions be turned on by growth factors, e.g. vascular endothelial growth factor or cytokines, e.g. tumor necosis factor α. In e.g. cancer, angiogenesis is an important factor for the maintenance and growth of the tumor (Tanaka et al., Cancer Res., 58, 3362-3369, 1998). Angiogenesis is important for neoplastic conditions like cancer as well as ocular neovascularization like diabetic retinopathy (Favard et al., Diabetes and Metabolism 22, 268-273,1996). Thus it has been shown that treatments directed against angiogenesis can e.g. inhibit tumor growth (Folkman, J., Breast Cancer Res. and Treat., 36, 190-118, 1995, Tanaka et al.,Cancer Res., 58, 3362-3369, 1998). The fact that angiogenesis is prominent in the female reproductive system suggests that treatments against angiogenesis are important for several conditions like bleeding disorders or in the context of birth control (Pepper, Arteriosclerosis, Thrombosis, and Vascular Biology 17:605-619,1997).

Thus one object of the invention is to provide compounds which can be used in the treatment of patients suffering from diseases in which neovascularisation or angiogenesis prevails or for the control of normal angiogenesis to obtain e.g. birth control.

WO 9518793 discloses N-substituted azaheterocyclic carboxylic acids and esters thereof, methods for their preparation, compositions containing them and their use in treatment of hyperalgesic and/or inflammatory conditions.

WO9631497, WO9631498, WO9631499, WO9631481, WO9711071, WO9815548, WO9815546, WO9815550, PCT/DK98/00273, PCT/DK98/00271, DK 0367/98, DK 0366/98, DK 1472/97 and DK 1523/98 discloses N-substituted azaheterocyclic compounds, methods for their preparation, compositions containing them and their use in treatment of hyperalgesic and/or inflammatory conditions as well as as well as their use for treatment of indications caused by or related to the secretion and circulation of insulin antagonising peptides, e.g. non-insulin-dependent diabetes mellitus (NIDDM) and ageing-associated obesity.

### DESCRIPTION OF THE INVENTION

It has surprisingly been found that compounds of the general formulas Ia-Id below can be used in the treatment, prevention, alleviation or amelioration of an indication related to angiogenesis.

Accordingly, the present invention relates to the use of a compound of the following groups of compounds having the general formula Ia

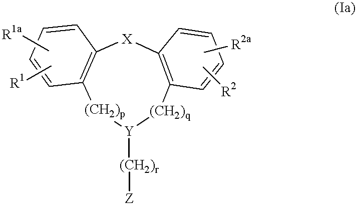

wherein R¹, R^{1a}, R² and R^{2a}, independently are hydrogen, halogen, trifluoromethyl, C₁₋₆-alkyl, C₁₋₆-alkoxy, hydroxy, NR⁷R⁸, cyano, methylthio or —SO₂NR⁷R⁸ wherein R⁷and R⁸ independently are hydrogen or C₁₋₆-alkyl; and

Y is >N—CH₂—, >CH—CH₂— or >C═CH— wherein only the underscored atom participates in the ring system; or

Y is —CH₂N(—)CH₂—, —CH₂N(—)CH₂—, —(C═O)N(—)CH₂—, —CH₂N(—)(C═O)—, —CH₂CH(—)CH₂—, —CH₂CH(—)CH₂—, —CH₂C(—)═CH—, —CH═C(—)CH₂—, —OCH(—)CH₂—, —CH₂CH(—)O—, —SCH(—)CH₂—, —CH₂CH(—)S—, wherein only the underscored atom participates in the ring system; or

Y is >N—, >CH—, >N—(C═O)— or >C═C(R⁸)—, wherein only the underscored atom participates in the ring system and R⁸ is hydrogen or C₁₋₆-alkyl; or

Y is >CH—O— or >CH—S(O)_{y} wherein y is 0, 1 or 2, or —N(R⁸)— wherein R⁸ is hydrogen or C₁₋₆-alkyl, and wherein only the underscored atom participates in the ring system; and

X is completion of an optional bond, ortho-phenylene, —O—, —S—, —C(R⁷R⁸)—, —CH₂CH₂—, —CH═CH—CH₂—, —CH₂—CH═CH—, —CH₂—(C═O)—, —(C═O)—CH₂—, —CH₂CH₂CH₂—, —CH═CH—, —N(R⁸)—(C═O)—, —(C═O)—N(R⁸)—, —O—CH₂—, —CH₂—O—, —OCH₂0—, —CH₂OCH₂—, —S—CH₂—, —CH₂—S—, —(CH₂)N(R⁸)—, —N(R⁸)(CH₂)—, —N(CH₃)SO₂—, —SO₂N(CH₃)—, —CH(R⁹)CH₂—, —CH₂CH(R⁹)—, —(C═O)—, —N(R⁸)— or —(S═O)— wherein R⁷ and R⁸ independently are hydrogen or C₁₋₆-alkyl; and wherein R⁹ is C₁₋₆-alkyl or phenyl; and

p and q independently are 0 or 1; and

r is 0, 1, 2, 3 or 4; and

Z is selected from

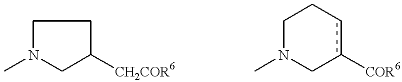

wherein R⁶ is OH or C₁₋₆-alkoxy; and

[symbol] is optionally a single bond or a double bond; or

Z is selected from

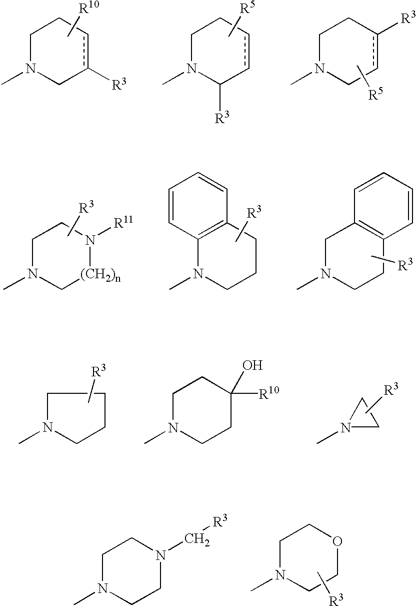

wherein n is 1 or 2;

R³ is —(CH₂)ₘOH or —(CH₂)ₛCOR⁴ wherein m is 0, 1, 2, 3, 4, 5 or 6 and s is 0 or 1 and wherein R⁴ is —OH, —NH₂, —NHOH or C₁₋₆-alkoxy; and

R⁵ is hydrogen, halogen, trifluoromethyl, hydroxy, C₁₋₆-alkyl or C₁₋₆-alkoxy; and

R¹⁰ is hydrogen, C₁₋₆-alkyl, C₁₋₆-alkoxy or phenyl optionally substituted with halogen, trifluoromethyl, hydroxy, C₁₋₆-alkyl or C₁₋₆-alkoxy; and

R¹¹ is hydrogen or C₁₋₆-alkyl; and

[symbol] is optionally a single bond or a double bond; or

Z is selected from

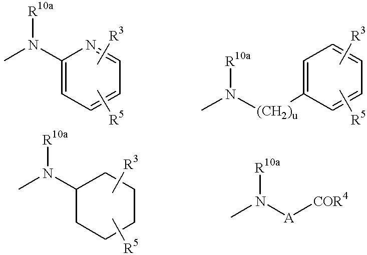

wherein u is 0or 1;

R³ is —(CH₂)ₘOH or —(CH₂)ₛCOR⁴ wherein m is 0, 1, 2, 3, 4, 5 or 6 and s is 0 or 1 and wherein R⁴ is —OH, —NH₂, —NHOH or C₁₋₆-alkoxy; and

R⁵ is hydrogen, halogen, trifluoromethyl, hydroxy, C₁₋₆-alkyl or C₁₋₆-alkoxy; and

R^{10a} is hydrogen or C₁₋₆-alkyl; and

A is C₁₋₆-alkylene, C₂₋₆-alkenylene or C₂₋₆-alkynylene; or

Z is selected from

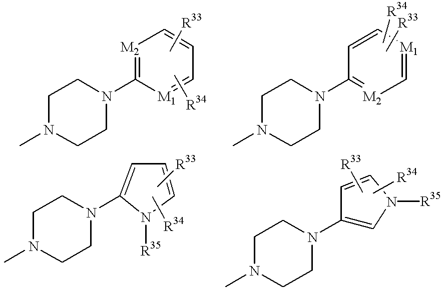

wherein M₁ and M₂ independently are C or N; and

R³⁵ is hydrogen, C₁₋₆-alkyl, phenyl or benzyl; and

R³³ is hydrogen, halogen, trifluoromethyl, nitro or cyano; and

R³⁴ is hydrogen, halogen, trifluoromethyl, nitro, cyano, —(CH₂)_{w}COR³¹, —(CH₂)_{w}OH or —(CH₂)_{w}SO₂R−wherein R³¹ is hydroxy, C₁₋₆-alkoxy or NHR³², wherein R is hydrogen or C₁₋₆-alkyl, and w is 0, 1 or 2; or

R³⁴ is selected from

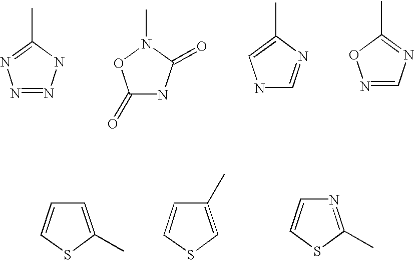

or

Z is

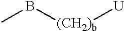

wherein b is 0, 1, 2, 3 or 4; and

B is —CH═CR⁴⁹—, —CR⁴⁹═CH—, —C═C—, —(C═O)—, —(C═CH₂)—, —(CR⁴⁹R⁴⁰)—, —CH(OR⁴¹)—, —CH(NHR⁴¹)—, phenylene, C₃₋₇-cycloalkylene or the completion of a bond, wherein R⁴⁹ and R⁴⁰ independently are hydrogen, C₁₋₆-unbranched alkyl, C₃₋₆-branched alkyl or C₃₋₇-cycloalkyl and wherein R⁴¹ is hydrogen or C₁₋₆alkyl; and

U is

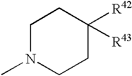

wherein R⁴² is hydrogen, —(CH₂)_{c}OH or —(CH₂)_{d}COR⁴⁷ wherein c is 0, 1, 2, 3, 4, 5 or 6 and d is 0 or 1 and wherein R⁴⁷ is —OH, —NHR⁴⁴ or CI6alkoxy wherein R⁴⁴ is hydrogen or C₁₋₆-alkyl; and

R⁴³ is cyano, —NR⁴⁵R⁴⁶, —NR⁴⁵—V or —(CHR⁴⁸)ₑ—V wherein R⁴⁵ and R⁴⁶ independently are hydrogen or C₁₋₆alkyl and wherein e is 0, 1, 2, 3, 4, 5 or 6 and wherein R⁴⁸ is hydrogen, halogen, cyano, trifluoromethyl, hydroxy, C₁₋₆-alkyl, C₁₋₆-alkoxy, —NR⁴⁵R⁴⁶ or —COOH, and wherein V is C₃₋₈-cycloalkyl, aryl or heteroaryl, which rings may optionally be substituted with one or more halogen, cyano, trifluoromethyl, hydroxy, methylthio, C₁₋₆-alkyl or C₁₋₆-alkoxy; or U is selected from

[chemical structure]

wherein g is 0, 1 or 2; and

R^{11u} is hydrogen, C₁₋₆-alkyl, C₁₋₆-alkoxy or phenyl optionally substituted with halogen, trifluoromethyl, hydroxy, C₁₋₆-alkyl or C₁₋₆-alkoxy; and

R^{12u} is —(CH₂)ₕOH or —(CH₂)ⱼCOR 1⁷u wherein h is 0, 1, 2, 3, 4, 5 or 6 and j is 0 or 1 and wherein R^{17u} is —OH, NHR^{20u} or C₁₋₆-alkoxy wherein R^{20u} is hydrogen or C₁₋₆alkyl; and

R^{13u} is hydrogen, halogen, trifluoromethyl, hydroxy, C₁₋₆-alkyl or C₁₋₆-alkoxy; and

R^{14u} is hydrogen or C₁₋₆-alkyl; and

C is C₁₋₆-alkylene, C₂₋₆-alkenylene or C₂₋₆-alkynylene; and

[symbol] is optionally a single bond or a double bond; and

R^{18u} is selected from

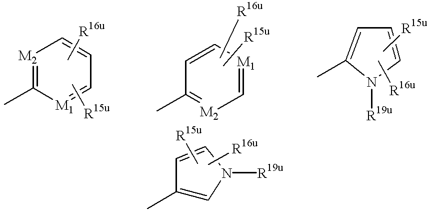

wherein M₁ and M₂ independently are C or N; and

R^{19u} is hydrogen, C₁₋₆-alkyl, phenyl or benzyl; and

R^{15u} is hydrogen, halogen, trifluoromethyl, nitro or cyano; and

R^{16u} is hydrogen, halogen, trifluoromethyl, nitro, cyano, —(CH₂)ₖCOR^{17u}, —(CH₂)ₖOH or —(CH₂)ₖSO₁₇ᵤ wherein k is 0, 1 or 2; or

R^{16u} is selected from

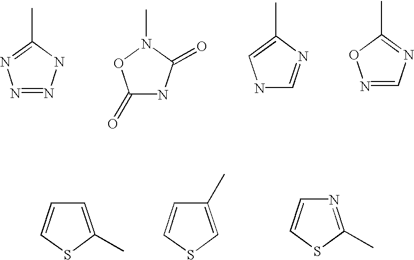

Z is selected from

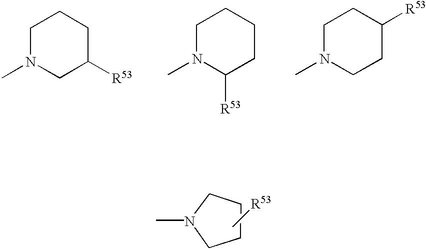

wherein R⁵³ is —(CH₂)ₚₚCOOH wherein pp is 2, 3, 4, 5 or 6; or

Z is

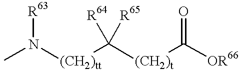

wherein tt and t independently are 0, 1 or 2; and

R⁶³ is H, C₁₋₆-alkyl or optionally substituted benzyl;

R⁶⁴ and R⁶⁵ independently are H, C₁₋₈-alkyl, C₃₋₇-cycloalkyl, phenyl, thienyl, benzyl, or R⁶⁴ and R⁶⁵ together with the C-atom they are attached to form a 3-8 membered carbocyclic ring; and

R⁶⁶ is H or C₁₋₆-alkyl; or

Z is selected from

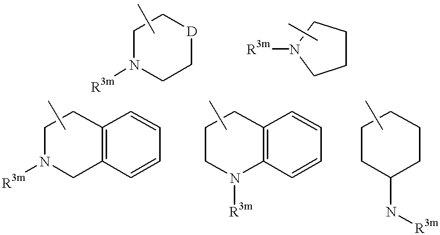

wherein D is —CH₂—, —O—, —S— or —N(R⁷)— wherein R⁷ is hydrogen or C₁₋₆-alkyl; and

R^{3m} is —(CH₂)ₘₘOH or —(CH₂)ₘₚCOR⁴ wherein mm and mp are 1, 2, 3 or 4 and R⁴ is OH, NH₂, NHOH or C₁₋₆alkoxy; or

having the general formula Ib

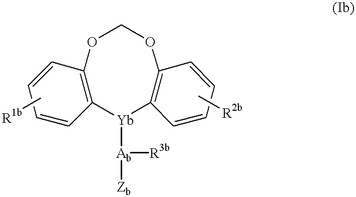

wherein R^{1b} and R^{2b} independently are hydrogen, halogen, trifluoromethyl, hydroxy, C₁₋₆-alkyl or C₁₋₆-alkoxy; and

R^{3b} is hydrogen or C₁₋₃-alkyl; and

A_{b} is C₁₋₃-alkylene; and

Y_{b} is >CH—CH₂—, >C═CH—, >CH—O—, >C═N—, >N—CH₂— wherein only the underscored atom participates in the ring system; and

Z_{b} is selected from

[chemical structure]

wherein nb is 1 or 2; and

R^{11b} is hydrogen or C₁₋₆-alkyl; and

R^{12b} is hydrogen, C₁₋₆-alkyl, C₁₋₆-alkoxy or phenyl optionally substituted with halogen, trifluoromethyl, hydroxy, C₁₋₆-alkyl or C₁₋₆-alkoxy; and

R^{13b} is hydrogen, halogen, trifluoromethyl, hydroxy, C₁₋₆-alkyl or C₁₋₆-alkoxy; and

R^{14b} is —(CH₂)_{mb}OH or —(CH₂)_{tb}COR^{15b} wherein mb is 0, 1, 2, 3, 4, 5 or 6 and tb is 0 or 1 and wherein R^{15b} is —OH, NH₂, —NHOH or C₁₋₆-alkoxy; and

R^{16b} is C₁₋₆-alkyl or —B_{b}—COR^{15b}, wherein B_{b} is C₁₋₆-alkylene, C₂₋₆-alkenylene or C₂₋₆-alkynylene and R^{15b} is the same as above; and

[symbol] is optionally a single bond or a double bond; or

having the general formula Ic

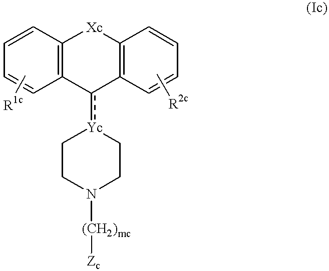

wherein R^{1c} and R^{2c} independently are hydrogen, halogen, trifluoromethyl, hydroxy, C₁₋₆-alkyl or C₁₋₆-alkoxy;

X_{c} is ortho-phenylene, —O—, —S—, —C(R^{6c}R^{7c})—, —CH₂CH₂—, —CH═CH—CH₂—, —CH₂—CH═CH—, —CH₂—(C═O)—, —(C═O)—CH₂—, —CH₂CH₂CH₂—, —CH═CH—, —N(R^{8c})—(C═O)—, —(C═O)—N(R^{8c})—, —O—CH₂—, —CH₂—O—, —OCH₂O—, —S—CH₂—, —CH₂—S—, —(CH₂)N(R^{8c})—, —N(R^{8c})(CH₂)—, —N(CH₃)SO₂—, —SO₂N(CH₃)—, —CH(R^{10c})CH₂—, —CH₂CH(R^{10c})—, —(C═O)—, —N(R^{9c})— or —(S═O)— wherein R^{6c}, R^{7c}, R^{8c} and R^{9c} independently are hydrogen or C₁₋₆-alkyl, and wherein R^{10c} is C₁₋₆-alkyl or phenyl;

Y_{c} is C or N;

[symbol] is optionally a single bond or a double bond, and [symbol] is a single bond when Y_{c} is N;

mc is 1, 2, 3, 4, 5 or 6; and

Z_{c} is —COOR^{3c} or

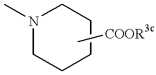

wherein R^{3c} is H or C₁₋₆-alkyl; or

having the general formula Id

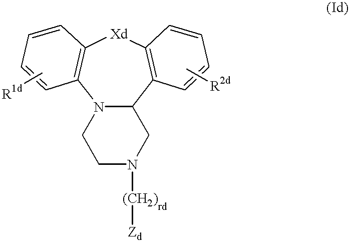

wherein R^{1d} and R^{2d} independently are hydrogen, halogen, trifluoromethyl, hydroxy, C₁₋₆-alkyl or C₁₋₆-alkoxy; and

Xd is —O—, —S— or —S(═O)—; and

rd is 0, 1, 2, 3, 4, 5, 6, 7, 8, 9 or 10; and

Z_{d} is selected from

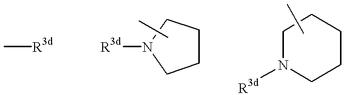

wherein R^{3d} is —(CH₂)_{md}OH or —(CH₂)_{pd}COR^{4d} wherein md and pd independently are 0, 1, 2, 3 or 4 and R^{4d} is OH, NH₂, NHOH or C₁₋₆-alkoxy; or

a pharmaceutically acceptable salt thereof, for the manufacture of a pharmaceutical composition for the treatment, prevention, alleviation or amelioration of a condition related to angiogenesis.

The compounds according to the invention may exist as geometric and optical isomers and all isomers, as separated, pure or partially purified stereoisomers or racemic mixtures thereof are included in the scope of the invention. Isomers may be separated by means of standard methods such as chromatographic techniques or fractional crystallisation of suitable salts.

Preferably, the compounds according to the invention exist as the individual geometric or optical isomers.

The compounds according to the invention may optionally exist as pharmaceutically acceptable acid addition salts, metal salts or, optionally alkylated, ammonium salts.

Examples of such salts include inorganic and organic acid addition salts such as hydrochloride, hydrobromide, sulphate, phosphate, acetate, fumarate, maleate, citrate, lactate, tartrate, oxalate or similar pharmaceutically acceptable inorganic or organic acid addition salts. Further examples of pharmaceutically acceptable inorganic or organic acid addition salts include the pharmaceutically acceptable salts listed in Journal of Pharmaceutical Science, 66, 2 (1977) which are known to the skilled artisan.

Also included are the hydrates of the above mentioned acid addition salts which the present compounds are able to form.

The acid addition salts may be obtained as the direct products of compound synthesis. In the alternative, the free base may be dissolved in a suitable solvent containing the appropriate acid, and the salt isolated by evaporating the solvent or by precipitation or crystallisation.

The compounds according to the invention may be administered in a pharmaceutically acceptable acid addition salt form or where possible as a metal or a lower alkylammonium salt. Such salt forms exhibit approximately the same order of activity as the free base forms.

In the above structural formulas and throughout the present specification, the following terms have the indicated meaning:

The terms “C₁₋₆-alkyl” and “C₁₋₈-alkyl” as used herein, alone or in combination, refers to a straight or branched, saturated hydrocarbon chain having 1 to 6 and 1 to 8 carbon atoms respectively. Examples of such groups include, but are not limited to, methyl, ethyl, n-propyl, iso-propyl, n-butyl, sec-butyl, iso-butyl, tert-butyl, n-pentyl, iso-pentyl, 2-methylbutyl, 3-methylbutyl, n-hexyl, iso-hexyl, 4-methylpentyl, neopentyl, 1,2-dimethylpropyl, 2,2-dimethylpropyl, 1,2,2-trimethylpropyl and the like.

The term “halogen” means fluorine, chlorine, bromine or iodine.

The term “C₁₋₆-alkoxy” as used herein, alone or in combination is intended to include those C₁₋₆-alkyl groups of the designated length in either a linear or branched or cyclic configuration linked thorugh an ether oxygen having its free valence bond from the ether oxygen. Examples of linear alkoxy groups are methoxy, ethoxy, propoxy, butoxy, pentoxy and hexoxy. Examples of branched alkoxy are isoprpoxy, sec-butoxy, tert-butoxy, isopentoxy and isohexoxy. Example of cyclic alkoxy are cyclopropyloxy, cyclobutyloxy, cyclopentyloxy and cyclohexyloxy.

The terms “C₃₋₇-cycloalkyl” and “C₃₋₈-cycloalkyl” as used herein, represents a carbocyclic group having from 3 to 7 carbon atoms and having from 3 to 8 carbon atoms, e.g. cyclopropyl, cyclobutyl, cyclopentyl, cyclohexyl, cycloheptyl and cyclooctyl and the like.

The term “C₃₋₇-cycloalkylene” as used herein represents a bisubstituted carbocyclic group having from 3 to 7 carbon atoms e.g. cyclopropylene, cyclobutylene, cyclopentylene, cyclohexylene and cycloheptylene and the like.

The term “aryl” as used herein is intended to include carbocyclic aromatic ring systems such as phenyl, naphthyl (1-naphthyl or 2-naphthyl), anthracenyl (1-anthracenyl, 2-anthracenyl, 3-anthracenyl), phenanthrenyl, fluorenyl, indenyl and the like.

The term “heteroaryl” as used herein is intended to include heterocyclic aromatic ring systems containing one or more heteroatoms selected from nitrogen, oxygen and sulfur, such as furyl, thienyl, pyrrolyl, oxazolyl, thiazolyl, imidazolyl, isoxazolyl, isothiazolyl, triazolyl, pyranyl, pyridyl, pyridazinyl, pyrimidinyl, pyrazinyl, triazinyl, thiadiazinyl, indolyl, isoindolyl, benzofuryl, benzothienyl, indazolyl, benzimidazolyl, benzthiazolyl, purinyl, quinozolinyl, quinolinyl, isoquinolinyl, quinoxalinyl, naphthyridinyl, pteridinyl, carbazolyl, acridinyl and the like. Heteroaryl is also intended to include the partially or fully hydrogenated derivatives of the heterocyclic systems enumerated above. Non-limiting examples of such partially or fully hydrogenated derivatives are pyrrolinyl, pyrazolinyl, indolinyl, pyrrolidinyl, piperidinyl, piperazinyl, azepinyl, diazepinyl, morpholinyl, thiomorpholinyl, oxazolidinyl, oxazolinyl, oxazepinyl, aziridinyl and tetrahydofuranyl.

The term “3- to 8-membered carbocyclic ring” as used herein refers to a monocyclic unsaturated or saturated ring containing from 3 to 8 carbon atoms. The term includes, but are not limited to cyclopropyl, cyclobutyl, cyclopentyl, cyclohexyl, cycloheptyl and cyclooctyl and the like.

In a preferred embodiment of the invention in formula Ia

R¹, R^{1a}, R² and R^{2a} independently are hydrogen, halogen, trifluoromethyl, C₁₋₆-alkyl or C₁₋₆-alkoxy; and

Y is >N—CH₂—, >CH—CH₂— or >C═CH— wherein only the underscored atom participates in the ring system; and

X is —O—, —S—, —C(R⁷R⁸)—, —CH₂CH₂—, —CH═CH—CH₂—, —CH₂—CH═CH—, —CH₂CH₂CH₂—, —CH═CH—, —N(R⁸)—(C═O)—, —O—CH₂—, —(C═O)— or —(S═O)— wherein R⁷ and R⁸ independently are hydrogen or C₁₋₆-alkyl; and

p and q are 0, and

r is 1, 2 or 3; and

Z is selected from

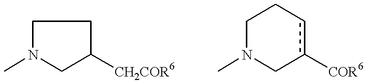

wherein R⁶ is OH or C₁₋₆-alkoxy; and

[symbol] is optionally a single bond or a double bond; or

a pharmaceutically acceptable salt thereof.

Preferred compounds of the present invention include

(R)-1-(3-(10,11-Dihydro-5H-dibenzo[a,d]cyclohepten-5-ylidene)-1-propyl)-3-piperidinecarboxylic acid;

(S)-1-(3-(10,11-Dihydro-5H-dibenzo[a,d]cyclohepten-5-ylidene)-1-propyl)-3-piperidinecarboxylic acid;

1-(3-(10,11-Dihydro-5H-dibenzo[a,d]cyclohepten-5-ylidene)-1-propyl)-1,2,5,6-tetrahydro-3-pyridinecarboxylic acid;

(R)-1-(3-(Fluoren-9-ylidene)-1-propyl)-3-piperidinecarboxylic acid;

1-(3-(5H-Dibenzo[a,d]cyclohepten-5-ylidene)-1-propyl)-3-piperidinecarboxylic acid;

1-(3-(Thioxanthen-9-ylidene)-1-propyl)-3-piperidinecarboxylic acid;

(R)-1-(3-(10,11-Dihydro-5H-dibenz[b,f]azepin-5-yl)-1-propyl)-3-piperidinecarboxylic acid;

(R)-1-(4-(10,11-Dihydro-5H-dibenzo[b,f]azepin-5-yl)-1-butyl)-3-piperidinecarboxylic acid;

(R)-1-(2-(10,11-Dihydro-5H-dibenzo[b,f]azepin-5-yl)ethyl)-3-piperidinecarboxylic acid;

(R)-1-(3-(3-Chloro-10,11-dihydro-5H-dibenzo[b,f]azepin-5-yl)-1-propyl)-3-piperidinecarboxylic acid;

(R)-1-(3-(10H-Phenothiazin-10-yl)-1-propyl)-3-piperidinecarboxylic acid;

(R)-1-(3-(10H-Phenoxazin-10-yl)-1-propyl)-3-piperidinecarboxylic acid;

(S)-1-(3-(10,11-Dihydro-5H-dibenzo[b,f]azepin-5-yl)-1-propyl)-3-piperidinecarboxylic acid;

1-(3-(10,11-Dihydro-5H-dibenzo[b,f]azepin-5-yl)-1-propyl)-3-pyrrolidinacetic acid;

(R)-1-(3-(3-Methyl-10,11-dihydro-5H-dibenzo[a,d]cyclohepten-5-ylidene)-1-propyl)-3-piperidinecarboxylic acid;

(R)-1-(3-(2-Trifluoromethyl-10H-phenothiazin-10-yl)-1-propyl)-3-piperidinecarboxylic acid;

(R)-1-(3-(5-Oxo-10H-phenothiazin-10-yl)-1-propyl)-3-piperidinecarboxylic acid;

(R)-1-(3-(11H-10-Oxa-5-aza-5H-dibenzo[a,d]cyclohepten-5-yl)-1-propyl)-3-piperidinecarboxylic acid;

1-(3-(10,11-Dihydro-5H-dibenzo[b,f]azepin-5-yl)-1-propyl)-1,2,5,6-tetrahydro-3-pyridinecarboxylic acid;

(R)-1-(3-(6,7-Dihydro-5H-dibenzo[b,g]azocin-12-yl)-1-propyl)-3-piperidinecarboxylic acid;

(R)-1-(3-(10,11-Dihydro-5H-dibenzo[a,d]cyclohepten-5-yl)-1-propyl)-3-piperidinecarboxylic acid;

(R)-1-(3-Methoxy-10,11-dihydro-5H-dibenzo[b,f]azepin-5-yl)-1-propyl)-3-piperidinecarboxylic acid;

(R)-1-(3-(10-Methyl-11-oxo-10,11-dihydro-5H-dibenzo[b,e][1 ,4]diazepin-5-yl)-1-propyl)-3-piperidinecarboxylic acid;

(R)-1-(3-(9(H)-Oxo-10H-acridin-10-yl)-1-propyl)-3-piperidinecarboxylic acid;

(R)-1-(2-(10,11-Dihydro-5H-dibenzo[a,d]cyclohepten-5-ylidene)-1-ethyl)-3-piperidinecarboxylic acid hydrochloride;

(R)-1-(2-(6,11-Dihydrodibenz[b,e]oxepin-I I -ylidene)-1-ethyl)-3-piperidinecarboxylic acid hydrochloride;

(R)-1-(3-(2-Chloro-10,11-dihydro-5H-dibenzo[a,d]cyclohepten-5-ylidene)-1-propyl)-3-piperidinecarboxylic acid hydrochloride;

(R)-1-(3-(2-Bromo-10,11-dihydro-5H-dibenzo[a,d]cyclohepten-5-ylidene)-1-propyl)-3-piperidinecarboxylic acid hydrochloride;

(R)-1-(3-(2-Fluoro-10,11-dihydro-5H-dibenzo[a,d]cyclohepten-5-ylidene)-1-propyl)-3-piperidinecarboxylic acid hydrochloride;

(R)-1-(3-(2-lodo-10,11-dihydro-5H-dibenzo[a,d]cyclohepten-5-ylidene)-1-propyl)-3-piperidinecarboxylic acid hydrochloride;

(Z)-(R)-1-(3-(2-lodo-10,11-dihydro-5H-dibenzo[a,d]cyclohepten-5-ylidene)-1-propyl)-3-piperidinecarboxylic acid hydrochloride;

(E)-(R)-1-(3-(2-lodo-10,11-dihydro-5H-dibenzo[a,d]cyclohepten-5-ylidene)-1-propyl)-3-piperidinecarboxylic acid hydrochloride;

(R)-1-(3-(2-Methoxy-10,11-dihydro-5H-dibenzo[a,d]cyclohepten-5-ylidene)-1-propyl)-3-piperidinecarboxylic acid hydrochloride.

In another preferred embodiment of the invention in formula Ia

R¹, R^{1a}, R² and R^{2a} independently are hydrogen, halogen, trifluoromethyl, hydroxy, C₁₋₆-alkyl or C₁₋₆-alkoxy; and

Y is —CH₂N(—)CH₂—, —CH₂N(—)CH₂—, —(C═O)N(—)CH₂—, —CH₂N(—)(C═O)—, —CH CH(—)CH₂—, —CH₂CH(—)CH₂—, —CH₂C(—)═CH—, —CH═C(—)CH₂—, —OCH(—)CH₂—, —CH₂CH(—)O—, —SCH(—)CH₂—, —CH₂CH(—)S—, wherein only the underscored atom participates in the ring system; and

X is —O—, —S—, —C(R⁷R⁸)—, —CH₂CH₂—, —CH═CH—CH₂—, —CH₂—CH═CH—, —CH₂—(C═O)—, —(C═O)—CH₂—, —CH₂CH₂CH₂—, —CH═CH—, —N(R⁸)—(C═O)—, —(C═O)—N(R⁸)—, —O—CH₂—, —CH₂—O—, —S—CH₂—, —CH₂—S—, —N(R⁸)—, —(C═O)— or —(S═O)— wherein R⁷ and R⁸ independently are hydrogen or C₁₋₆-alkyl; and

p and q independently are 0 or 1; and

r is 1, 2 or 3; and

Z is selected from

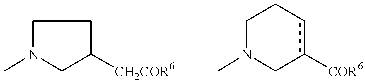

wherein R⁶ is OH or C₁₋₆-alkoxy; and

[symbol] is optionally a single bond or a double bond; or

a pharmaceutically acceptable salt thereof.

Further preferred compounds of the invention include:

(R)-1-(3-(6,11-Dioxo-6,11-dihydro-5H-dibenz[b,e]azepin-5-yl)-1-propyl)-3-piperidinecarboxylic acid;

(R)-1-(3-(6,11-Dihydro-5H-dibenz[b,e]azepin-5-yl)-1-propyl)-3-piperidinecarboxylic acid;

(R)-1-(3-(5,11-Dihydro-10H-dibenzo[b,e][1 ,4]diazepin-10-yl)-1-propyl)-3-piperidinecarboxylic acid;

(R)-1-(3-(11H-Dibenzo[b,f][1,4]thiazepin-10-yl)-1-propyl)-3-piperidinecarboxylic acid;

(R)-1-(3-(11H-Dibenz[b,f][1,4]oxazepin-10-yl)-1-propyl)-3-piperidinecarboxylic acid;

(R)-1-(3-(11H-Dibenz[b,f][1,4]oxathiepin-11-yl)-1-propyl)-3-piperidinecarboxylic acid;

(R)-1-(3-(11H-Dibenzo[b,e][1,4]dithiepin-11-yl)-1-propyl)-3-piperidinecarboxylic acid;

(R)-1-(3-(11H-Dibenz[b,e][1,4]oxathiepin-10-yl)-1-propyl)-3-piperidinecarboxylic acid;

(R)-1-(3-(11,12-Dihydro-10H-dibenz[b,g][1,5]oxazocin-11yl)-1-propyl)-3-piperidinecarboxyic acid;

(R)-1-(3-(11,12-Dihydro-10H-dibenzo[b,g][1,5]thiazocin-11-yl)-1-propyl)-3-piperidinecarboxylic acid;

1-(3-(11,12-Dihydro-6H-dibenz[b,f]azocin-5-yl)-1-propyl)-3-piperidinecarboxylic acid;

1-(3-(11,12-Dihydro-5H-dibenzo[a,e]cycloocten-5-ylidene)-1-propyl)-3-piperidinecarboxylic acid;

1-(3-(6-Oxo-11,12-dihydro-5H-dibenz[b,f]azocin-5 -yl)-1-propyl)-3- piperidinecarboxylic acid;

1-(3-(7,12-Dihydro-6H-dibenzo[a,d]cycloocten-6-ylidene)-1-propyl)-3-piperidinecarboxylic acid;

1-(3-(5-Methyl-5,11-dihydro-dibenz[b,f]azepin-10-ylidene)-1-propyl)-3-piperidinecarboxylic acid;

1-(3-(6-Oxo-5,11-dihydro-5H-dibenz[b,e]azepin-5-yl)-1-propyl)-3-piperidinecarboxylic acid;

(R)-1-(3-(11-Oxo-10,11-dihydro-5H-dibenzo[b,e][1,4]diazepin-10-yl)-1-propyl)-3-piperidinecarboxylic acid;

(R)-1-(3-(6-Oxo-11,12-dihydro-5H-dibenz[b,f]azocin-5-yl)-1-propyl)-3-piperidinecarboxylic acid;

(R)-1-(3-(10,11-Dihydro-dibenz[b,f][1,4]oxazepin-10-yl)-1-propyl)-3-piperidinecarboxylic acid;

(R)-1-(3-(5,6,11,12-Tetrahydro-dibenz[b,f]azocin-5-yl)-1-propyl)-3-piperidinecarboxylic acid;

(R)-1-(3-(11-Oxo-6,11-dihydro-5H-dibenz[b,e]azepin-5-yl)-1-propyl)-3-piperidinecarboxylic acid;

(R)-1-(3-(5-Methyl-dibenz[b,f]azepin-10-yl)-1-propyl)-3-piperidinecarboxylic acid;

(R)-1-(3-(6,7-Dihydro-5H-dibenz[b,g][1,5]oxazocin-6-yl)-1-propyl)-3-piperidinecarboxylic acid;

(R)-1-(3-(11,12-Dihydro-dibenz[a ,e]cycloocten-5-yl)-1-propyl)-3-piperid inecarboxylic acid.

In another preferred embodiment of the invention in formula Ia

R¹, R^{1a}, R² and R^{2a} independently are hydrogen, halogen, trifluoromethyl, NR⁷R⁸, hydroxy, C₁₋₆-alkyl or C₁₋₆-alkoxy wherein R⁷ and R⁸ independently are hydrogen or C₁₋₆-alkyl; and

Y is >N—CH₂—, >CH—CH₂— or >C═CH— wherein only the underscored atom participates in the ring system; and

X is —O—, —S—, —C(R⁷R⁸)—, —CH₂CH₂—, —CH═CH—CH₂—, —CH₂—CH═CH—, —CH₂—(C═O)—, —(C═O)—CH₂—, —CH₂CH₂CH₂—, —CH═CH—, —N(R⁸)—(C═O)—, —(C═O)—N(R⁸)—, —O—CH₂—, —CH₂—O—, —S—CH₂—, —CH₂—S—, —N(R⁸)—, —(C═O)— or —(S═O)— wherein R⁷ and R⁸ independently are hydrogen or C₁₋₆-alkyl; and

p and q are 0; and

r is 1, 2or 3; and

Z is selected from

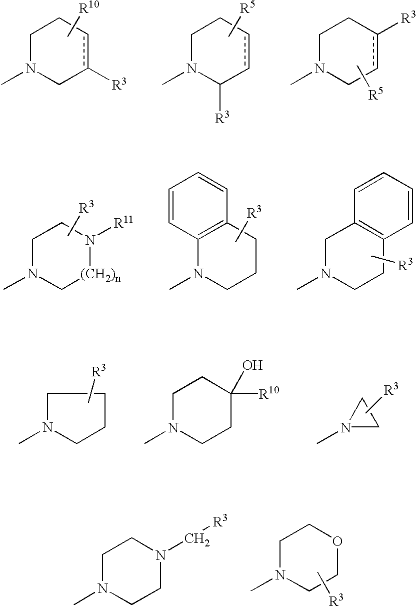

wherein n is 1 or 2; and

R³ is —(CH₂)ₘOH or —(CH₂)ₛCOR⁴ wherein m is 0, 1, 2, 3, 4, 5 or 6 and s is 0 or 1 and wherein

R⁴ is —OH, —NH₂, —NHOH or C₁₋₆-alkoxy; and

R⁵ is hydrogen, halogen, trifluoromethyl, hydroxy, C₁₋₆-alkyl or C₁₋₆-alkoxy; and

R¹⁰ is hydrogen, C₁₋₆-alkyl, C₁₋₆-alkoxy or phenyl optionally substituted with halogen, trifluoromethyl, hydroxy, C₁₋₆-alkyl or C₁₋₆-alkoxy; and

R¹¹ is hydrogen or C₁₋₆-alkyl; and

[symbol] is optionally a single bond or a double bond; or

a pharmaceutically acceptable salt thereof.

Further preferred compounds of the invention include:

1-(3-(10,11-Dihydro-5H-dibenz[b,f]azepin-5-yl)-1-propyl)-3-piperidine-carboxamide;

1-(3-(10,11-Dihydro-5H-dibenz[b,f]azepin-5-yl)-1-propyl)-4-piperidinecarboxylic acid;

1-(3-(10,11-Dihydro-5H-dibenz[b,f]azepin-5-yl)-1-propyl)-2-piperidinecarboxylic acid;

(1-(3-(10,11-Dihydro-5H-dibenz[b,f]azepin-5-yl)-1-propyl)-3-piperidinyl)methanol;

4-(4-Chlorophenyl)-1-(3-(10,11-dihydro-5H-dibenz[b,f]azepin-5-yl)-1-propyl)-4-piperidinol;

4-(3-(10,11-Dihydro-5H-dibenz[b,f]azepin-5-yl)-1-propyl)-2-piperazinecarboxylic acid;

(2S,4R)-1-(3-(10,11-Dihydro-5H-dibenz[b,f]azepin-5-yl)-1-propyl)-4-hydroxy-2-pyrrolidinecarboxylic acid;

4-(3-(10,11-Dihydro-5H-dibenz[b,f]azepin-5-yl)-1-propyl)-2-morpholinecarboxylic acid;

1-(3-(10,11-Dihydro-5H-dibenz[b,f]azepin-5-yl)-1-propyl)-2-aziridinecarboxylic acid;

2-(3-(10,11-Dihydro-5H-dibenz[b,f]azepin-5-yl)-1-propyl)-1,2,3,4-tetrahydro-4-isoquinolinecarboxylic acid;

1-(3-(10,11-Dihydro-5H-dibenz[b,f]azepin-5-yl)-1-propyl)-4-methyl-[1,4]-diazepane-6-carboxylic acid;

2-(3-(10,11-Dihydro-5H-dibenz[b,f]azepin-5-yl)-1-propyl)-1,2,3,4-tetrahydro-3-isoquinolinecarboxylic acid;

1-(3-(10,11-Dihydro-5H-dibenz[b,f]azepin-5-yl)-1-propyl)-3-piperidinecarboxylic acid hydroxamide;

(4-(3-(10,11-Dihydro-5H-dibenz[b,f]azepin-5-yl)-1-propyl)piperazin-1-yl)acetic acid;

1-(3-(10,11-Dihydro-5H-dibenz[b,f]azepin-5-yl)-1-propyl)-4-piperidinecarboxylic acid;

4-(3-(10,11-Dihydro-5H-dibenz[b,f]azepin-5-yl)-1-propyl)-2-piperazinecarboxylic acid;

1-(3-(10,11-Dihydro-5H-dibenz[b,f]azepin-5-yl)-1-propyl)-4-piperidineacetic acid;

1-(3-(10,11-Dihydro-5H-dibenzo[a,d]cyclohepten-5-ylidene)-1-propyl)-4-piperidinecarboxylic acid;

(R)-1-(3-(10,11-Dihydro-5H-dibenzo[a,d]cyclohepten-5-ylidene)-1-propyl)-3-piperidinecarboxamide;

(R)-1-(3-(10,11-Dihydro-5H-dibenzo[a,d]cyclohepten-5-ylidene)-1-propyl)-2-pyrrolidinecarboxylic acid;

(S)-1-(3-(10,11-Dihydro-5H-dibenzo[a,d]cyclohepten-5-ylidene)-1-propyl)-2-pyrrolidinecarboxylic acid;

1-(3-(10,11-Dihydro-5H-dibenzo[a,d]cyclohepten-5-ylidene)-1-propyl)-2-piperidinecarboxylic acid;

1-(3-(10H-Phenoxazin-10-yl)-1-propyl)-4-piperidinecarboxylic acid;

1-(3-(3-Chloro-10,11-dihydro-5H-dibenz[b,f]azepin-5-yl)-1-propyl)-4-piperidinecarboxylic acid;

1-(3-(10,11-Dihydro-5H-dibenz[b,f]azepin-5-yl)-1-propyl)-3-piperidineacetic acid;

1-(3-(10,11-Dihydro-5H-dibenz[b,f]azepin-5-yl)-1-propyl)-2-methyl-3-piperidinecarboxylic acid;

1-(3-(10,11-Dihydro-5H-dibenz[b,f]azepin-5-yl)-1-propyl)-3-q u inuclidiniumcarboxylate;

1-(3-(2,8-Dibromo-10,11-dihydro-5H-dibenz[b,f]azepin-5-yl)-1-propyl)-4-piperidinecarboxylic acid;

1-(3-(3,7-Dichloro-10,11-dihydro-5H-dibenz[b,f]azepin-5-yl)-1-propyl)-4-piperidinecarboxylic acid;

1-(3-(3-Methyl-10,11-dihydro-5H-dibenzo[a,d]cyclohepten-5-ylidene)-1-propyl-4-piperidinecarboxylic acid;

1-(3-(3,7-Dimethyl-10,11-dihydro-5H-dibenz[b,f]azepin-5-yl)-1-propyl)-4-piperidinecarboxylic acid;

1-(3-(3-Dimethylamino-10,11-dihydro-5H-dibenz[b,f]azepin-5-yl)-1-propyl)-4-piperidine-carboxylic acid;

(R)-1-(3-(10,11-Dihydro-5H-dibenzo[a,d]cyclohepten-5-ylidene)-1-propyl)-2-piperidinecarboxylic acid;

(S)-1-(3-(10,11-Dihydro-5H-dibenzo[a,dicyclohepten-5-ylidene)-1-propyl)-2-piperidinecarboxylic acid;

1-(2-(6,11-Dihydrodibenzo[b,e]thiepin-11-ylidene)-1-ethyl)-3-piperidinecarboxylic acid;

1-(2-(6,11-Dihydrodibenzo[b,e]thiepin-11-ylidene)-1-ethyl)-4-piperidinecarboxylic acid;

1-(2-(2-Cloro-6,11-dihydrodibenzo[b,e]thiepin-1-ylidene)-1-ethyl)-3-piperidinecarboxylic acid;

1-(2-(2-Chloro-6,11-dihydrodibenzo[b,e]thiepin-11-ylidene)-1-ethyl)-4-piperidinecarboxylic acid;

(R)-1-(2-(6,11-Dihydrodibenzo[b,e]thiepin-11-ylidene)-1-ethyl)-3-piperidinecarboxyic acid;

1-(3-(2-Bromo-10,11-dihydro5H-dibenzo[a,dlcyclohepten-5-ylidene)-1-propyl)-3-pyrrolidineacetic acid;

1-(3-(3-Methyl-10,11-dihydro-5H-dibenzo[a,d]cyclohepten-5-ylidene)-1-propyl)-3-pyrrolidineacetic acid;

1-(3-(6,11-Dihydro-dibenz[b,e]thiepin-11-ylidene)-1-propyl)-4-piperidinecarboxylic acid;

1-(3-(2-Fluoro-10,11-dihydro-5H-dibenzo[a,d]cyclohepten-5-ylidene)-1-propyl)-4-piperidinecarboxylic acid;

1-(3-(10,11-Dihydro-5H-dibenzo[b,f]azepin-5-yl)-1-propyl)-2-piperidineacetic acid;

1-(3-(Phenothiazin-10-yl)-1-propyl)-4-piperidinecarboxylic acid;

(R)-1-(2-(10,11-dihydro-5H-dibenzo[a,d]cyclohepten-5-ylidene)-1-ethyl)-2-piperidinecarboxylic acid;

1-(2-(10,11-Dihydro-5H-dibenzo[a,d]cyclohepten-5-ylidene)-1-ethyl)-4-piperidinecarboxylic acid;

1-(2-(6,11-Dihydrodibenzo[b,e]oxepin-11-ylidene)-1-ethyl)-4-piperidinecarboxylic acid.

In another preferred embodiment of the invention in formula Ia

R¹, R^{1a}, R² and R^{2a} independently are hydrogen, halogen, trifluoromethyl, hydroxy, C₁₋₆-alkyl or C₁₋₆-alkoxy; and

Y is >N—CH₂—, >CH—CH₂— or >C═CH— wherein only the underscored atom participates in the ring system; and

X is ortho-phenylene, —CH₂—(C═O)—, —(C═O)—CH₂—, —S—CH₂—, —CH₂—S—, —(CH₂)N(R⁸)—, —N(R⁸)(CH₂)—, —N(CH₃)SO₂—, —SO₂N(CH₃)—, —CH(R⁹)CH₂— or —CH₂CH(R⁹)— wherein R⁸ is hydrogen or C₁₋₆-alkyl and R⁹ is C₁₋₆-alkyl or phenyl; and

p and q are 0; and

r is 1, 2 or 3; and

Z is selected from

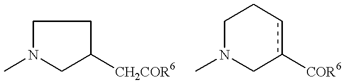

wherein R⁶ is OH or C₁₋₆-alkoxy; and

[symbol] is optionally a single bond or a double bond; or

a pharmaceutically acceptable salt thereof.

Further preferred compounds of the invention include:

1-(3-(9H-Tribenz[b,d,f]azepin-9-yl)-1-propyl)-3-piperidinecarboxylic acid;

1-(3-(Tribenzo[a,c,e]cyclohepten-9-ylidene)-1-propyl)-3-piperidinecarboxylic acid;

1-(3-(5-Methyl-5,6-dihydrodibenz[b,e]azepin-11-ylidene)-1-propyl)-3-piperidinecarboxylic acid;

1-(3-(6-Methyl-6H-dibenzo[c,f][1,2]thiazepin-5,5-dioxide-11-ylidene)-1-propyl)-3-piperidinecarboxylic acid;

1-(3-(10-Methyl-10,11-dihydro-5H-dibenzo[b,e]cyclohepten-5-ylidene)-1-propyl)-3-piperidinecarboxylic acid;

1-(3-(10-Phenyl-10,11-dihydro-5H-dibenzo[b,e]cyclohepten-5-ylidene)-1-propyl)-3-piperidinecarboxylic acid;

1-(3-(6,11-Dihydro-11H-dibenzo[b,e][1,4]thiazepin-11-yl)-1 propyl)-3-piperidinecarboxylic acid;

1-(3-(10-Methyl-10,11-dihydro-dibenzo[b,e][1,4]diazepin-5-yl)-1-propyl)-3-piperidinecarboxylic acid;

(R)-1-(3-(10-Oxo-10,11-dihydro-5H-dibenz[b,f]azepin-5-yl)-1-propyl)-3-piperidinecarboxylic acid;

(R)-1-(3-(6-Methyl-6,11-dihydro-dibenzo[c,f][1,2,5]thiadiazepin-5,5-dioxide-11-yl)-1-propyl)-3-piperidinecarboxylic acid;

(R)-1-(3-(5-Methyl-5,6-dihydrodibenz[b,e]azepin-11-ylidene)-1-propyl)-3-piperidinecarboxylic acid;

(R)-1-(3-(9H-Tribenzo[a,c,e]cyclohepten-9-ylidene)-1-propyl)-3-piperidinecarboxylic acid;

(R)-1-(3-(9H-Tribenzo[b,d,f]azepine-9-yl)propyl)-3-piperidinecarboxylic acid.

In another preferred embodiment of the invention in formula Ia

R¹, R^{1a}, R² and R^{2a} independently are hydrogen, halogen, trifluoromethyl, hydroxy, C₁₋₆-alkyl or C₁₋₆-alkoxy; and

Y is >N—CH₂—, >CH—CH₂— or >C═CH— wherein only the underscored atom participates in the ring system; and

X is —O—, —S—, —C(R⁷R⁸), —CH₂CH₂—, —CH═CH—CH₂—, —CH₂—CH═CH—, —CH₂—(C═O)—, —(C═O)—CH₂—, —CH₂CH₂CH₂—, —CH═CH—, —N(R⁸)—(C═O)—, —(C═O)—N(R⁸)—, —O—CH₂—, —CH₂—O—, —S—CH₂—, —CH₂—S—, —N(R⁸)—, —(C═O)— or —(S═O)— wherein R⁷ and R⁸ independently are hydrogen or C₁₋₆-alkyl; and

p and q are 0; and

r is 1, 2 or 3; and

Z is selected from

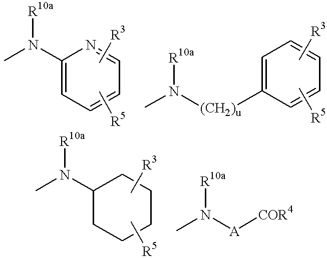

wherein u is 0 or 1;

R³ is —(CH₂)ₘOH or —(CH₂)ₛCOR⁴ wherein m is 0, 1, 2, 3, 4, 5 or 6 and s is 0 or 1 and wherein

R⁴is —OH, —NH₂, —NHOH or C₁₋₆-alkoxy; and

R⁵ is hydrogen, halogen, trifluoromethyl, hydroxy, C₁₋₆-alkyl or C₁₋₆-alkoxy; and

R^{10a} is hydrogen or C₁;₆-alkyl; and

A is C₁₋₆-alkylene, C₂₋₆-alkenylene or C₂₋₆-alkynylene; or

a pharmaceutically acceptable salt thereof.

Further preferred compounds of the invention include:

3-(N-Methyl-N-(3-(10,11-dihydrodibenzo[a,d]cyclohepten-5-ylidene)-1-propyl)amino)propionic acid;

4-(N-Methyl-N-(3-(10,11-dihydrodibenzo[a,d]cyclohepten-5-ylidene)-1-propyl)amino)butyric acid;

3-((3-(10,11-Dihydro-5H-dibenz[b,f]azepin-5-yl)-1-propyl)amino)propionic acid;

2-(N(3-(10,11-Dihydro-5H-dibenz[b,f]azepin-5-yl)-1-propyl)-N-methyl-amino)succinic acid;

2-((3-(10,11-Dihydro-5H-dibenz[b,f]azepin-5-yl)-1-propyl)amino)benzoic acid;

2-(N-(3-(10,11-Dihydro-5H-dibenz[b,f]azepin-5-yl)-1-propyl)-N-methylamino)nicotinic acid;

2-((N-(3-(10,11-Dihydro-5H-dibenz[b,f]azepin-5-yl)-1-propyl)-N-methylamino)methyl)benzoic acid;

2-((N-(3-(10,11-Dihydro-5H-dibenz[b,f]azepin-5-yl)-1-propyl)-N-methylamino)-1-cyclohexanecarboxylic acid;

2-(3-(10,11-Dihydro-5H-dibenz[b,f]azepin-5-yl)-1-propylamino)pyridin-3-ol;

3-((3-(10,11-Dihydro-5H-dibenz[b,f]azepin-5-yl)-1-propyl)amino)benzoic acid;

2-((3-(10,11-Dihydro-5H-dibenzo[a,d]cyclohepten-5-ylidene)-1-propyl)amino)benzoic acid;

2-(N-(3-(3-Chloro-10,11-dihydro-5H-dibenz[b,f]azepin-5-yl)-1-propyl)amino)benzoic acid;

5-Bromo-2-(N-(3-(10,11-dihydro-5H-dibenzo[a,d]cyclohepten-5-ylidene)-1-propyl)amino)benzoic acid.

In another preferred embodiment of the invention in formula Ia

R¹, R^{1a}, R² and R^{2a} independently are hydrogen, halogen, trifluoromethyl, hydroxy, C₁₋₆-alkyl or C₁₋₆-alkoxy;

Y is >N—CH₂—, >CH—CH₂—, >C═CH— or >CH—O— wherein only the underscored atom participates in the ring system; and

X is ortho-phenylene, —O—, —S—, —C(R⁷R⁸)—, —CH₂CH₂—, —CH═CH—CH₂—, —CH₂—CH═CH—, —CH₂—(C═O)—, —(C═O)—CH₂—, —CH₂CH₂CH₂—, —CH═CH—, —N(R⁸)—(C═O)—, —(C═O)—N(R⁸)—, —O—CH₂—, —CH₂—O—, —OCH₂O—, —S—CH₂—, —CH₂—S—, —(CH₂)N(R⁸)—, —N(R⁸)(CH₂)—, —N(CH₃)SO₂—, —SO₂N(CH₃)—, —CH(R⁹)CH₂—, —CH₂CH(R⁹)—, —(C═O)—, —N(R⁸)— or —(S═O)— wherein R⁷ and R⁸ independently are hydrogen or C₁₋₆-alkyl; and wherein R⁹ is C₁₋₆-alkyl or phenyl; and

p and q are 0; and

r is 1, 2 or 3; and

Z is selected from

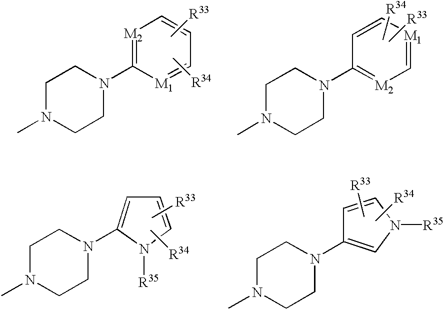

wherein M₁ and M₂ independently are C or N; and

R³⁵ is hydrogen, C₁₋₆-alkyl, phenyl or benzyl; and

R³³ is hydrogen, halogen, trifluoromethyl, nitro or cyano; and

R³⁴ is hydrogen, halogen, trifluoromethyl, nitro, cyano, —(CH₂)_{w}COR³¹, —(CH₂)_{w}OH or —(CH₂)_{w}SO₂R³¹ wherein R³¹ is hydroxy, C₁₋₆-alkoxy or NHR³², wherein R³² is hydrogen or C₁₋₆-alkyl, and w is 0, 1 or 2; or

R³⁴ is selected from

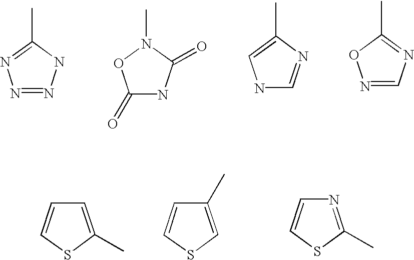

or a pharmaceutically acceptable salt thereof.

Further preferred compounds of the invention include:

2-(4-(3-(12H-Dibenzo[d,g][1,3]dioxocin-12-ylidene)-1-propyl)piperazin-1-yl)-3-pyridinecarboxylic acid;

2-(4-(3-(2,10-Dichloro-12H-dibenzo[d,g][1 ,3]dioxocin-12-ylidene)-1-propyl)-piperazin-1-yl)-3-pyridinecarboxylic acid;

2-(4-(3-(12H-Dibenzo[d,g][1,3,6]dioxazocin-12-yl)-1-propyl)piperazin-1-yl)-3-pyridinecarboxylic acid;

2-(4-(3-(2-Chloro-12H-dibenzo[d,g][1 ,3,6]dioxazocin-12-yl)-1-propyl)-piperazin-1-yl)-3-pyridinecarboxylic acid;

1-(3-(10,11-Dihydro-5H-dibenzo[a,d]cyclohepten-5-ylidene)-1-propyl)-4-(2-pyridyl)piperazine;

2-(4-(3-(10,11-Dihydro-5H-dibenzo[a,d]cyclohepten-5-ylidene)-propyl)-1-piperazinyl)-3-pyridine-carboxylic acid; 2-(4-(2-(10,11-Dihydro-5H-dibenz[b,f]azepin-5-yl)-1-ethyl)-1-piperazinyl)-3-pyridinecarboxylic acid;

6-(4-(3-(10,11-Dihydro-5H-dibenz[b,f]azepin-5-yl)-1-propyl)-1-piperazinyl)-2-pyridinecarboxylic acid;

2-(4-(3-(10,11-Dihydro-5H-dibenz[b,f]azepin-5-yl)-1-propyl)-1-piperazinyl)-3-pyridinecarboxylic acid;

2-(4-(3-(10,11-Dihydro-5H-dibenzo[b,f]azepin-5-yl)-1-propyl)-1-piperazinyl)-5-pyridinecarboxylic acid;

2-(4-(3-(3-Chloro-10,11-dihydro-5H-dibenzo[b,f]azepin-5-yl)-1-propyl)-1-piperazinyl)3-pyridinecarboxylic acid;

1-(3-(10,11-Dihydro-5H-dibenzo[a,d]cyclohepten-5-ylidene)-1-propyl)-4-(2-nitrophenyl)-piperazine;

2-(4-(3-(10,11-Dihydro-5H-dibenzo[a,d]cyclohepten-5-ylidene)-1-propyl)-1-piperazinyl)-benzonitrile;

2-(4-(3-(10,11-Dihydro-5H-dibenzo[a,d]cyclohepten-5-ylidene)-1-propyl)-1-piperazinyl)-benzoic acid;

1-(3-(10,11-Dihydro-5H-dibenzo[a,d]cyclohepten-5-ylidene)-1-propyl)-4-(3-trifluoromethyl-2-pyridyl)piperazine;

2-(4-(2-(6,11-Dihydro-dibenzo[b,e]thiepin-11-ylidene)ethyl)piperazin-1-yl)-3-pyridinecarboxylic acid;

2-(4-(3-(6,11-Dihydrodibenzo[b,e]thiepin-11-ylidene)-1-propyl)-1-piperazinyl)-3-pyridinecarboxylic acid;

2-(4-(2-(6,11-Dihydrodibenzo[b,e]thiepin-11-yloxy)ethyl)-1-piperazinyl)-3-pyridinecarboxylic acid;

6-(4-(3-(10,11-Dihydro-5H-dibenzo[a,d]cyclohepten-5-ylidene)-1-propyl)piperazin-1-yl)-2-pyridinecarboxylic acid;

2-(4-(3-(3-Methyl-10,11-dihydro-5H-dibenzo[b,f]azepin-5-yl)-1-propyl)-1-piperazinyl)-3-pyridinecarboxylic acid;

6-(4-(3-(Dibenzo[d,g][1,3,6]dioxazocin-12-yl)-1-propyl)-piperazin-1-yl)-pyridine-2-carboxylic acid.

In another preferred embodiment of the invention in formula Ia

R¹, R^{1a}, R² and R^{2a} independently are hydrogen, halogen, trifluoromethyl, hydroxy, C₁₋₆-alkyl or C₁₋₆-alkoxy; and

Y is >N—, >CH—, >N—(C═O)— or >C═C(R⁸)—, wherein only the underscored atom participates in the ring system and R⁸ is hydrogen or C₁₋₆-alkyl; and

X is ortho-phenylene, —O—, —S—, —C(R⁷R⁸)—, —CH₂CH₂—, —CH═CH—CH₂—, —CH₂—CH═CH—, —CH₂—(C═O)—, —(C═O)—CH₂—, —CH₂CH₂CH₂—, —CH═CH—, —N(R⁸)—(C═O)—, —(C═o)—N(R⁸)—, —O—CH₂—, —CH₂—O—, —OCH₂O—, —CH₂OCH₂—, —S—CH₂—, —CH₂—S—, —(CH₂)N(R⁸)—, —N(R⁸)(CH₂)—, —N(CH₃)SO₂—, —SO₂N(CH₃)—, —CH(R⁹)CH₂—, —CH₂CH(R⁹)—, —(C═O)—, —N(R⁸)— or —(S═O)— wherein R⁷ and R⁸ independently are hydrogen or C₁₋₆-alkyl; and wherein R⁹ is C₁₋₆-alkyl or phenyl;

and p and q are 0; and

r is 0, 1, 2, 3 or 4; and

Z is

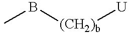

wherein b is 0, 1, 2, 3 or 4; and p1 B is —CH═CR⁴⁹—, —CR⁴⁹═CH—, —C═C—, —(C═O)—, —(C═CH₂)—, —(CR⁴⁹R⁴⁰)—, —CH(OR⁴¹)—, —CH(NHR⁴¹)—, phenylene, C₃₋₇-cycloalkylene or the completion of a bond, wherein R⁴⁹ and R⁴⁰ independently are hydrogen, C₁₋₆-unbranched alkyl, C₃₋₆-branched alkyl or C₃₋₇-cycloalkyl and wherein R⁴¹ is hydrogen or C₁₋₆-alkyl; and

U is selected from

[chemical structure]

wherein g is 0, 1 or 2; and

R^{11u} is hydrogen, C₁₋₆-alkyl, C₁₋₆-alkoxy or phenyl optionally substituted with halogen, trifluoromethyl, hydroxy, C₁₋₆-alkyl or C₁₋₆-alkoxy; and

R^{12u} is —(CH₂)ₕOH or —(CH₂)ⱼCOR^{17u} wherein h is 0, 1, 2, 3, 4, 5 or 6 and j is 0 or 1 and wherein R^{17u} is —OH, —NHR^{20u} or C₁₋₆-alkoxy wherein R^{20u} is hydrogen or C₁₋₆-alkyl; and

R^{13u} is hydrogen, halogen, trifluoromethyl, hydroxy, C₁₋₆-alkyl or C₁₋₆-alkoxy; and

R^{14u} is hydrogen or C₁₋₆-alkyl; and

C is C₁₋₆-alkylene, C₂₋₆-alkenylene or C₂₋₆-alkynylene; and

[symbol] is optionally a single bond or a double bond; and

R^{18u} is selected from

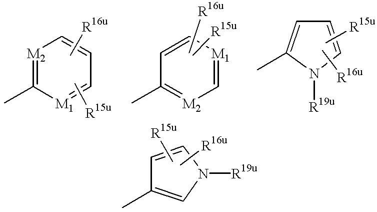

wherein M₁ and M₂ independently are C or N; and

R^{19u} is hydrogen, C₁₋₆-alkyl, phenyl or benzyl; and

R^{15u} is hydrogen, halogen, trifluoromethyl, nitro or cyano; and

R^{16u} is hydrogen, halogen, trifluoromethyl, nitro, cyano, —(CH₂)ₖCOR^{17u}, —(CH₂)ₖOH or —(CH₂)ₖSO₂R^{17u} wherein k is 0, 1 or 2; or

R^{16u} is selected from

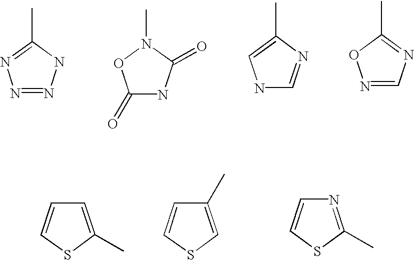

or a pharmaceutically acceptable salt thereof.

Further preferred compounds of the invention include:

1-(3-(10,11-Dihydro-5H-dibenzo[b,f]azepin-5-yl)-(2R)-methyl-1-propyl)-(3R)-piperidinecarboxylic acid;

1-(3-(10,11-Dihydro-5H-dibenzo[b,f]azepin-5-yl)-(2R)-methyl-1-propyl)-4-piperidinecarboxylic acid;

1-(3-(10,11-Dihydro-5H-dibenzo[b,f]azepin-5-yl)-(2R)-methyl-1-propyl)-(2R)-piperidinecarboxylic acid;

1-(4-(10,11-Dihydro-5H-dibenzo[b,f]azepin-5-yl)-(2Z)-butenyl)-(3R)-piperidinecarboxylic acid;

1-(3-(10,11-Dihydro-5H-dibenzo[a,d]cyclohepten-5-ylidene)-1-propionyl)-(3R)-piperidine-carboxylic acid;

1-(2-(10,11-Dihydro-5H-dibenzo[a,d]cyclohepten-5-yl)-1-ethyl)-(3R)-piperidine-carboxylic acid;

1-(4-(10,11-Dihydro-5H-dibenzo[b,f]azepin-5-yl)-(2E)-butenyl)-(3R)-piperidinecarboxylic acid;

1-(2-(10,11-Dihydro-5H-dibenzo[b,f]azepin-5-yl)-1-methyl-1-ethyl)-(3R)-piperidinecarboxylic acid;

1-(3-(10,11-Dihydro-5H-dibenzo[b,f]azepin-5-yl)-2-methyl-3-oxopropyl)-(3R)-piperidinecarboxylic acid;

1-(4-(10,11-Dihydro-5H-dibenzo[b,f]azepin-5-yl)-2-butynyl)-(3R)-piperidinecarboxylic acid;

1-(3-(10,11-Dihydro-5H-dibenzo[b,f]azepin-5-yl)-1-methyl-1-propyl)-(3R)-piperidinecarboxylic acid;

1-(3-(10,11-Dihydro-5H-dibenzo[b,f]azepin-5-yl)-2-hydroxy-1-propyl)-(3R)-piperidinecarboxylic acid;

1-(2-(10,11-Dihydro-dibenzo[b,f]azepin-5-ylmethyl)-1-pentyl)-(3R)-piperidinecarboxylic acid;

1-(3-(3-Chloro-10,11-dihydro-5H-dibenzo[b,f]azepin-5-yl)-(2R)-methyl-1-propyl)-(3R)-piperidinecarboxylic acid;

1-(3-(3-Trifluoromethyl-10,11-dihydro-5H-dibenzo[b,f]azepin-5-yl)-(2R)-methyl-1-propyl)-(3R)-piperidinecarboxylic acid;

1-(3-(3-Methyl-10,11-dihydro-5H-dibenzo[b,f]azepin-5-yl)-(2R)-methyl-1-propyl)-(3R)-piperidinecarboxylic acid;

1-(3-(3-Methoxy-10,11-dihydro-5H-dibenzo[b,f]azepin-5-yl)-(2R)-methyl-1-propyl)-(3R)-piperidinecarboxylic acid;

1-(3-(2-Chloro-10,11-dihydro-5H-dibenzo[b,f]azepin-5-yl)-(2R)-methyl-1-propyl)-(3R)-piperidinecarboxylic acid;

2-(4-(3-(10,11-Dihydro-5H-dibenzo[b,f]azepin-5-yl)-(2R)-methyl-1-propyl)-1-piperazinyl)-nicotinic acid;

1-(2-(10,11-Dihydro-5H-dibenzo[a,d]cyclohepten-5-ylidene)-1-propyl)-(3R)-piperidinecarboxylic acid;

1-(2-(10,11-Dihydro-5H-dibenzo[b,f]azepin-5-yl)-cyclopropylmethyl)-(3R)-piperidinecarboxylic acid;

1-(2-(10,11-Dihydro-5H-dibenzo[b,f]azepin-5-yl)-cyclopentylmethyl)-(3R)-piperidinecarboxylic acid;

1-(2-(10,11-Dihydro-5H-dibenzo[a,d]cyclohepten-5-yl)-1-ethyl)-(3R)-piperidinecarboxylic acid;

(R)-1-(3-(10,11-Dihydro-5H-dibenzo[b,f]azepin-5-yl)-3-oxopropyl)-3-piperidinecarboxylic acid;

(R)-1-(4-(10,11-Dihydro-5H-dibenzo[a,d]cyclohepten-5-yl)-benzyl)-3-piperidinecarboxylic acid;

(R)-1-(4-(10,11-Dihydro-5H-dibenzo[b,f]azepin-5-yl)-2-butyn-1-yi)-3-piperidinecarboxylic acid

(R)-1-((2R)-Methyl-3-(3-methyl-10,11-dihydro-5H-dibenzo[b,f]azepin-5-yl)-1-propyl)-4-piperidinecarboxylic acid;

(R)-1-(3-(10,11-Dihydro-5H-dibenzo[b,f]azepin-5-yl)1-methylpropyl)-3-piperidinecarboxylic acid;

(R)-1-(2-(10,11-dihydro-5H-dibenzo[b,f]azepin-5-yl)-1-methyl-ethyl)-3-piperidinecarboxylic acid;

(R)-1-(2-(10,11-Dihydro-5H-dibenzo[a,d]cyclohepten-5-ylidene)-1-propyl)-3-piperidinecarboxylic acid;

(R)-1-(10,11-Dihydro-5H-dibenzo[a,d]cyclohepten-5-yl)methyl)-3-piperidinecarboxylic acid;

1-(3-(10,11-Dihydro-5H-dibenzo[b,f]azepin-5-yl)-(2R)-methyl-1-propyl)-3-pyrrolidinylacetic acid;

2-(1-(3-(10,11-Dihydrodibenzo[b,f]azepin-5-yl)-(2R)-methylpropyl)-4-piperazinyl)-nicotinic acid;

(R)-1-(2-(10,11-Dihydro-5H-dibenzo[b,f]azepin-5-ylmethyl)-1-pentyl)-3-piperidinecarboxylic acid;

2-(4-(3-(10,11-Dihydro-5H-dibenzo[b,f]azepin-5-yl)-2-hydroxypropyl)piperazin-1-yl)nicotinic acid;

1-(3-(10,11-Dihydro-5H-dibenzo[b,f]azepin-5-yl)-2-methyl-3-oxo-propyl)-3-piperidinearboxylic acid;

(R)-1-(3-(10,11-Dihydro-5H-dibenzo[b,f]azepin-5-yl)-1-propionyl)-3-piperidinecarboxylic acid;

1-(3-(10,11-Dihydro-5H-dibenzo[b,f]azepin-5-yl)-1-propionyl)-4-piperidinecarboxylic acid;

(R)-1-(2-(10,11-Dihydro-5H-dibenzo[b,f]azepin-5-ylcarbonyl)-1-benzyl)-3-piperidinecarboxylic acid;

(R)-1-(2-(10,11-Dihydro-5H-dibenzo[b,f]azepin-5-ylmethyl)-benzyl)-3-piperid inecarboxylic acid;

(R)-1-(3-(10,11-Dihydro-5H-dibenzo[a,d]cyclohepten-5-yl)-3-oxo-1-propyl)-3-piperidinecarboxylic acid;

1-(3-(3-Chloro-10,11-dihydro-5H-dibenzo[b,f]azepin-5-yl)-(2R)-methylpropyl)-4-piperidine-carboxylic acid;

1-(3-(10,11-Dihydro-5H-dibenzo[b,f]azepin-5-yl)-2-hydroxy-propyl)-4-piperidinecarboxylic acid;

(R)-1-(3-(10,11-Dihydro-5H-dibenzo[b,f]azepin-5-yi)-2-hydroxypropyl)-3-piperidinecarboxylic acid;

1-(3-(10,11-Dihydro-5H-dibenzo[b,f]azepin-5-yl)-2-propoxypropyl)-4-piperidinecarboxylic acid;

(R)-1-(2-(N-(10,11-dihydro-5H-dibenzo[a,d]cyclohepten-5-yl)-N-methylamino)ethyl)-3-piperidinecarboxylic acid.

In another preferred embodiment of the invention in formula Ia

R¹, R^{1a}, R² and R^{2a} independently are hydrogen, halogen, trifluoromethyl, hydroxy, C₁l-6alkyl, C₁₋₆-alkoxy or methylthio, —NR⁷R⁸ or —SO₂NR⁷R⁸ wherein R⁷ and R⁸ independently are hydrogen or C₁₋₆-alkyl; and

Y is >CH—O— or >CH—S(O)_{y} wherein y is 0, 1 or 2, or —N(R⁸)— wherein R⁸ is hydrogen or C₁₋₆-alkyl; and

X is completion of an optional bond, ortho-phenylene, —O—, —S—, —C(R⁷R⁸)—, —CH₂CH₂—, —CH═CH—CH₂—, —CH₂—CH═CH—, —CH₂—(C═O)—, —(C═O)—CH₂—, —CH₂CH₂CH₂—, —CH═CH—, —N(R⁸)—(C═O)—, —(C═O)—N(R⁸)—, —O—CH₂—, —CH₂—O—, —OCH₂0—, —CH₂0CH₂—, —S—CH₂—, —CH₂—S—, —(CH₂)N(R⁸)—, —N(R⁸)(CH₂)—, —N(CH₃)SO₂—, —SO₂N(CH₃)—, —CH(R⁹)CH₂—, —CH₂CH(R⁹)—, —(C═O)—, —N(R⁸)— or —(S═O)— wherein R⁷ and R⁸ independently are hydrogen or C₁₋₆-alkyl; and wherein R⁹ is C₁₋₆-alkyl or phenyl; and

p and q independently are 0 or 1; and

r is 1, 2, 3 or4; and

Z is selected from

[chemical structure]

wherein g is 0, 1 or 2; and

R^{11u} is hydrogen, C₁₋₆-alkyl, C₁₋₆-alkoxy or phenyl optionally substituted with halogen, trifluoromethyl, hydroxy, C₁₋₆-alkyl or C₁₋₆-alkoxy; and

R^{12u} is —(CH₂)ₕOH or —(CH₂)ⱼCOR^{17u} wherein h is 0, 1, 2, 3, 4, 5 or 6 and j is 0 or 1 and wherein R^{17u} is —OH, —NHR^{20u} or C₁₋₆-alkoxy wherein R^{20u} is hydrogen or C₁₋₆-alkyl; and

R^{13u} is hydrogen, halogen, trifluoromethyl, hydroxy, C₁₋₆-alkyl or C₁₋₆-alkoxy; and

R^{14u}is hydrogen or C₁₋₆-alkyl; and

C is C₁₋₆-alkylene, C₂₋₆-alkenylene or C₂₋₆-alkynylene; and

[symbol] is optionally a single bond or a double bond; and

R^{18u} is selected from

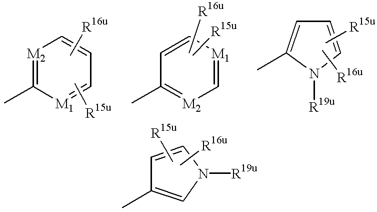

wherein M₁ and M₂ independently are C or N; and

R^{19u} is hydrogen, C₁₋₆-alkyl, phenyl or benzyl; and

R^{15u} is hydrogen, halogen, trifluoromethyl, nitro or cyano; and

R^{16u} is hydrogen, halogen, trifluoromethyl, nitro, cyano, —(CH₂)ₖCOR^{17u}, —(CH₂)ₖOH or —(CH₂)ₖSO₂R^{17u} wherein k is 0, 1 or 2; or

R^{16u} is selected from

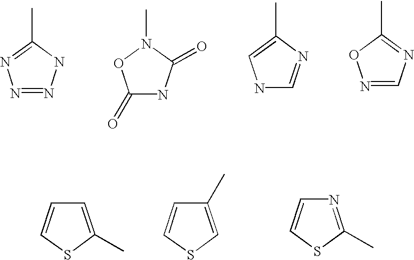

or a pharmaceutically acceptable salt thereof.

Further preferred compounds of the invention include:

1-(2-(10,11-Dihydrodibenzo[b,f]thiepin-10-yloxy)-1-ethyl)-(3R)-piperidinecarboxylic acid;

1-(2-(2-Chloro-10,11-dihydrodibenzo[b,f]thiepin-10-yloxy)-1-ethyl)-3-piperidinecarboxylic acid;

1-(2-(2-Chloro-10,11-dihydrodibenzo[b,f]thiepin-10-yloxy)-1-ethyl)-4-piperidinecarboxylic acid;

1-(2-(2-Methyl-10,11-dihydrodibenzo[b,f]thiepin-10-yloxy)-1-ethyl)-4-piperidinecarboxylic acid;

1-(2-(2-Methyl-10,11-dihydrodibenzo[b,f]thiepin-10-yloxy)-1-ethyl)-3-piperidinecarboxylic acid;

1-(2-(8-Chloro-10,11-dihydrodibenzo[b,f]thiepin-10-yloxy)-1-ethyl)-3-piperidinecarboxylic acid;

1-(2-(8-Methylthio-10,11-dihydrodibenzo[b,f]thiepin-10-yloxy)-1-ethyl)-3-piperidinecarboxylic acid;

(R)-1-(2-(10,11-Dihydrodibenzo[b,f]oxepin-10-yloxy)ethyl)-3-piperidinecarboxylic acid;

(R)-1-(2-(2-Chloro-10,11-dihydrodibenzo[b,f]thiepin-10-ylsulfanyl)ethyl)-3-piperidinecarboxylic acid; (R)-1-(11H-Dibenz[b,f][1 ,4]oxathiepin-11-ylmethyl)-3-piperidinecarboxylic acid; (R)-1-(2-(2-Chloro-7-fluoro-10,11-dihydrodibenzo[b,f]thiepin-10-yloxy)ethyl)-3- piperidinecarboxylic acid; (R)-1-(2-(2,4-Dichloro-10,11-dihydrodibenzo[b,f]thiepin-10-yloxy)ethyl)-3-piperidinecarboxylic acid.

In another preferred embodiment of the invention in formula Ia R¹, R^{1a}, R² and R^{2a} independently are hydrogen, halogen, trifluoromethyl, hydroxy, C₁₋₆-alkyl or Cl.-alkoxy; and Y is >N—CH₂—, >CH—CH₂— or >C═CH— wherein only the underscored atom participates in the ring system; and X is ortho-phenylene, —O—, —S—, —C(R⁷R⁸)—, —CH₂CH₂—, —CH═CH—CH₂—, —CH₂—CH═CH—, —CH₂- (C═O)—, —(C═O)—CH₂—, —CH₂CH₂CH₂—, —CH═CH—, —N(R⁸)—(C═O)—, —(C═o)—N(R⁸)—, —O—CH₂—, —CH₂- O—, —OCH₂0—, —S—CH₂—, —CH₂—S—, —(CH₂)N(R⁸)—, —N(R⁸)(CH₂)—, —N(CH₃)S0₂—, —SO₂N(CH₃)—,- CH(R⁹)CH₂—, —CH₂CH(R⁹)—, —(C═O)—, —N(R⁸)— or —(S═O)— wherein R⁷ and R independently are hydrogen or C,-alkyl; and wherein R⁹ is C,-6alkyl or phenyl; and p and q are 0; and r is 1, 2 or 3; and Z is selected from

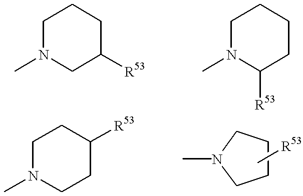

wherein R⁵³ is —(CH₂)ppCOOH wherein pp is 2, 3, 4, 5 or 6; or a pharmaceutically acceptable salt thereof.

Further preferred compounds of the invention include: 3-(1-(3-(10,11-Dihydrodibenzo[a,d]cyclohepten-5-ylidene)-1-propyl)piperidin-3-yl)propionic acid; 3-(1-(3-(10,11-Dihydrodibenzo[b,f]azepin-5-yl)-1-propyl)piperidin-3-yl)propionic acid; 3-(1-(2-(10,11-Dihydrodibenzo[ayd]cyclohepten-5-ylidene)ethyl)piperidin-4-yl)propionic acid; 3-(1-(3-(10,11-Dihydro-5H-dibenzo[a,d]cyclohepten-5-ylidene)-1-propyl)piperidin-4-yl)propionic acid; 3-(1-(3-(10,11-Dihydro-5H-dibenzo[b,f]azepin-5-yl)-1-propyl)piperidin-4-yl)propionic acid; 3-(1-(3-(Thioxanthen-9-ylidene)-1-propyl)piperidin-4-yl)propionic acid; 3-(1-(3-(Xanthen-9-ylidene)-1-propyl)piperidin-4-yl)propionic acid; 3-(1-(3-(12H-Dibenzo[d ,g][1,3]dioxocin-12-ylidene)-1-propyl)piperidin-4-yl)propionic acid; 4-(1-(3-(10,11-Dihydro-5H-dibenzo[a,d]cyclohepten-5-ylidene)-1-propyl)piperidin-4-yl)-butyric acid; 3-(1-(3-(10,11-Dihydro-5H-dibenzo[a,d]cyclohepten-5-ylidene)-1-propyl)piperidin-2-yl)- propionic acid; 3-(1-(3-(1-Bromo-10,11-dihydro-5H-dibenzo[a,d]cyclohepten-5-ylidene)-1-propyl)piperidin-4- yl)propionic acid; 3-(1-(3-(2-Fluoro-10,11-dihydro-5H-dibenzo[a,d]cyclohepten-5-ylidene)-1-propyl)piperidin-4- yl)propionic acid; 3-(1-(3-(2-Trifluoromethyl- 10,11-dihydro-5H-dibenzo[a ,d]cyclohepten-5-ylidene)-1-propyl)- piperidin-4-yl)propionic acid; 3-(1-(3-(2-Hydroxy-10,11-dihydro-5H-dibenzo[a,d]cyclohepten-5-ylidene)-1-propyl)piperidin- 4-yl)propionic acid; 3-(1-(3-(2-Methyl-10,11-dihydro-5H-dibenzo[a,d]cyclohepten-5-ylidene)-1-propyl)piperidin-4- yl)propionic acid; 3-(1-(3-(2-Methoxy-10,11-dihydro-5H-dibenzo[a,d]cyclohepten-5-ylidene)-1-propyl)-piperidin- 4-yl)propionic acid; 5 3-(1-(3-(10,11-Dihydro-5H-dibenzo[a,d]cyclohepten-5-yl)-1-propyl)piperidin-4-yl)propionic acid;

3-(1-(3-(6,11-Dihydro-dibenz[b,e]thiepin-11-ylidene)-1-propyl)piperidin-4-yl)propionic acid; 10 3-(1-(3-(2-Fluoro-6,11-dihydro-dibenz[b,e]thiepin-11-ylidene)-1-propyl)piperidin-4-yl)- propionic acid;

4-(1-(3-(6,11-Dihydro-dibenz[b,e]thiepin-11-ylidene)-1-propyl)piperidin-4-yl)butyric acid; 1 5 3-(1-(3-(6,11-Dihydro-dibenz[b, e]thiepin-1 I 1-ylidene)-1-propyl) pi perid in-3-yl)propion ic acid;

3-(1-(3-(6,11-Dihydro-dibenz[b,e]thiepin-11-ylidene)-1-propyl)piperidin-2-yl)propionic acid;

3-(1l-(3-(l 0,11-Dihydro-5H-dibenzo[a,d]cyclohepten-5-ylidene)-1-propyl)pyrrolidin-3-yl)- propionic acid;

4-(1l-(3-(l 10,11-Dihydro-5H-dibenzo[a ,d]cyclohepten-5-ylidene)-1-pro pyl)pyrrolidin-3-yl) butyric acid; 3-(1-(3-(6,11-Dihydro-dibenz[b,e]thiepin-11-ylidene)-1-propyl)pyrrolidin-3-yl)propionic acid;

3-(1l-(3-(10OH-Anthracen-9-ylidene)-1l-propyl)pyrrolidin-3-yl)propionic acid;

3-(1l-(3-(Dibenzo[a,d]cyclohepten-5-ylidene)-1-propyl)pyrrolidin-3-yl)propionic acid;

3-(1l-(3-(10OH-Anthracen-9-ylidene)-1l-propyl)piperidin-4-yl)propionic acid;

3-(1-(3-(Dibenzo[a,d]cyclohepten-5-ylidene)-1-propyl)piperidin-4-yl)propionic acid; 5-(1-(3-(10,11-Dihydro-5H-dibenzo[a,d]cyclohepten-5-yl1 )-1-propyl)piperdin-4-yl)pentanoi c acid; 5-(1-(3-(6,11-Dihydro-dibenz[b,e]thiepin-11-ylidene)-1-propyl)piperidin-4-yl)pentanoic acid; 5-(1-(3-(Thioxanthen-9-ylidene)-1-propyl)piperidin-4-yl)pentanoic acid; 5-(1-(3-(12H-Dibenzo[d,g][1 ,3]dioxocin-12-ylidene)-1-propyl)piperidin-4-yl)pentanoic acid. In another preferred embodiment of the invention in formula Ia R¹, R^{1a}, R² and R^{2a} independently are hydrogen, halogen, trifluoromethyl, hydroxy, C₁-6alkyl or C₁6-alkoxy; and Y is >N—CH₂—, >CH—CH₂—, >C═CH- or >CH—O— wherein only the underscored atom participates in the ring system; and X is ortho-phenylene, —O—, —S—, —C(R⁷R⁸)—, —CH₂CH₂—, —CH═CH—CH₂—, —CH₂—CH═CH—, —CH₂- (C═O)—, —(C═O)—CH₂—, —CH₂CH₂CH₂—, —CH═CH—, —N(R)—(C═O)—, —(C═O)—N(R⁸)—, —O—CH₂—, —CH₂-O—, —OCH₂0—, —S—CH₂—, —CH₂—S—, —(CH₂)N(R⁸)—, —N(R⁸)(CH₂)—, —N(CH₃)SO₂—, —SO₂N(CH₃)—,-CH(R⁹)CH₂—, —CH₂CH(R⁹)—, —(C═O)—, —N(R3)— or —(S═O)— wherein R⁷ and R⁸ independently are hydrogen or C₁l-6alkyl; and wherein R⁹ is C₁₋₆-alkyl or phenyl; and p and q are 0; and r is 1, 2 or3; and Z is

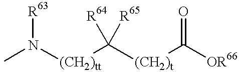

wherein tt and t independently are 0, 1 or 2; and R⁶³ is H, C₁₋₆-alkyl or optionally substituted benzyl; R⁶⁴ and R⁶⁵ independently are H, C₁₈-alkyl, C₃₋₇-cycloalkyl, phenyl, thienyl, benzyl, or R⁶⁴ and R55 together with the C-atom they are attached to form a 3 - 8 membered carbocyclic ring; and R⁶⁶ is H or C₁₋₆-alkyl; or a pharmaceutically acceptable salt thereof. Further preferred compounds of the invention include: 5776-WO,IMSM

1-(2-(10,11-Dihydrodibenzo[b,f]thiepin-10-yloxy)-1-ethyl)-(3R)-piperidinecarboxylic acid;

I -(2-(2-Chloro-10,11-dihydrodibenzo[b,f]thiepin-10-yloxy)-1-ethyl)-3- piperidinecarboxylic acid;

1-(2-(2-Chloro-10,11-dihydrodibenzo[b,f]thiepin-10-yloxy)-1-ethyl)-4- piperidinecarboxylic acid; 1-(2-(2-Methyl-10,11-dihydrodibenzo[b,f]thiepin-10-yloxy)-1-ethyi)-4- piperidinecarboxylic acid;

1-(2-(2-Methyl-10,11-dihydrodibenzo[b,f]thiepin- 10-yloxy)-1-ethyl)-3- piperidinecarboxylic acid;

1-(2-(8-Chloro-10,11-dihydrodibenzo[b,f]thiepin-10-yloxy)-1-ethyl)-3- piperidinecarboxylic acid;

I -(2-(8-Methylthio-10,11-dihydrodibenzo[b,f]thiepin-10-yloxy)-1-ethyl)-3- piperidinecarboxylic acid;

(R)-1-(2-(10,11-Dihydrodibenzo[b,floxepin-10-yloxy)ethyl)-3-piperidinecarboxylic acid;

(R)-1-(2-(2-Chloro-10,11-dihydrodibenzo[b,f]thiepin-10-ylsulfanyl)ethyl)-3- piperidinecarboxylic acid;

(R)-1-(11 H-Dibenz[b,f][1 ,4]oxathiepin-11-ylmethyl)-3-piperidinecarboxylic acid;

(R)-1-(2-(2-Chloro-7-fluoro-10,11-dihydrodibenzo[b,f]thiepin-10-yloxy)ethyl)-3- piperidinecarboxylic acid;

(R)-1-(2-(2,4-Dichloro-10,11-dihydrodibenzo[b,f]thiepin-10-yloxy)ethyl)-3-piperidinecarboxylic acid.

In another preferred embodiment of the invention in formula Ia R¹, R¹a, R² and R^{2a} independently are hydrogen, halogen, trifluoromethyl, hydroxy, C, -alkyl or C₁I-alkoxy; and Y is >N—CH₂—, >CH—CH₂— or >C═CH— wherein only the underscored atom participates in the ring system; and X is ortho-phenylene, —O—, —S—, —C(R⁷R⁸)—, —CH₂CH₂—, —CH═CH—CH₂—, —CH₂—CH═CH—, —CH₂- (C═O)—, —(C═O)—CH₂—, —CH₂CH₂CH₂—, —CH═CH—, —N(R)—(C═O)—, —(C═O)—N(R8)—, -Q—CH₂—, —CH₂- O—, —OCH₂0—, —S—CH₂—, —CH₂—S—, —(CH₂)N(R⁸)—, —N(R⁸)(CH₂)—, —N(CH₃)SO₂—, —SO₂N(CH₃)—, - CH(R⁹)CH₂—, —CH₂CH(R⁹)—, —(C═O)—, —N(R⁸)— or —(S═O)— wherein R⁷ and R⁸ independently are hydrogen or C₁₋₆-alkyl; and wherein R⁹ is Cl-6alkyl or phenyl; and p and q are 0; and r is 0, 1 or 2; and Z is selected from

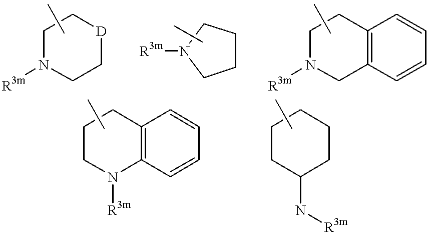

wherein D is —CH₂—, —O—,—S— or —N(R⁷) wherein R⁷ is H or C₁₋₆-alkyl; and R^{3m} is (CH₂)mmOH or (CH₂)mpCOR⁴ wherein mm and mp are 1, 2, 3 or 4 and R⁴ is OH, NH₂, NHOH or C₁₋₆alkoxy; or a pharmaceutically acceptable salt thereof. Further preferred compounds of the invention include: 3-(3-(10,11-Dihydro-5H-dibenzo[b,f]azepin-5-ylmethyl)-pyrrolidin-1-yl)-propionic acid; (2-(2-(l 10,11-Dihydro-5H-dibenzo[b ,flazepin-5-ylmethyl)-morpholin-4-yl)-acetic acid; (3-(10,11-Dihydro-5H-dibenz[(b,flazepin-5-ylmethyl)-1-piperidyl)acetic acid.

In another preferred embodiment of the invention in formula Ia R¹, R^{1a}, R² and R^{2a} independently are hydrogen, halogen, cyano, trifluoromethyl, methylthio, hydroxy, C₁₋₆-alkyl or C₁6-alkoxy; and Y is >N—, >CH—, >N—(C═O)— or >C═C(R8)—, wherein only the underscored atom participates in the ring system and R⁸ is hydrogen or C₁ 6-alkyl; and X is ortho-phenylene, —O—, —S—, —C(R⁷R⁸)—, —CH₂CH₂—, —CH═CH—CH₂—, —CH₂—CH═CH—, —CH₂- (C═O)—, —(C═O)—CH₂—, —CH₂CH₂CH₂—, —CH═CH—, —N(R⁸)—(C═O)—, —(C═O)—N(R⁸)—, —O—CH₂—, —CH₂- O—, —OCH₂O—, —CH₂QCH₂—, —S—CH₂—, —CH₂—S—, —(CH₂)N(R⁸)—, —N(R⁸)(CH₂)—, —N(CH₃)SO₂—, - SO₂N(CH₃)—, —CH(R⁹)CH₂—, —CH₂CH(R⁹)—, —(C═O)—, —N(R⁸)— or —(S═O)— wherein R⁷ and R⁸ independently are hydrogen or C₁₋₆-alkyl; and wherein R⁹ is C₁6-alkyl or phenyl; and p and q are 0; and r is 0, 1, 2, 3 or 4; and Z is

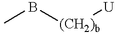

wherein b is 0, 1, 2, 3 or 4; and B is —CH═CR⁴⁹—, —CR⁴⁹═CH—, —C═C—, —(C═O)—, —(C═CH₂)—, —(CR⁴⁹R⁴)—, —CH(OR⁴¹) CH(NHR⁴¹)—, phenylene, C₃₋₇-cycloalkylene or the completion of a bond, wherein R⁴⁹ and R⁴⁰ independently are hydrogen, C₁₋₆-unbranched alkyl, C₃₋₆-branched alkyl or C₃₋₇-cycloalkyl and wherein R⁴¹ is hydrogen or C₁ 6-alkyl; and U is

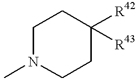

wherein R⁴² is hydrogen, —(CH₂),OH or —(CH₂)dCOR⁴⁷ wherein c is 0, 1, 2, 3, 4, 5 or 6 and d is 0 or 1 and wherein R⁴⁷ is —OH, —NHR⁴⁴ or C₁6-alkoxy wherein R⁴⁴ is hydrogen or C₁₋₆-alkyl; and R43 is cyano, —N R⁴⁵R⁴⁶, —NR⁴⁵-V or —(CH R⁴⁸)eV wherein R⁴⁵ and R⁴⁶ independently are hydrogen or C₁₋₆-alkyl and wherein e is 0, 1, 2, 3, 4, 5 or 6 and wherein R⁴⁸ is hydrogen, halogen, cyano, trifluoromethyl, hydroxy, C₁₋₆-alkyl, C₁₋₆-alkoxy, —NR⁴⁵R⁴⁶ or —COOH, and

wherein V is C₃,-8cycloalkyl, aryl or heteroaryl, which rings may optionally be substituted with one or more halogen, cyano, trifluoromethyl, hydroxy, methylthio, C₁₋₆-alkyl or C₁₋₆-alkoxy; or a pharmaceutically acceptable salt thereof.

Further preferred compounds of the invention include: 1-(3-(10,11-Dihydro-5H-dibenzo[a,d]cyclohepten-5-ylidene)-1-propyl)-4-phenyl-4- piperidinecarboxylic acid; 4-(4-Chlorophenyl)-1-(3-(10,11-dihydro-5H-dibenzo[a,d]cyclohepten-5-ylidene)-1-propyl)-4- piperidinecarboxylic acid; 4-(4-Methylphenyl)-1-(3-(10,11-dihydro-5H-dibenzo[a,d]cyclohepten-5-ylidene)-1-propyl)-4- piperidinecarboxylic acid; 1-(3-(10,11-Dihydro-5H-dibenzo[a,d]cyclohepten-5-ylidene)-1-propyl)-4-anilino-4- piperidinecarboxamide; 2-(1-(3-(10,11-Dihydro-5H-dibenzo[a,d]cyclohepten-5-ylidene)-1-propyl)-4-piperidyl)-2- phenylacetonitrile; 2-(1-(3-(10,11-Dihydro-5H-dibenzo[a,d]cyclohepten-5-ylidene)-1-propyl)-4-piperidinyl)-2- phenylacetic acid; 1-(3-(10,11-Dihydro-5H-dibenzo[a,d]cyclohepten-5-ylidene)-1-propyl)-4-cyano-4 piperidinecarboxylic acid. In another preferred embodiment of the invention in formula Ib Rlb and R²b independently are hydrogen, halogen, trifluoromethyl, hydroxy, C₁₋₆-alkyl or C₁- alkoxy; and R³b is hydrogen or C_{1 3}-alkyl; and Ab is C₁₃-alkylene; and Yb is >CH—CH₂—, >C═CH—, >CH—O—, >C═N—, >N—CH₂— wherein only the underscored atom participates in the ring system; and Zb is selected from

[chemical structure]

wherein nb is 1 or 2; and R^{11b is hydrogen or C}₁₋₆-alkyl; and R1²b is hydrogen, C₁6-alkyl, C₁₋₆-alkoxy or phenyl optionally substituted with halogen, trifluoro- methyl, hydroxy, C₁₋₆-alkyl or C₁₋₆-alkoxy; and R1³b is hydrogen, halogen, trifluoromethyl, hydroxy, C₁₋₆-alkyl or C₁₋₆-alkoxy; and R1⁴b is —(CH₂)mbOH or —(CH₂)tbCOR⁵b wherein mb is 0, 1, 2, 3, 4, 5 or 6 and tb is 0 or 1 and wherein R1⁵b is —OH, NH₂, —NHOH or C₁₋₆-alkoxy; and R⁶b is C₁4alkyl or BbCORl⁵b, wherein Bb is C₁₋₆-alkylene, C₂6-alkenylene or C₂ -alkynylene and R1⁵b is the same as above; and III is optionally a single bond or a double bond; or a pharmaceutically acceptable salt thereof. Further preferred compounds of the invention include: 1-(3-(12H-Dibenzo[d,g][1 ,3]dioxocin-12-ylidene)-1-propyl)-3-piperidinecarboxylic acid; (R)-1-(3-(12H-Dibenzo[d,g][1 ,3]dioxocin-12-ylidene)-1-propyl)-3-piperidinecarboxylic acid; (R)-1-(3-(12H-Dibenzo[d,g][1 ,3]dioxocin-12-ylidene)-1-propyl)-3-piperidinecarboxylic acid ethyl ester; 1-(3-(12H-Dibenzo[d,g][1,3]dioxocin-12-ylidene)-1-propyl)-4-piperidinecarboxylic acid; (R)-1-(3-(2, 1 O-Dichloro-12H-dibenzo[d,g][1 ,3]dioxocin-12-ylidene)-1-propyl)-3- piperidinecarboxylic acid; 1-(3-(12H-Dibenzo[d,g][1 ,3]dioxocin-12-ylidene)-1-propyl)-3-pyrrolidineacetic acid; I -(3-(2 10-Dich loro- I 2H-dibenzo[d, g [1 , 3]d ioxocin- 12-yl ide ne)-1-propyl)-3-pyrrolidineacetic acid; (R)-1-(2-(12H-Dibenzo[d,g][1 ,3]dioxocin-12-yloxy)-1-ethyl)-3-piperidinecarboxylic acid; (R)- 1-(2-(2,10-Dich loro-12H-dibenzo[d, g][1 ,3]dioxocin-12-yloxy)-1-ethyl)-3- piperidinecarboxylic acid; (R)-1-(3-(2-Chloro-12H-dibenzo[d,g][1 ,3,6]dioxazocin-12-yl)-1-propyl)-3-piperidinecarboxylic acid; 1-(3-(12H-Dibenzo[d,g][1 ,3,6]dioxazocin-12-yl)-1-propyl)-4-piperidinecarboxylic acid; 2-Chloro-12-(3-dimethylamino)propylidene-I 2H-dibenzo[d,g][1 ,3]dioxocine; 2,10-Dichloro-12-(2-dimethylamino)ethoxy-12H-dibenzo[d,g][1 ,3]dioxocine; 2,10-Dichloro-12-(3-dimethylamino)propyl-12H-dibenzo[d,g][1 ,3]dioxocine; 2,10-Dichloro-12-(3-dimethylamino-1-methyl)ethoxy-12H-dibenzo[d,g][1 ,3]dioxocine; 3-Chloro-12-(2-dimethylaminopropylidene)-12H-dibenzo[d,g][1 ,3]dioxocine; 3-Chloro-12-(3-dimethylamino)propylidene-12H-dibenzo[d,g][1 ,3]dioxocine; 3-Chloro-12-(3-dimethylamino-1-methylpropylidene)-12H-dibenzo-[d,g][1 ,3]dioxocine; 2-Fluoro-12-(3-dimethylamino)propylidene-12H-dibenzo[d,g][1 ,3]dioxocine; 2-Methyl-12-(3-(4-methyl-1-piperazinyl)propylidene)-12H-dibenzo[d,g][1,3]dioxocine; 2-Chloro-12-(3-(4-methyl-1-piperazinyl)propylidene)-12H-dibenzo[d,g][1 ,3]dioxocine; 3-Chloro-12-(3-(4-methyl-1-piperazinyl)propylidene)-12H-dibenzo[d ,g][1 ,3]dioxocine; 1-(3-(12H-Dibenzo[d,g][1 ,3]dioxocin-12-ylidene)propyl)-3-piperidinecarboxylic acid ethyl ester; 1-(3-(12H-Dibenzo[d ,g][1 ,3]dioxocin-12-ylidene)propyl)-3-piperidinecarboxylic acid. In another preferred embodiment of the invention in formula Ic RlC and R²C independently are hydrogen, halogen, trifluoromethyl, hydroxy, C₁₋₆-alkyl or C₁6- alkoxy; and XC is ortho-phenylene, —O—, —S—, —C(RcRc)—, —CH₂CH₂—, —CH═CH—CH₂—, —CH₂—CH═CH—, —CH₂- (C═O)—, —(C═O)—CH₂—, —CH₂CH₂CH₂—, —CH═CH—, —N(R° C)—(C═O)—, —(C═O)—N(R° C)—, —O—CH₂—, —CH₂- O—, —OCH₂0—, —S—CH₂—, —CH₂—S—, —(CH₂)N(R8c), —N(R8c)(CH₂)—, —N(CH₃)SO₂—, —SO₂N(CH₃)—,- CH(RlOC)CH₂—, —CH₂CH(R¹⁰c)—, —(C═O)—, —N(R⁹c)— or —(S═O)— wherein R⁶C, R⁷C, R° C and R⁹c independently are hydrogen or C₁I-alkyl, and wherein RloC is C₁-6alkyl or phenyl; and Yc is C or N; and I is optionally a single bond or a double bond, and [symbol] is a single bond when Yc is N; and mcis1,2,3,4, 5or6; and ZC is —COOR3c or

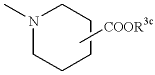

wherein R³c is H or C₁₋₆-alkyl;or a pharmaceutically acceptable salt thereof. Further preferred compounds of the invention include: 1-(2-(10,11-Dihydrodibenzo[b,f]thiepin-10-yloxy)-1-ethyl)-(3R)-piperidinecarboxylic acid; 1-(2-(2-Chloro-10,11-dihydrodibenzo[b,f]thiepin-10-yloxy)-1-ethyl)-3-piperidine- carboxylic acid; I -(2-(2-Ch loro-10,11-dihydrodibenzo[b,f]thiepin-10-yloxy)-1-ethyl)-4-piperidine- carboxylic acid; 1-(2-(2-Methyl-10,11-dihydrodibenzo[b,f]thiepin-10-yloxy)-1-ethyl)-4-piperidine- carboxylic acid; 1-(2-(2-Methyl-10,11-dihyd rodibenzo[b,f]thiepin-10-yloxy)-1-ethyl)-3-piperidine- carboxylic acid; 1-(2-(8-Chloro-10,11-dihydrodibenzo[b,f]thiepin-10-yloxy)-1-ethyl)-3-piperidine- carboxylic acid; 1-(2-(8-Methylthio-10,11-dihydrodibenzo[b,f]thiepin-10-yloxy)-1-ethyl)-3-piperidine- carboxylic acid; (R)-1-(2-(10,11-Dihydrodibenzo[b,qoxepin-10-yloxy)ethyl)-3-piperidinecarboxylic acid; (R)-1-(2-(2-Chloro-10,11-dihydrodibenzo[b,fthiepin-10-ylsulfanyl)ethyl)-3- piperidinecarboxylic acid; (R)-1-(11H-Dibenz[b,fl[1,4]oxathiepin-11-ylmethyl)-3-piperidinecarboxylic acid; (R)-1-(2-(2-Ch loro-7-fluoro- 10,11-dihydrodibenzo[b,f]thiepin-10-yloxy)ethyl)-3- piperidinecarboxylic acid; (R)-1-(2-(2,4-Dichloro-10,11-dihydrodibenzo[b,f]thiepin-10-yloxy)ethyl)-3-piperidinecarboxylic acid. In another preferred embodiment of the invention in formula Id Rld and R²d independently are hydrogen, halogen, trifluoromethyl, hydroxy, C₁₋₆-alkyl or CI- alkoxy; and Xd is —O—, —S— or —S(═O)—; and rd is 0,1,2,3,4,5,6,7,8,9 or 10; and Zd is selected from

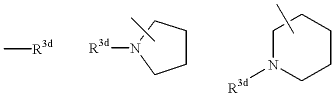

wherein R³d is —(CH₂)mdOH or —(CH₂)pdCOR⁴d wherein md and pd independently are 0, 1, 2, 3 or 4 and R⁴d is OH, NH₂, NHOH or C₁₋₆-alkoxy; or a pharmaceutically acceptable salt thereof.

Further preferred compounds of the invention include:

4-(1 ,3,4,1 4b-Tetrahydro-2H-dibenzo[b,qpyrazino[1 ,2-d][1 ,4]oxazepin-2-yl)-butanoic acid;

4-(1,3,4,14b-Tetrahydro-2H-dibenzo[b,qpyrazino[1,2-d][1,4]thiazepin-2-yl)-butanoic acid.

The compounds of general formulas Ia-Id may be prepared by using the methods taught in WO9631497, WO9631498, WO9631499, WO9631481, WO9711071, WO9815548, WO9815546, WO9815550, PCT/DK98/00273, PCT/DK98/00271, DK 0367/98, DK 0366/98, DK 1472/97 and DK 1523/98, which are hereby incorporated by reference.

It has been demonstrated that the compounds of the present the invention can be used in the treatment of conditions related to angiogenesis according to the following experiment.

### PHARMACOLOGICAL METHODS

The effects of compounds of formulas Ia-Id on angiogenesis are suggested by the following experiments. Air pouches were formed on the dorsum of female To mice and were inflamed one day later by injection of 0.5 ml Freunds complete adjuvant supplemented with 0.1% croton oil. Animals were dosed with compounds of formulas Ia-Id given via the drinking water equivalent to 3-30 mg/kg/day. Control animals received normal drinking water. After 6 days the animals received an injection of carmine in gelatine intravenously prior to dissection of the air pouch granuloma. Comparisons of granuloma dry weight, carmine content and vascular index (carmine content/granuloma dry weight) were made between the groups (Colville-Nash et al., J. Pharmacol. Exp. Ther. 274 1463-1472, 1995).

Treatment with compounds of formulas Ia-Id during 6 days gave reductions in the vascular index between 27-36%

Neovascularization in mouse corneas was induced by surgical implantation of a micropellet containing VEGF (vascular endothelial growth factor) or FGF (fibroblast growth factor) 0.6- 0.8 mm from the corneal limbus. Animals were dosed with compounds of formulas Ia-Id given via the drinking water equivalent to 15 mg/kg/day. After 5 days the stimulation of new blood vessel growth was examined by measuring the vessel length and vessel area (Cao et al., J. Clin. Invest. 98, 2507-2511, 1996).

Treatment with compounds of formulas Ia-Id resulted in a decrease of the vessel area of neovascularization of 30-50%.

### PHARMACEUTICAL COMPOSITIONS

The present invention also relates to pharmaceutical compositions comprising, as an active ingredient, at least one of the compounds according to the invention or a pharmaceutically acceptable salt thereof and, usually, such compositions also contain a pharmaceutically acceptable carrier or diluent.

Pharmaceutical compositions comprising a compound of the present invention may be prepared by conventional techniques, e.g. as described in Remington: The Science and Practise of Pharmacv, 1 gth Ed., 1995. The compositions may appear in conventional forms, for example capsules, tablets, aerosols, solutions, suspensions or topical applications.

Typical compositions include a compound according to the invention or a pharmaceutically acceptable acid addition salt thereof, associated with a pharmaceutically acceptable excipient which may be a carrier or a diluent or be diluted by a carrier, or enclosed within a carrier which can be in the form of a capsule, sachet, paper or other container. In making the compositions, conventional techniques for the preparation of pharmaceutical compositions may be used. For example, the active compound will usually be mixed with a carrier, or diluted by a carrier, or enclosed within a carrier which may be in the form of a ampoule, capsule, sachet, paper, or other container. When the carrier serves as a diluent, it may be solid, semi-solid, or liquid material which acts as a vehicle, excipient, or medium for the active compound. The active compound can be adsorbed on a granular solid container for example in a sachet. Some examples of suitable carriers are water, salt solutions, alcohols, polyethylene glycols, polyhydroxyethoxylated castor oil, syrup, peanut oil, olive oil, gelatine, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavouring agents. The formulations of the invention may be formulated so as to provide quick, sustained, or delayed release of the active ingredient after administration to the patient by employing procedures well known in the art.

The pharmaceutical compositions can be sterilized and mixed, if desired, with auxiliary agents, emulsifiers, salt for influencing osmotic pressure, buffers and/or colouring substances and the like, which do not deleteriously react with the active compounds.

The route of administration may be any route, which effectively transports the active compound to the appropriate or desired site of action, such as oral, nasal, pulmonary, transdermal or parenteral e.g. rectal, depot, subcutaneous, intravenous, intraurethral, intramuscular, topical, intranasal, ophthalmic solution or an ointment, the oral route being preferred.

If a solid carrier is used for oral administration, the preparation may be tabletted, placed in a hard gelatin capsule in powder or pellet form or it can be in the form of a troche or lozenge. If a liquid carrier is used, the preparation may be in the form of a syrup, emulsion, soft gelatin capsule or sterile injectable liquid such as an aqueous or non-aqueous liquid suspension or solution.

For nasal administration, the preparation may contain a compound according to the invention dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet which may be prepared by conventional tablefting techniques may contain:

| Core: | | |
|---|---|---|
| Active compound (as free compound or salt thereof) | 100 | mg |
| Colloidal silicon dioxide (Aerosil) | 1.5 | mg |
| Cellulose, microcryst. (Avicel) | 70 | g |
| Modified cellulose gum (Ac-Di-Sol) | 7.5 | mg |
| Magnesium stearate | | |
| Coating: | | |
| HPMC approx. | 9 | mg |
| *Mywacett 9-40 T approx. | 0.9 | mg |

*Acylated monoglyceride used as plasticizer for film coating.

The compounds of the invention may be administered to a mammal, especially a human in need of such treatment, prevention, elimination, alleviation or amelioration of indications related to angiogenesis. Such mammals include also animals, both domestic animals, e.g. household pets, and non-domestic animals such as wildlife.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, in an effective amount.

The compounds of the invention are effective over a wide dosage range. For example, in the treatment of humans, dosages from about 0.1 to about 1000 mg, preferably from about 0.5 to about 500 mg of compounds of formula 1, conveniently given from I to 5 times daily. A most preferable dosage is from about 50 to about 200 mg per dose when administered to e.g. a human. The exact dosage will depend upon the mode of administration, on the therapy desired, form in which administered, the subject to be treated and the body weight of the subject to be treated, and the preference and experience of the physician or veterinarian in charge.

Generally, the compounds of the present invention are dispensed in unit dosage form comprising from about 50 to about 200 mg of active ingredient in or together with a pharmaceutically acceptable carrier per unit dosage.

Usually, dosage forms suitable for oral, nasal, pulmonal or transdermal administration comprise from about 0.1 mg to about 1000 mg, preferably from about 0.5 mg to about 500 mg of the compounds according to the invention admixed with a pharmaceutically acceptable carrier or diluent.

The method of treating may be described as the treatment, prevention, elimination, alleviation or amelioration of a condition related to angiogenesis in a subject in need thereof, which comprises the step of administering to the said subject an effective amount of a compound of the invention, or a pharmaceutically acceptable salt thereof.

Any novel feature or combination of features described herein is considered essential to this invention.

1. The use of a compound having the general formula Ia 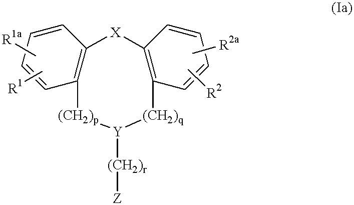
wherein R¹, Ra, R² and R^{2a} independently are hydrogen, halogen, trifluoromethyl, C₁-6alkyl, C₁₋₆-alkoxy, hydroxy, NR⁷R⁸, cyano, methylthio or —SO₂NR⁷R⁸ wherein R⁷and R⁸ independently are hydrogen or C₁₋₆-alkyl; and Y is >N—CH₂- , >CH—CH₂— or >C═CH— wherein only the underscored atom participates in the ring system; or Y is —CH₂N(—)CH₂—, —CHN(—)CH₂—, —(C═O)N(—)CH₂—, —CHgN(—)(C═O)—, —CH;CH(—)CH₂—, - CH₂CH(—)CH₂—, —CH₂C(—)═CH—, —CH═C(—)CH₂—, —OCH(—)CH₂—, —CH₂CH(—)Q—, —SCH(—)CH₂—, - CH₂CH(—)S—, wherein only the underscored atom participates in the ring system; or Y is >N—, >CH—, >N—(C═O)— or >C═C(R⁸)—, wherein only the underscored atom participates in the ring system and R⁸ is hydrogen or C₁₋₆-alkyl; or Y is >CH—O— or >CH—S(O)_{y} wherein y is 0, 1 or 2, or —N(R⁸)— wherein R⁸ is hydrogen or Cl-6alkyl, and wherein only the underscored atom participates in the ring system; and X is completion of an optional bond, ortho-phenylene, —O—, —S—, —C(R⁷R⁸)—, —CH₂CH₂—, —CH═CH- CH₂—, —CH₂—CH═CH—, —CH₂—(C═O)—, —(C═O)—CH₂—, —CH₂CH₂CH₂—, —CH═CH—, —N(R⁸)—(C═O)—, - (C═O)—N(R⁸)—, —O—CH₂—, —CH₂—O—, —OCH₂0—, —CH₂0CH₂—, —S—CH₂—, —CH₂—S—, —(CH₂)N(R⁸)—, - N(R⁸)(CH₂)—, —N(CH₃)SO₂—, —SO₂N(CH₃)—, —CH(R⁹)CH₂—, —CH₂CH(R⁹)—, —(C═O)—, —N(R⁸)— or - (S═O)— wherein R⁷ and R⁸ independently are hydrogen or C₁₋₆-alkyl; and wherein R⁹ is C₁- alkyl or phenyl; and p and q independently are 0 or 1; and r is 0,1, 2, 3 or 4; and Z is selected from 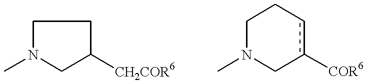
wherein R⁶ is OH or C₁₋₆-alkoxy; and [symbol]
is optionally a single bond or a double bond; or
Z is selected from 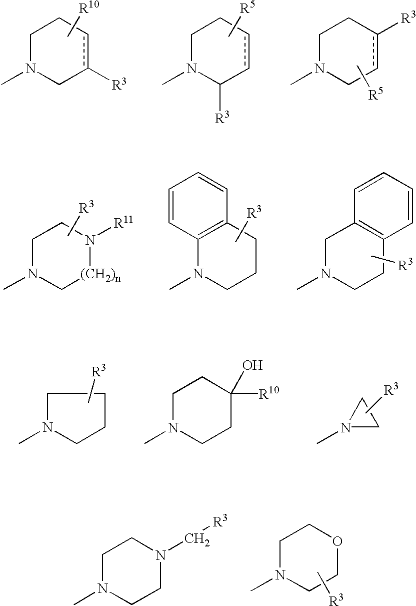
wherein n is 1 or 2;
R3
R4
R5
R10 is hydrogen, C₁₋₆-alkyl, C₁₋₆-alkoxy or phenyl optionally substituted with halogen, trifluoromethyl, hydroxy, C₁₋₆-alkyl or C₁₋₆-alkoxy; and
R11 is hydrogen or C₁₋₆-alkyl; and
z,900 is optionally a single bond or a double bond; or
Z is selected from 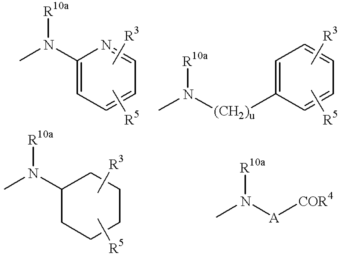
wherein u is 0 or 1; R³ is —(CH₂)ₘOH or —(CH₂)ₛCOR⁴ wherein m is 0, 1, 2, 3, 4, 5 or 6 and s is 0 or 1 and wherein R⁴ is —OH, —NH₂, —NHOH or C₁₋₆-alkoxy; and R⁵ is hydrogen, halogen, trifluoromethyl, hydroxy, C₁₋₆-alkyl or C₁₋₆-alkoxy; and 10 Rloa is hydrogen or C₁6-alkyl; and A is Cl₁6-alkylene, C₂₆-alkenylene or C₂₆-alkynylene; or Z is selected from 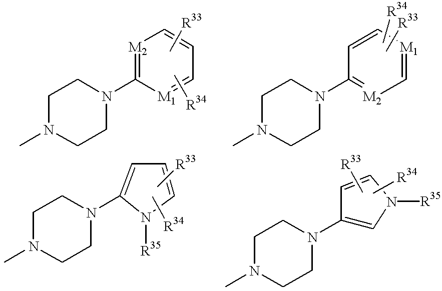
wherein M, and M₂ independently are C or N; and
R³⁵ is hydrogen, C₁₋₆-alkyl, phenyl or benzyl; and
R³³ is hydrogen, halogen, trifluoromethyl, nitro or cyano; and 5 R³⁴ is hydrogen, halogen, trifluoromethyl, nitro, cyano, —(CH₂)_{w}COR³¹4 —(CH₂),OH or - (CH₂)wSO₂R³¹ wherein R³is hydroxy, C₁₋₆-alkoxy or NHR³², wherein R³² is hydrogen or C,l6- alkyl, and w is 0, 1 or 2; or R³⁴ is selected from 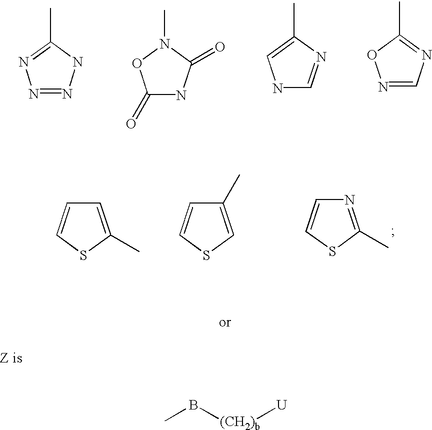
wherein b is 0, 1, 2, 3 or 4; and
B is —CH═CR⁴⁹—, —CR⁴⁹═CH—, —C═C—, —(C═O)—, —(C═CH₂)—, —(CR⁴⁹R⁴⁰)—, —CH(OR⁴)—, - CH(NHR⁴¹)—, phenylene, C₃₋₇-cycloalkylene or the completion of a bond, wherein R⁴⁹ and R⁴⁰ independently are hydrogen, C₁₋₆-unbranched alkyl, C₃₋₆-branched alkyl or C₃₋₇-cycloalkyl and wherein R⁴¹ is hydrogen or C₁₋₆-alkyl; and
U is 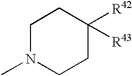
wherein R⁴² is hydrogen, —(CH₂),OH or —(CH₂)dCOR⁴⁷ wherein c is 0, 1, 2, 3, 4, 5 or 6 and d is 0 or 1 and wherein R⁴⁷ is —OH, —NHR⁴⁴ or C₁₋₆-alkoxy wherein R⁴⁴ is hydrogen or C₁₋₆-alkyl;
and
R⁴³ is cyano, —NR⁴⁵R⁴⁷, —N R⁴⁵-V or —(CHR⁴⁸)eV wherein R⁴⁵ and R⁴⁷ independently are hydrogen or C₁6-alkyl and wherein e is 0, 1, 2, 3, 4, 5 or 6 and wherein R⁴⁸ is hydrogen, halogen, cyano, trifluoromethyl, hydroxy, C₁₋₆-alkyl, C₁₋₆-alkoxy, —NR⁴⁵R⁴⁷ or —COOH, and wherein V is C₃₈-cycloalkyl, aryl or heteroaryl, which rings may optionally be substituted with one or more halogen, cyano, trifluoromethyl, hydroxy, methylthio, C₁₋₆-alkyl or C₁₋₆-alkoxy; or U is selected from [chemical structure]
wherein g is 0, 1 or 2; and R^{11u} is hydrogen, C₁6-alkyl, C₁₋₆-alkoxy or phenyl optionally substituted with halogen, trifluoromethyl, hydroxy, C₁₋₆-alkyl or C₁₋₆-alkoxy; and R^{12u} is —(CH₂)ₕOH or —(CH₂)COR 17U wherein h is 0, 1, 2, 3, 4, 5 or 6 and j is 0 or I and wherein R¹⁷U is —OH, —NHR20U or C₁6-alkoxy wherein R^{20u}is hydrogen or C₁₋₆-alkyl; and 5 R^{13u}is hydrogen, halogen, trifluoromethyl, hydroxy, C₁₋₆-alkyl or C₁ 6-alkoxy; and R^{14u}is hydrogen or C₁₋₆-alkyl; and C is C₁6-alkylene, C₂₆-alkenylene or C₂6-alkynylene; and [symbol] is optionally a single bond or a double bond; and R^{18u}is selected from 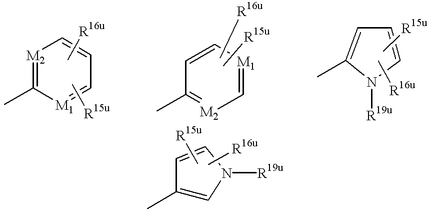
wherein M₁ and M₂ independently are C or N; and R^{19u}is hydrogen, C₁₋₆-alkyl, phenyl or benzyl; and R^{15u}is hydrogen, halogen, trifluoromethyl, nitro or cyano; and R is hydrogen, halogen, trifluoromethyl, nitro, cyano, —(CH₂)kCORI⁷U, —(CH₂)kOH or- (CH₂)kSO₂Rl⁷u wherein k is 0, 1 or 2; or R^{16u} is selected from 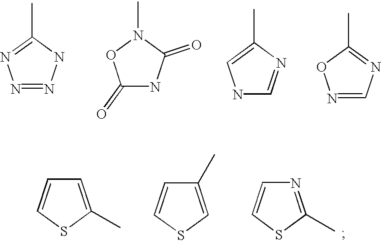
Z is selected from 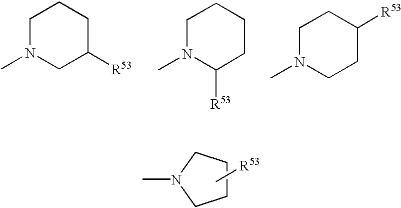
wherein R⁵³ is —(CH₂)ppCOOH wherein pp is 2, 3, 4, 5 or 6; or
Z is 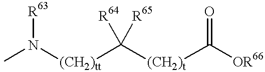
wherein tt and t independently are 0, 1 or 2; and R⁶³ is H, C₁₋₆-alkyl or optionally substituted benzyl; R3⁴ and R⁶⁵ independently are H, C₁₈-alkyl, C₃7-cycloalkyl, phenyl, thienyl, benzyl, or R5⁴ and R⁶⁵ together with the C-atom they are attached to form a 3 - 8 membered carbocyclic ring; and R⁶⁶ is H or C₁₋₆-alkyl; or Z is selected from 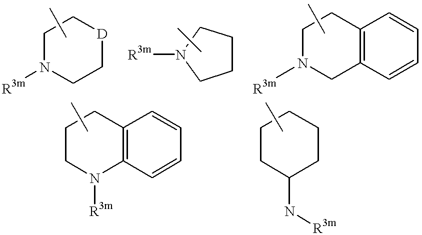
wherein D is —CH₂—, —O—, —S— or —N(R⁷)— wherein R⁷ is hydrogen or C₁₋₆-alkyl; and R³m is —(CH₂)mmOH or —(CH₂)mpCOR⁴ wherein mm and mp are 1, 2, 3 or 4 and R⁴ is OH, NH₂, NHOH or C₁₋₆-alkoxy; or
having the general formula Ib 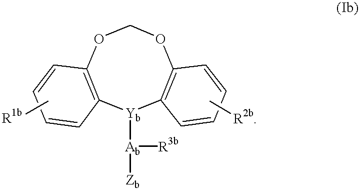
wherein R¹b and R²b independently are hydrogen, halogen, trifluoromethyl, hydroxy, C₁₋₆-alkyl or C₁₋₆-alkoxy; and R³b is hydrogen or CI-3alkyl; and Ab is C₁₃-alkylene; and Yb is >CH—CH₂—, >C═CH—, >CH—O—, >C═N—, >N—CH₂— wherein only the underscored atom participates in the ring system; and Zb is selected from [chemical structure]
wherein nb is 1 or 2; and Rb is hydrogen or C₁₋₆-alkyl; and Rl2b is hydrogen, CI-alkyl, C₁₋₆-alkoxy or phenyl optionally substituted with halogen, trifluoro- methyl, hydroxy, C₁₋₆-alkyl or C₁I-alkoxy; and Rl³b is hydrogen, halogen, trifluoromethyl, hydroxy, Cl-6alkyl or C₁6-alkoxy; and R⁴b is —(CH₂)mbOH or —(CH₂)tbCOR⁵b wherein mb is 0, 1, 2, 3, 4, 5 or 6 and tb is 0 or 1 and wherein R1⁵b is —OH, NH₂, —NHOH or C₁₋₆-alkoxy; and R15b is C₁₋₆-alkyl or -Bb—COR⁵b, wherein Bb is C₁₋₆-alkylene, C₂4-alkenylene or C₂6-alkynylene and Rl⁵b is the same as above; and is optionally a single bond or a double bond; or
having the general formula Ic 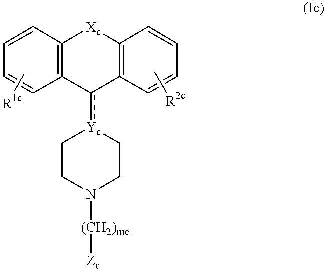
wherein RlC and R²c independently are hydrogen, halogen, trifluoromethyl, hydroxy, C₁6-alkyl or C₁I-alkoxy; XG is ortho-phenylene, —O—, —S—, —C(R^{6c}R^{7c}), —CH₂CH₂—, —CH═CH—CH₂—, —CH₂—CH═CH—, —CH₂- (C═O)—, —(C═O)—CH₂—, —CH₂CH₂CH₂—, —CH═CH—, N(R⁸c)(C═O), (C═O)N(R⁸c), —O—CH₂—, —CH₂- O—, —OCH₂0—, —S—CH₂—, —CH₂—S—, —(CH₂)N(Rc)—, —N(R⁸c)(CH₂)—, —N(CH₃)SO₂—, —SO₂N(CH₃)—,- CH(R^{10c})CH₂—, —CH₂CH(R^{10c})—, —(C═O)—, —N(R⁹c)— or —(S═O)— wherein R6C, R⁷C, Rc and Rc independently are hydrogen or C₁₋₆-alkyl, and wherein R^{10c} is C₁₋₆-alkyl or phenyl; Y_{c} is C or N; [symbol] is optionally a single bond or a double bond, and [symbol] is a single bond when YC is N; mc is 1,2,3,4,5 or 6; and Z_{c} is —COOR^{3c} or 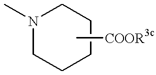
wherein R^{3c} is H or C₁l-alkyl; or
having the general formula Id 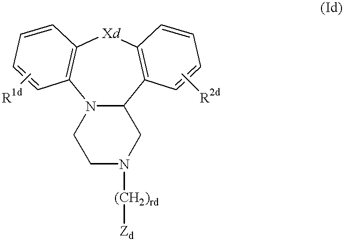
wherein R d and R²d independently are hydrogen, halogen, trifluoromethyl, hydroxy, C₁₋₆-alkyl or C₁₋₆-alkoxy; and Xd is —O—, —S— or —S(═O)—; and rd is 0, 1, 2, 3, 4, 5, 6, 7, 8, 9 or 10; and Zd is selected from 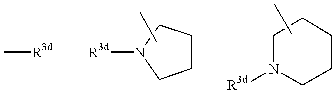
wherein R³d is —(CH₂)mdOH or —(CH₂)pdCOR⁴d wherein md and pd independently are 0, 1, 2, 3 or 4 and R⁴d is OH, NH₂, NHOH or Cl-6alkoxy; or a pharmaceutically acceptable salt thereof, for the manufacture of a pharmaceutical composition for the treatment of an indication related to angiogenesis.

2. The use according to claim 1 wherein angiogenesis is related to cancer.

3. The use according to claim I wherein angiogenesis is related to ocular neovascularization.

4. The use according to anyone of the claims 1-3 wherein in formula Ia ;R¹, R¹, R² and R² independently are hydrogen, halogen, trifluoromethyl, Cl-6alkyl or Cl-6 alkoxy; and Y is >N—CH₂—, >CH—CH₂— or >C═CH— wherein only the underscored atom participates in the ring system; and X is —O—, —S—, —C(R⁷R⁸)—, —CH₂CH₂—, —CH═CH—CH₂—, —CH₂—CH═CH—, —CH₂CH₂CH₂—, —CH═CH—, - N(R⁸)—(C═O)—, —O—CH₂—, —(C═O)— or —(S═O)— wherein R⁷ and R⁸ independently are hydrogen or Cl-6alkyl; and p and q are 0, and r is 1, 2 or 3; and Z is selected from 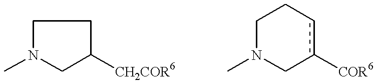
wherein R⁶ is OH or C₁₋₆-alkoxy; and
z,900 is optionally a single bond or a double bond; or
a pharmaceutically acceptable salt thereof.

5. The use according to anyone of the claims 1-4 wherein the compound is selected from the following: (R)-1-(3-(10,11-Dihydro-5H-dibenzo[a,d]cyclohepten-5-ylidene)-1-propyl)-3- piperidinecarboxylic acid; (S)-1-(3-(10,11-Dihydro-5H-dibenzo[a,d]cyclohepten-5-ylidene)-1-propyl)-3- piperidinecarboxylic acid; 1-(3-(10,11-Dihydro-5H-dibenzo[a,d]cyclohepten-5-ylidene)-1-propyl)-1,2,5,6-tetrahydro-3- pyridinecarboxylic acid; (R)-1-(3-(Fluoren-9-ylidene)-1-propyl)-3-piperidinecarboxylic acid; 1-(3-(5H-Dibenzo[a,d]cyclohepten-5-ylidene)-1-propyl)-3-piperidinecarboxylic acid; 1-(3-(Thioxanthen-9-ylidene)-1-propyl)-3-piperid inecarboxylic acid; (R)-1-(3-(10,11-Dihydro-5H-dibenz[b,f]azepin-5-yl)-1-propyl)-3-piperidinecarboxylic acid; (R)-1-(4-(10,11-Dihydro-5H-dibenzo[b,f]azepin-5-yl)-1-butyl)-3-piperidinecarboxylic acid; (R)-1-(2-(10,11-Dihydro-5H-dibenzo[b,qazepin-5-yl)ethyl)-3-piperidinecarboxylic acid; (R)-1-(3-(3-Chloro-10,11-dihydro-5H-dibenzo[b,f]azepin-5-yl)-1-propyi)-3-piperidinecarboxylic acid; (R)-1-(3-(1 OH-Phenothiazin-10-yl)-1-propyl)-3-piperidinecarboxylic acid; (R)-1-(3-(1 OH-Phenoxazin-10-yl)-1-propyl)-3-piperidinecarboxylic acid; (S)-1-(3-(10,11-Dihydro-5H-dibenzo[b,qazepin-5-yl)-1-propyl)-3-piperidinecarboxylic acid; 1-(3-(10,11-Dihydro-5H-dibenzo[b,f]azepin-5-yl)-1-propyl)-3-pyrrolidinacetic acid; (R)-1-(3-(3-Methyl-10,11-dihydro-5H-dibenzo[a,d]cyclohepten-5-ylidene)-1-propyl)-3- piperidinecarboxylic acid;
(R)-1-(3-(2-Trifluoromethyl-10H-phenothiazin-10-yl)-1-propyl)-3-piperidinecarboxylic acid;
(R)-1-(3-(5-Oxo-10H-phenothiazin-10-yl)-1-propyl)-3-piperidinecarboxylic acid;
(R)-1-(3-(11H-10-Oxa-5-aza-5H-dibenzo[a,d]cyclohepten-5-yl)-1-propyl)-3- piperidinecarboxylic acid; 10
1-(3-(10,11-Dihydro-5H-dibenzo[b,f]azepin-5-yl)-1-propyl)-I ,2,5,6-tetrahydro-3- pyridinecarboxylic acid;
(R)-1-(3-(6,7-Dihydro-5H-dibenzo[b,g]azocin-12-yl)-1-propyl)-3-piperidinecarboxylic acid; 1 5
(R)-1-(3-(10,11-Dihydro-5H-dibenzo[a,d]cyclohepten-5-yl)-1-propyl)-3-piperidinecarboxylic acid;
(R)-1-(3-Methoxy-10,11-dihyd ro-5H-dibenzo[b,f]azepin-5-yl)-1-propyl)-3-piperidinecarboxylic 20 acid; 5 (R)-1-(3-(10-Methyl-I I -oxo- 10,11-dihyd ro-5H-dibenzo[b,e][1 ,4]diazepin-5-yl)- I -propyl)-3- piperidinecarboxylic acid; 25 (R)-1-(3-(9(H)-Oxo-1 OH-acridin-10-yl)-1-propyl)-3-piperidinecarboxylic acid; (R)-1-(2-(10,11-Dihydro-5H-dibenzo[a,d]cyclohepten-5-ylidene)-1-ethyl)-3- piperid inecarboxylic acid hydrochloride; 30 (R)-1-(2-(6,11-Dihydrodibenz[b,e]oxepin-11-ylidene)-1-ethyl)-3-piperidinecarboxylic acid hydrochloride; (R)-1-(3-(2-Chloro-10,11-dihydro-5H-dibenzo[a,d]cyclohepten-5-ylidene)-1-propyl)-3- piperidinecarboxylic acid hydrochloride; 35 (R)-1-(3-(2-Bromo-10,11-dihydro-5H-dibenzo[a,d]cyclohepten-5-ylidene)-1-propyl)-3- piperidinecarboxylic acid hydrochloride; (R)-1-(3-(2-Fluoro-10,11-dihydro-5H-dibenzo[a,d]cyclohepten-5-ylidene)-1-propyl)-3- piperidinecarboxylic acid hydrochloride; (R)-1-(3-(2-lodo-10,11-dihyd ro-5H-dibenzo[a ,d]cyclohepten-5-ylidene)-1-propyl)-3- piperidinecarboxylic acid hydrochloride; (Z)-(R)-1-(3-(2-lodo-10,11-dihydro-5H-dibenzo[a,d]cyclohepten-5-ylidene)-1-propyl)-3- piperidinecarboxylic acid hydrochloride; (E)-(R)-1-(3-(2-lodo-10,11-dihydro-5H-dibenzo[a,d]cyclohepten-5-ylidene)-1-propyl)-3- piperidinecarboxylic acid hydrochloride; (R)-1-(3-(2-Methoxy-10,11-dihydro-5H-dibenzo[a,d]cyclohepten-5-ylidene)-1-propyl)-3- piperidinecarboxylic acid hydrochloride, or a pharmaceutically acceptable salt thereof.

6. The use according to anyone of the claims 1-3 wherein in formula Ia R¹, R R² and R^{2a} independently are hydrogen, halogen, trifluoromethyl, hydroxy, C₁-6alkyl or C₁-6alkoxy; and Y is —CH₂N(—)CH₂—, —CH₂N(—)CH₂—, —(C═O)N(—)CH₂—, —CHN(—)(C═O)—, —CH₂CH(—)CH₂—, - C HCH (—)CH₂—, —CH₂C(—)═CH—, —CH═C(—)CH₂—, —CH (—)CH₂—, —CH₂CH (—)O—, —SCH(—)CH₂—, - CH₂CH(—)S—, wherein only the underscored atom participates in the ring system; and X is —O—,—S—, —C(R⁷R⁸)—, —CH₂CH₂—, —CH═CH—CH₂—, —CH₂—CH═CH—, —CH₂—(C═O)—, —(C═O)—CH₂—, - CH₂CH₂CH₂—, —CH═CH—, —N(R⁸)—(C═O)—, —(C═O)—N(R⁸)—, —O—CH₂—, —CH₂—O—, —S—CH₂—, —CH₂—S—, - N(R⁸)—, —(C═O)— or —(S═O)— wherein R⁷ and R⁸ independently are hydrogen or C₁-6alkyl; and p and q independently are 0 or 1; and r is 1, 2or3; and Z is selected from 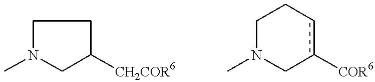
wherein R⁶ is OH or C₁₋₆-alkoxy; and [symbol]
is optionally a single bond or a double bond; or
a pharmaceutically acceptable salt thereof.

7. The use according to anyone of the claims 1-3 and 6 wherein the compound is selected from the following: (R)-1-(3-(6,11-Dioxo-6,1 I -dihydro-5H-dibenz[b,e]azepin-5-yl)-1-propyl)-3- piperidinecarboxylic acid; (R)-1-(3-(6,11-Dihydro-5H-dibenz[b,e]azepin-5-yl)-1-propyl)-3-piperidinecarboxylic acid; (R)-1-(3-(5,11-Dihydro-1 OH-dibenzo[b,e][1 ,4]diazepin-10-yl)-1-propyl)-3-piperidinecarboxylic acid; (R)-1-(3-(11H-Dibenzo[b,fl[1 ,4]thiazepin-10-yl)-1-propyl)-3-piperidinecarboxylic acid; (R)-1-(3-(11 H-Dibenz[b,f][1 ,4]oxazepin-10-yl)-1-propyl)-3-piperidinecarboxylic acid; (R)-1-(3-(11H-Dibenz[b,f][1 ,4]oxathiepin-11-yl)-1-propyl)-3-piperidinecarboxylic acid; (R)-1-(3-(11 H-Dibenzo[b,e][1 ,4]dithiepin-11-yl)-1-propyl)-3-piperidinecarboxylic acid; (R)-1-(3-(1 H-Dibenz[b,e][1,4]oxathiepin-10-yl)-1-propyl)-3-piperidinecarboxylic acid; (R)-1-(3-(11,12-Dihydro-1 OH-dibenz[b,g][1,5]oxazocin-11-yl)-1-propyl)-3-piperidinecarboxylic acid; (R)-1-(3-(11,12-Dihydro-1 OH-dibenzo[b,g][1 ,5]thiazocin-I 1-yl)-1-propyl)-3- piperidinecarboxylic acid; I-(3-(11,12-Dihydro-6H-dibenz[b,qazocin-5-yl)-1-propyl)-3-piperid inecarboxylic acid; 1-(3-(11,12-Dihydro-5H-dibenzo[a,e]cycloocten-5-ylidene)-1-propyl)-3-piperidinecarboxylic acid; I -(3-(6-Oxo-11, 12-dihydro-5H-dibenz[b,f]azocin-5-yl)-1-propyl)-3-piperidinecarboxylic acid; 1-(3-(7,12-Dihydro-6H-dibenzo[a,d]cycloocten-6-ylidene)-1-propyl)-3-piperidinecarboxylic acid; I -(3-(5-Methyl-5, 11-dihydro-dibenz[b,f]azepin-10-ylidene)-1-propyl)-3-piperidinecarboxylic acid; I-(3-(6-Oxo-5,11-dihydro-5H-dibenz[b,e]azepin-5-yl)-1-propyl)-3-piperidinecarboxylic acid; (R)-1-(3-(11-Oxo-10,11-dihydro-5H-dibenzo[b,e][1 ,4]diazepin-10-yl)-1-propyl)-3- piperidinecarboxylic acid; (R)-1-(3-(6-Oxo-11,12-dihydro-5H-dibenz[b,f]azocin-5-yl)- I -propyl)-3-piperidinecarboxylic acid; (R)-I -(3-(I 0,11-Dihydro-dibenz[b,fl[1,4]oxazepin-10-yl)-1-propyl)-3-piperidinecarboxylic acid; (R)-1-(3-(5,6, 11,12-Tetrahydro-dibenz[b,f]azocin-5-yl)-1-propyl)-3-piperidinecarboxylic acid; (R)-1-(3-(11-Oxo-6,1 I -dihydro-5H-dibenz[b,e]azepin-5-yl)-1-propyl)-3-piperidinecarboxylic acid; (R)-I-(3-(5-Methyl-dibenz[b,fqazepin-10-yl)-1-propyl)-3-piperidinecarboxylic acid; (R)-1-(3-(6,7-Dihydro-5H-dibenz[b,g][1 ,5]oxazocin-6-yl)-1-propyl)-3-piperidinecarboxylic acid; (R)-1-(3-(11,12-Dihydro-dibenz[a,e]cycloocten-5-yl)-1-propyl)-3-piperidinecarboxylic acid, or a pharmaceutically acceptable salt thereof.

8. The use according to anyone of the claims 1-3 wherein in formula Ia R¹, R a, R2 and R²independently are hydrogen, halogen, trifluoromethyl, NR⁷R⁸, hydroxy, C, ₆-alkyl or C₁₋₆-alkoxy wherein R⁷ and R⁸ independently are hydrogen or C₁₋₆-alkyl; and Y is >N—CH₂—, >CH—CH₂— or >C═CH— wherein only the underscored atom participates in the ring system; and X is —O—,—S—, —C(R⁷R⁸)—, —CH₂CH₂—, —CH═CH—CH₂—, —CH₂—CH═CH—, —CH₂—(C═O)—, —(C═O)—CH₂—, - CH₂CH₂CH₂—, —CH═CH—, —N(R⁸)(C═O)—, —(C═O)—N(R⁸)—, —O—CH₂—, —CH₂—O—, —S—CH₂—, —CH₂—S—, - N(R⁸)—, —(C═O)— or —(S═O)— wherein R⁷ and R⁸ independently are hydrogen or C₁ -alkyl; and p and q are 0; and r is 1, 2 or3; and Z is selected from 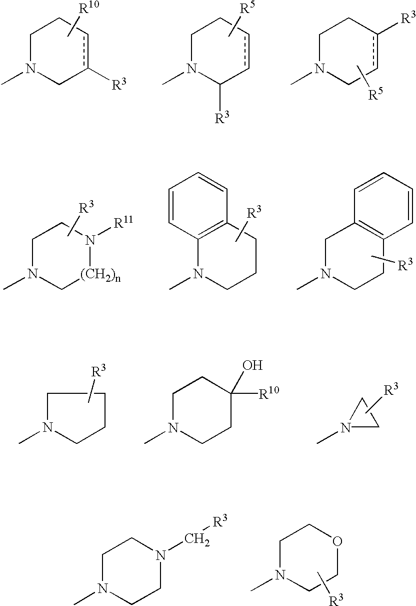
wherein n is 1 or 2; and R³ is —(CH₂)ₘOH or —(CH₂)sCOR⁴ wherein m is 0, 1, 2, 3, 4, 5 or 6 and s is 0 or 1 and wherein R⁴ is —OH, —NH₂, —NHOH or C₁₋₆-alkoxy; and R⁵ is hydrogen, halogen, trifluoromethyl, hydroxy, C₁₋₆-alkyl or C₁₋₆-alkoxy; and R° is hydrogen, C₁₋₆-alkyl, C₁₋₆-alkoxy or phenyl optionally substituted with halogen, trifluoromethyl, hydroxy, C₁₋₆-alkyl or C₁₋₆-alkoxy; and R is hydrogen or C₁₋₆-alkyl; and [symbol] is optionally a single bond or a double bond; or a pharmaceutically acceptable salt thereof.

9. The use according to anyone of the claims 1-3 and 8 wherein the compound is selected from the following: 1-(3-(10,11-Dihydro-5H-dibenz[b,f]azepin-5-yl)-1-propyl)-3-piperidine-carboxamide; 1-(3-(10,11-Dihydro-5H-dibenz[b,f]azepin-5-yl)-i-propyl)-4-piperidinecarboxylic acid; 1-(3-(10,11-Dihydro-5H-dibenz[b,f]azepin-5-yl)-1-propyl)-2-piperidinecarboxylic acid; (1-(3-(10,11-Dihydro-5H-dibenz[b,f]azepin-5-yl)-1-propyl)-3-piperidinyl)methanol; 4-(4-Chlorophenyl)- 1-(3-(10,11-dihydro-5H-dibenz[b,f]azepin-5-yl)-1-propyl)-4-piperidinol; 4-(3-(10,11-Dihydro-5H-dibenz[b,f]azepin-5-yl)-1-propyl)-2-piperazinecarboxylic acid; (2S ,4R)-1-(3-(10,11-Dihydro-5H-dibenz[b,f]azepin-5-yl)-1-propyl)-4-hydroxy-2- pyrrolidinecarboxylic acid; 4-(3-(10,11-Dihydro-5H-dibenz[b,f]azepin-5-yl)-1-propyl)-2-morpholinecarboxylic acid; 1- (3-(10,11-Dihydro-5H-dibenz[b,f]azepin-5-yl)-1-propyl)-2-aziridinecarboxylic acid; 2-(3-(10,11-Dihydro-5H-dibenz[b,f]azepin-5-yl)-1-propyl)-1 ,2,3,4-tetrahydro-4- isoquinolinecarboxylic acid; 1-(3-(10,11-Dihydro-5H-dibenz[b,f]azepin-5-yl)-1-propyl)-4-methyl-[1 ,4]-diazepane-6- carboxylic acid; 2-(3-(10,11-Dihydro-5H-dibenz[b,f]azepin-5-yl)-1-propyl)-1 ,2,3,4-tetrahydro-3- isoquinolinecarboxylic acid; 1-(3-(10,11-Dihydro-5H-dibenz[b,f]azepin-5-yl)-1-propyl)-3-piperidinecarboxylic acid hydroxamide; (4-(3-(10,11-Dihydro-5H-dibenz[b,f]azepin-5-yl)-1-propyl)piperazin-1-yl)acetic acid; 1-(3-(10,11-Dihydro-5H-dibenz[b,f]azepin-5-yl)-1-propyl)-4-piperidinecarboxylic acid; 4-(3-(10,11-Dihydro-5H-dibenz[b,f]azepin-5-yl)-1-propyl)-2-piperazinecarboxylic acid; I-(3-(10,11-Dihydro-5H-dibenz[b,f]azepin-5-yl)-1-propyl)-4-piperidineacetic acid; 1-(3-(10,11-Dihydro-5H-dibenzo[a,d]cyclohepten-5-ylidene)-1-propyl)-4-piperidinecarboxylic acid; (R)-1-(3-(10,11-Dihydro-5H-dibenzo[a,d]cyclohepten-5-ylidene)-1-propyl)-3- piperidinecarboxamide; (R)-1-(3-(10,11-Dihydro-5H-dibenzo[a,d]cyclohepten-5-ylidene)-1-propyl)-2- pyrrolidinecarboxylic acid; (S)-1-(3-(10,11-Dihydro-5H-dibenzo[a,d]cyclohepten-5-ylidene)-1-propyl)-2- pyrrolidinecarboxylic acid; 1-(3-(10,11-Dihydro-5H-dibenzo[a,d]cyclohepten-5-ylidene)-1-propyl)-2-piperidinecarboxylic acid; 1-(3-(1 OH-Phenoxazin-10-yl)-1-propyl)-4-piperidinecarboxylic acid; 1-(3-(3-Chloro-10,11-dihydro-5H-dibenz[b,f]azepin-5-yl)-1-propyl)-4-piperidinecarboxylic acid; 1-(3-(10,11-Dihydro-5H-dibenz[b,f]azepin-5-yl)-1-propyl)-3-piperid ineacetic acid; 1-(3-(10,11-Dihydro-5H-dibenz[b,f]azepin-5-yl)-1-propyl)-2-methyl-3-piperidinecarboxylic acid; 1-(3-(10,11-Dihydro-5H-dibenz[b,f]azepin-5-yl)-1-propyl)-3-quinuclidiniumcarboxylate; 1-(3-(2,8-Dibromo-10,11-dihydro-5H-dibenz[b,f]azepin-5-yl)-1-propyl)-4-piperidinecarboxylic acid; 1-(3-(3,7-Dichloro-10,11-dihydro-5H-dibenz[b,f]azepin-5-yl)-1-propyl)-4-piperidinecarboxylic acid; 1-(3-(3-Methyl-10,11-dihydro-5H-dibenzo[a,d]cyclohepten-5-ylidene)-1-propyl-4- piperidinecarboxylic acid; 1-(3-(3,7-Dimethyl-10,11-dihydro-5H-dibenz[b,f]azepin-5-yl)-1-propyl)-4-piperidinecarboxylic acid; 1-(3-(3-Dimethylamino-10,11-dihydro-5H-dibenz[b,f]azepin-5-yl)-1-propyl)-4-piperidine- carboxylic acid; (R)-1-(3-(10,11-Dihydro-5H-dibenzo[a,d]cyclohepten-5-ylidene)-1-propyl)-2- piperidinecarboxylic acid; (S)-1-(3-(10,11-Dihydro-5H-dibenzo[a,d]cyclohepten-5-ylidene)-1-propyl)-2- piperidinecarboxylic acid; 1-(2-(6,11-Dihydrodibenzo[b,e]thiepin-11-ylidene)-1-ethyl)-3-piperidinecarboxylic acid; 1-(2-(6,11-Dihydrodibenzo[b,e]thiepin-11-ylidene)-1-ethyl)-4-piperidinecarboxylic acid; 1-(2-(2-Chloro-6,11-dihydrodibenzo[b,e]thiepin-11-ylidene)-1-ethyl)-3-piperidinecarboxylic acid; 1-(2-(2-Chloro-6,11-dihydrodibenzo[b,e]thiepin-11-ylidene)-1-ethyl)-4-piperidinecarboxylic acid; (R)-1-(2-(6,11-Dihydrodibenzo[b,e]thiepin-11-ylidene)-1-ethyl)-3-piperidinecarboxylic acid; I-(3-(2-Bromo-10,11-dihydro-5H-dibenzo[a,d]cyclohepten-5-ylidene)-1-propyl)-3- pyrrolidineacetic acid; 1-(3-(3-Methyl-10,11-dihydro-5H-dibenzo[a,d]cyclohepten-5-ylidene)-1-propyl)-3- pyrrolidineacetic acid; 1-(3-(6,11-Dihydro-dibenz[b,e]thiepin-11-ylidene)-1-propyl)-4-piperidinecarboxylic acid; 1-(3-(2-Fluoro-10,11-dihydro-5H-dibenzo[a,d]cyclohepten-5-ylidene)-1-propyl)-4- piperidinecarboxylic acid; 1-(3-(10,11-Dihydro-5H-dibenzo[b,fqazepin-5-yl)-1-propyl)-2-piperidineacetic acid; 1-(3-(Phenothiazin-10-yl)-1-propyl)-4-piperidinecarboxylic acid; (R)-1-(2-(10,11-dihydro-5H-dibenzo[a,d]cyclohepten-5-ylidene)-1-ethyl)-2- piperidinecarboxylic acid; 1-(2-(10,11-Dihydro-5H-dibenzo[a,d]cyclohepten-5-ylidene)-1-ethyl)-4-piperidinecarboxylic acid; 1-(2-(6,11-Dihydrodibenzo[b,e]oxepin-11-ylidene)-1-ethyl)4-piperidinecarboxylic acid, or a pharmaceutically acceptable salt thereof.

10. The use according to anyone of the claims 1-3 wherein in formula Ia R¹, R¹ , R² and R^{2a} independently are hydrogen, halogen, trifluoromethyl, hydroxy, C₁₋₆-alkyl or C,-alkoxy; and Y is >N—CH₂—, >CH—CH₂— or >C═CH— wherein only the underscored atom participates in the ring system; and X is ortho-phenylene, —CH₂—(C═O)—, —(C═O)—CH₂—, —S—CH₂—, —CH₂—S—, —(CH₂)N(R⁸)—, —N(R⁸)(CH₂)- —N(CH₃)S0₂—, -S0₂N(CH₃)—, —CH(R⁹)CH₂— or —CH₂CH(R⁹)— wherein R⁸ is hydrogen or C₁₋₆-alkyl and R⁹ is Cl-6alkyl or phenyl; and p and q are 0; and r is 1,2 or 3; and Z is selected from 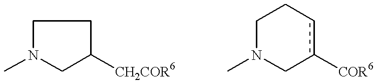
wherein R⁶ is OH or C₁₋₆-alkoxy; and [symbol] is optionally a single bond or a double bond; or a pharmaceutically acceptable salt thereof.

11. The use according to anyone of the claims 1-3 and 10 wherein the compound is selected from the following: 1-(3-(9H-Tribenz[b,d,flazepin-9-yl)-1-propyl)-3-piperidinecarboxylic acid; 1-(3-(Tribenzo[a,c,e]cyclohepten-9-ylidene)-1-propyl)-3-piperidinecarboxylic acid; 1-(3-(5-Methyl-5,6-dihydrodibenz[b,e]azepin-11-ylidene)-1-propyl)-3-piperidinecarboxylic acid; 1-(3-(6-Methyl-6H-dibenzo[c,f][1 ,2]thiazepin-5,5-dioxide-11-ylidene)-1-propyl)-3- piperidinecarboxylic acid; 1- -(3-(10-Methyl-10,11-dihydro-5H-dibenzo[b,e]cyclohepten-5-ylidene)-1-propyl)-3- piperidinecarboxylic acid; 1-(3-(10-Phenyl-10,11-dihydro-5H-dibenzo[b, e]cyclohepten-5-ylidene)-1-propyl)-3- piperidinecarboxylic acid; 1-(3-(6,11-Dihydro-1 I H-dibenzo[b,e][1 ,4]thiazepin-11-yl)-1-propyl)-3-piperidinecarboxylic acid; 1-(3-(10-Methyl-10,11-dihydro-dibenzo[b,e][1 ,4]diazepin-5-yl)-1-propyl)-3- piperidinecarboxylic acid; (R)-1-(3-(10-Oxo-10,11-dihydro-5H-dibenz[b,f]azepin-5-yl)-1-propyl)-3-piperidinecarboxylic acid; (R)-1-(3-(6-Methyl-6,11-dihydro-dibenzo[c,fl[1 ,2,5]thiadiazepin-5,5-dioxide-11-yl)-1-propyl)-3- piperidinecarboxylic acid; (R)-1-(3-(5-Methyl-5,6-dihydrodibenz[b,e]azepin-11-ylidene)-1-propyl)-3-piperidinecarboxylic acid; (R)-1-(3-(9H-Tribenzo[a,c,e]cyclohepten-9-ylidene)-1-propyl)-3-piperidinecarboxylic acid; (R)-1-(3-(9H-Tribenzo[b,d,flazepine-9-yl)propyl)-3-piperidinecarboxylic acid, or a pharmaceutically acceptable salt thereof.

12. The use according to anyone of the claims 1-3 wherein in formula Ia R¹, R¹ , R² and R^{2a} independently are hydrogen, halogen, trifluoromethyl, hydroxy, C₁₋₆-alkyl or C,-6alkoxy; and Y is >N—CH₂—, >CH—CH₂— or >C═CH— wherein only the underscored atom participates in the ring system; and X is —O—, —S—, —C(R⁷R⁸)—, —CH₂CH₂—, —CH═CH—CH₂—, —CH₂—CH═CH—, —CH₂—(C═O)—, —(C═O)—CH₂—, - CH₂CH₂CH₂—, —CH═CH—, —N(R⁸)—(C═O)—, —(C═O)—N(R⁸)—, —O—CH₂—, —CH₂—O—, —S—CH₂—, —CH₂—S—, - N(R⁸)—, —(C═O)— or —(S═O)— wherein R⁷ and R⁸ independently are hydrogen or C₁₋₆-alkyl; and p and q are 0; and r is 1, 2 or 3; and Z is selected from 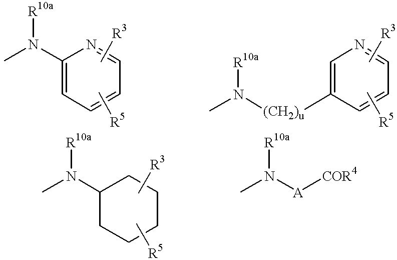
wherein u is 0 or 1;
R³ is —(CH₂)ₘOH or —(CH₂),CoR⁴ wherein m is 0, 1, 2, 3, 4, 5 or 6 and s is 0 or 1 and wherein R⁴ is —OH, —NH₂, —NHOH or C₁₋₆-alkoxy; and R⁵ is hydrogen, halogen, trifluoromethyl, hydroxy, C₁₋₆-alkyl or C₁₋₆-alkoxy; and
ROa is hydrogen or C₁₋₆-alkyl; and
A is C₁₋₆-alkylene, C₂6-alkenylene or C₂6-alkynylene; or a pharmaceutically acceptable salt thereof.

13. The use according to anyone of the claims 1-3 and 12 wherein the compound is selected from the following:
3-(N-Methyl-N-(3-(10,11-dihydrodibenzo[a,d]cyclohepten-5-ylidene)-1-propyl)amino)propionic acid; 4-(N-Methyl-N-(3-(10,11-dihydrodibenzo[a,d]cyclohepten-5-ylidene)-1-propyl)amino)butyric acid; 3-((3-(10,11-Dihydro-5H-dibenz[b,f]azepin-5-yl)-1-propyl)amino)propionic acid; 20 2-(N(3-(, 0,11-Dihydro-5H-dibenz[b,f]azepin-5-yi)-1-propyl)-N-methyl-amino)succinic acid; 2-((3-(10,11-Dihydro-5H-dibenz[b,f]azepin-5-yl)-1-propyl) amino)benzoic acid; 25 2-(N-(3-(10,11-Dihydro-5H-dibenz[b,f]azepin-5-yl)-1-propyl)-N-methylamino)nicotinic acid; 2-((N-(3-(10,11-Dihydro-5H-dibenz[b,f]azepin-5-yi)-1-propyl)-N-methylamino)methyl)benzoic acid; 30 2-((N-(3-(10,11-Dihydro-5H-dibenz[b,f]azepin-5-yl)-1-propyl)-N-methylamino)-1- cyclohexanecarboxylic acid; 2-(3-(10,11-Dihydro-5H-dibenz[b,f]azepin-5-yl)-1-propylamino)pyridin-3-ol; 35 3-((3-(10,11-Dihydro-5H-dibenz[b,f]azepin-5-yl)-1-propyl)amino)benzoic acid; 2-((3-(10,11-Dihydro-5H-dibenzo[a,d]cyclohepten-5-ylidene)-1-propyl)amino)benzoic acid; 2-(N-(3-(3-Chloro-10,11-dihydro-5H-dibenz[b,f]azepin-5-yl)-1-propyl)amino)benzoic acid; 5-Bromo-2-(N-(3-(10,11-dihydro-5H-dibenzo[a,d]cyclohepten-5-ylidene)-1- propyl)amino)benzoic acid, or a pharmaceutically acceptable salt thereof.

14. The use according to anyone of the claims 1-3 wherein in formula Ia R¹, R^{1a}, R² and R^{2a} independently are hydrogen, halogen, trifluoromethyl, hydroxy,C₁₋₆- 1-6alkyl or Cl-6alkoxy; Y is >N—CH₂—, >CH—CH₂—, >C═CH- or >CH—O- wherein only the underscored atom participates in the ring system; and X is ortho-phenylene, —O—, —S—, —C(R⁷R⁸)—, —CH₂CH₂—, —CH═CH—CH₂—, —CH₂—CH═CH—, —CH₂- (C═O)—, —(C═O)—CH₂—, —CH₂CH₂CH₂—, —CH═CH—, —N(R⁸)—(C═O)—, —(C═O)—N(R⁸)—, —O—CH₂—, —CH₂- O—, —OCH₂0—, —S—CH₂—, —CH₂—S—, —(CH₂)N(R⁸)—, —N(R⁸)(CH₂)—, —N(CH₃)SO₂—, —SO₂N(CH₃)—, - CH(R⁹)CH₂—, —CH₂CH(R9)—, —(C═O)—, —N(R⁸)— or —(S═O)— wherein R⁷ and R⁸ independently are hydrogen or C₁.-alkyl; and wherein R⁹ is C₁₋₆-alkyl or phenyl; and p and q are 0; and r is 1, 2 or 3; and Z is selected from 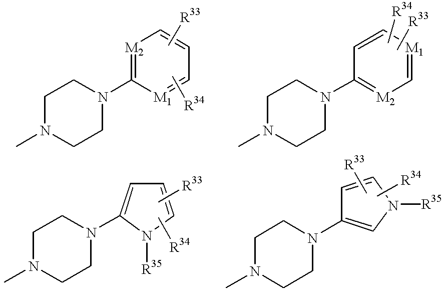
wherein M₁ and M₂ independently are C or N; and R³⁵ is hydrogen, C₁₋₆-alkyl, phenyl or benzyl; and R³³ is hydrogen, halogen, trifluoromethyl, nitro or cyano; and R³⁴ is hydrogen, halogen, trifluoromethyl, nitro, cyano, —(CH₂)ₛCOR³¹ —(CH₂),OH or - (CH₂)wSO₂R³¹ wherein R³is hydroxy, C₁₋₆-alkoxy or NHR³², wherein R³² is hydrogen or C₁4- alkyl, and w is 0, 1 or 2; or R³⁴ is selected from 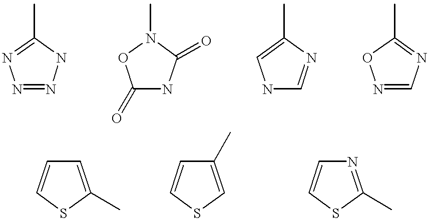
or a pharmaceutically acceptable salt thereof.

15. The use according to anyone of the claims 1-3 and 14 wherein the compound is selected from the following: 2-(4-(3-(12H-Dibenzo[d,g][1 ,3]dioxocin-12-ylidene)-1-propyl)piperazin-1-yl)-3- pyridinecarboxylic acid; 2-(4-(3-(2,10-Dichloro-12H-dibenzo[d,g][1 ,3]dioxocin-12-ylidene)-1-propyl)-piperazin-1- yl)-3-pyridinecarboxylic acid; 2-(4-(3-(12H-Dibenzo[d,g][1 ,3,6]dioxazocin-12-yl)-1-propyl)piperazin-1-yl)-3- pyridinecarboxylic acid; 2-(4-(3-(2-Chloro-12H-dibenzo[d,g][1 ,3,6]dioxazocin-12-yl)-1-propyl)-piperazin-1-yl)-3- pyridinecarboxylic acid; 1-(3-(10,11-Dihydro-5H-dibenzo[a,d]cyclohepten-5-ylidene)-1-propyl)-4-(2- pyridyl)piperazine; 2-(4-(3-(10,11-Dihydro-5H-dibenzo[a,d]cyclohepten-5-ylidene)-propyl)-1-piperazinyl)-3- pyridine-carboxylic acid; 2-(4-(2-(10,11-Dihydro-5H-dibenz[b,f]azepin-5-yl)-1-ethyl)-1-piperazinyl)-3- pyridinecarboxylic acid; 6-(4-(3-(10,11-Dihydro-5H-dibenz[b,f]azepin-5-yl)-1-propyl)-1-piperazinyl)-2- pyridinecarboxylic acid; 2-(4-(3-(10,11-Dihydro-5H-dibenz[b,f]azepin-5-yl)-1-propyl)-1-piperazinyl)-3- pyridinecarboxylic acid; 2-(4-(3-(10,11-Dihydro-5H-dibenzo[b,f]azepin-5-yl)-1-propyl)-1-piperazinyl)-5- pyridinecarboxylic acid; 2-(4-(3-(3-Chloro-10,11-dihydro-5H-dibenzo[b,f]azepin-5-yl)-1-propyl)-1-piperazinyl)3- pyridinecarboxylic acid; 1-(3-(10,11-Dihydro-5H-dibenzo[a,d]cyclohepten-5-ylidene)-1-propyl)-4-(2-nitrophenyl)- piperazine; 2-(4-(3-(10,11-Dihydro-5H-dibenzo[a,d]cyclohepten-5-ylidene)-1-propyl)-1-piperazinyl)- benzonitrile; 2-(4-(3-(10,11-Dihydro-5H-dibenzo[a,d]cyclohepten-5-ylidene)-1-propyl)-1-piperazinyl)- benzoic acid; 1-(3-(10,11-Dihydro-5H-dibenzo[a,d]cyclohepten-5-ylidene)-1-propyl)-4-(3-trifluoromethyl-2- pyridyl)piperazine; 2-(4-(2-(6,11-Dihydro-dibenzo[b,e]thiepin-11-ylidene)ethyl)piperazin-1-yl)-3- pyridinecarboxylic acid; 2-(4-(3-(6,11-Dihydrodibenzo[b,e]thiepin-11-ylidene)-1-propyl)-1-piperazinyl)-3- pyridinecarboxylic acid; 2-(4-(2-(6,11-Dihydrodibenzo[b,e]thiepin-11-yloxy)ethyl)-1-piperazinyl)-3-pyridinecarboxylic acid; 6-(4-(3-(10,11-Dihydro-5H-dibenzo[a,d]cyclohepten-5-ylidene)-1-propyl)piperazin-1-yl)-2- pyridinecarboxylic acid; 2-(4-(3-(3-Methyl-10,11-dihydro-5H-dibenzo[b,f]azepin-5-yl)-1-propyl)-1-piperazinyl)-3- pyridinecarboxylic acid; 6-(4-(3-(Dibenzo[d,g][1 ,3,6]dioxazocin-12-yl)-1-propyl)-piperazin-1-yl)-pyridine-2-carboxylic acid, or a pharmaceutically acceptable salt thereof.

16. The use according to anyone of the claims 1-3 wherein in formula Ia R¹, R R² and R^{2a} independently are hydrogen, halogen, trifluoromethyl, hydroxy, C₁₋₆-alkyl or C₁₋₆-alkoxy; and Y is >N—, >CH—, >N—(C═O)— or >C═C(R⁸)—, wherein only the underscored atom participates in the ring system and R⁸ is hydrogen or CI-alkyl; and X is ortho-phenylene, —O—, —S—, —C(R⁷R⁸)—, —CH₂CH₂—, —CH═CH—CH₂—, —CH₂—CH═CH—, —CH₂- (C═O)—, —(C═O)—CH₂—, —CH₂CH₂CH₂—, —CH═CH—, —N(R⁸)—(C═O)—, —(C═O)—N(R⁸)—, —O—CH₂—, —CH₂- O—, —OCH₂0—, —CH₂0CH₂—, —S—CH₂—, —CH₂—S—, —(CH₂)N(R⁸)—, —N(R⁸)(CH₂)—, —N(CH₃)SO₂—, - SO₂N(CH₃)—, —CH(R⁹)CH₂—, —CH₂CH(R⁹)—, —(C═O)—, —N(R⁸)— or —(S═O)— wherein R⁷ and R⁸ independently are hydrogen or C,-6alkyl; and wherein R⁹ is C₁₋₆-alkyl or phenyl; and p and q are 0; and r is 0, 1, 2, 3 or4; and Z is 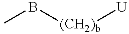
wherein b is 0, 1, 2, 3 or 4; and
B is —CH═CR⁴⁹—, —CR⁴⁹═CH—, —C═C—, —(C═O)—, —(C═CH₂)—, —(CR⁴⁹R⁴⁰)—, —CH(OR⁴¹)—, - CH(NHR⁴¹)—, phenylene, C₃₋₇-cycloalkylene or the completion of a bond, wherein R⁴⁹ and R⁴⁰ independently are hydrogen, C₁₋₆-unbranched alkyl, C₃6-branched alkyl or C₃₋₇-cycloalkyl and wherein R⁴¹ is hydrogen or C₁₋₆-alkyl; and U is selected from [chemical structure]
wherein g is 0, 1 or 2; and R^{11u} is hydrogen, C₁₋₆-alkyl, C₁₋₆- 1alkoxy or phenyl optionally substituted with halogen, trifluoromethyl, hydroxy, C₁₋₆-alkyi or C₁₋₆-alkoxy; and R^{12u} is —(CH₂)ₕOH or —(CH₂)ⱼCOR^{17u} wherein h is 0, 1, 2, 3, 4, 5 or 6 and j is 0 or 1 and wherein R^{17u} is —OH, NHR^{20u} or C₁₋₆-alkoxy wherein R^{20u} is hydrogen or C₁₋₆-alkyl; and R^{13u}is hydrogen, halogen, trifluoromethyl, hydroxy, C₁₋₆-alkyl or C₁₋₆-alkoxy; and R^{14u} is hydrogen or C₁₋₆-alkyl; and C is C₁₋₆-alkylene, C₂₋₆-alkenylene or C₂₋₆-alkynylene; and [symbol]
is optionally a single bond or a double bond; and R^{18u}is selected from 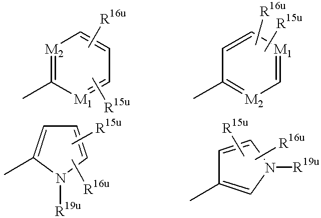
wherein M₁ and M₂ independently are C or N; and R^{19u}is hydrogen, C₁₋₆-alkyl, phenyl or benzyl; and R^{15u}is hydrogen, halogen, trifluoromethyl, nitro or cyano; and R^{16u}is hydrogen, halogen, trifluoromethyl, nitro, cyano, —(CH₂)kCoRl⁷U, —(CH₂)kOH or- (CH₂)ₖSO₂R^{17u} wherein k is 0, 1 or 2; or R^{16u} is selected from 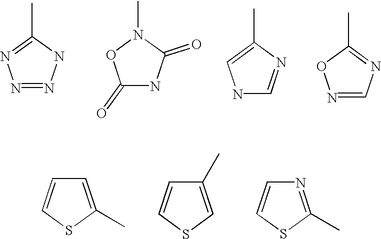
or a pharmaceutically acceptable salt thereof.

17. The use according to anyone of the claims 1-3 and 16 wherein the compound is selected from the following: 1-(3-(10,11-Dihydro-5H-dibenzo[b,f]azepin-5-yl)-(2R)-methyl-1-propyl)-(3R)- piperidinecarboxylic acid; 1-(3-(10,11-Dihydro-5H-dibenzo[b,f]azepin-5-yl)-(2R)-methyl-1-propyl)-4- piperidinecarboxylic acid; 1-(3-(10,11-Dihydro-5H-dibenzo[b,f]azepin-5-yl)-(2R)-methyl-1-propyl)-(2R)- piperidinecarboxylic acid; 1-(4-(1 ,11-Dihydro-5H-dibenzo[b,azepin-5-yl)-(2Z)-butenyl)-(3R)-piperid inecarboxylic acid; 1-(3-(10,11-Dihydro-5H-dibenzo[a,d]cyclohepten-5-ylidene)-1-propionyl)-(3R)- piperidine-carboxylic acid; 1-(2-(10,11-Dihydro-5H-dibenzo[a,d]cyclohepten-5-yl)-1-ethyl)-(3R)-piperidine- carboxylic acid; 1-(4-(10,11-Dihydro-5H-dibenzo[b,f]azepin-5-yi)-(2E)-butenyl)-(3R)-piperidinecarboxylic acid; 1-(2-(I 0,11-Dihydro-5H-dibenzo[b,f]azepin-5-yl)-1-methyl-1-ethyl)-(3R)- piperidinecarboxylic acid; 1-(3-(l 0,11-Dihydro-5H-dibenzo[b,f]azepin-5-yl)-2-methyl-3-oxopropyl)-(3R)- piperidinecarboxylic acid; acid; 1-(3-(10,11-Dihydro-5H-dibenzo[b,f]azepin-5-yl)-1-methyl-I-propyl)-(3R)- piperidinecarboxylic acid; 1-(3-(l 0,11-Dihydro-5H-dibenzo[b,f]azepin-5-yl)-2-hydroxy-1-propyl)-(3R)- piperidinecarboxylic acid; 1-(2-(10,11-Dihydro-dibenzo[b,f]azepin-5-ylmethyl)-1-pentyl)-(3R)-piperidinecarboxylic acid; 1-(3-(3-Chloro-10,11-dihydro-5H-dibenzo[b,f]azepin-5-yl)-(2R)-methyl-1-propyl)-(3R)- piperidinecarboxylic acid; 11-(3-(3-Trifluoromethy l- 0,11-dihydro-5H-dibenzo[b,f]azepin-5-yl)-(2R)-methyl-1- propyl)-(3R)-piperidinecarboxylic acid; 1-(3-(3-Methyl-10,11-dihydro-5H-dibenzo[b,f]azepin-5-yl)-(2R)-methyl-1-propyl)-(3R)- piperidinecarboxylic acid; 1-(3-(3-Methoxy-10,11-dihydro-5H-dibenzo[b,f]azepin-5-yl)-(2R)-methyl-1-propyl)-(3R)- piperidinecarboxylic acid; 1-(3-(2-Chloro-10,11-dihydro-5H-dibenzo[b,f]azepin-5-yl)-(2R)-methyl-1-propyl)-(3R)- piperidinecarboxylic acid; 2-(4-(3-(10,11-Dihydro-5H-dibenzo[b,f]azepin-5-yl)-(2R)-methyl-1-propyl)-1- piperazinyl)-nicotinic acid; 1-(2-(10,11-Dihydro-5H-dibenzo[a,dlcyclohepten-5-ylidene)-1-propyl)-(3R)- piperidinecarboxylic acid; 1-(2-(10,1 -Dihydro-5H-dibenzo[b,f]azepin-5-yl)-cyclopropylmethyl)-(3R)- piperidinecarboxylic acid; 1-(2-(10,11-Dihydro-5H-dibenzo[b,f]azepin-5-yl)-cyclopentylmethyl)-(3R)- piperidinecarboxylic acid; 1-(2-(10,11-Dihydro-5H-dibenzo[a,d]cyclohepten-5-yl)-1-ethyl)-(3R)- piperidinecarboxylic acid; (R)-1-(3-(10,11-Dihydro-5H-dibenzo[b,f]azepin-5-yl)-3-oxopropyl)-3-piperidinecarboxylic acid; (R)-1-(4-(10 ,11-Dihydro-5H-dibenzo[a,d]cyclohepten-5-yl)-benzyl)-3-piperidinecarboxylic acid; (R)-1-(4-(10,11-Dihydro-5H-dibenzo[b,qazepin-5-yl)-2-butyn-1-yi)-3-piperidinecarboxylic acid (R)-1-((2R)-Methyl-3-(3-methyl-10,11-dihydro-5H-dibenzo[b,f]azepin-5-yi)-1-propyl)-4- piperidinecarboxylic acid; (R)-1-(3-(10,11-Dihydro-5H-dibenzo[b,f]azepin-5-yl)1-methylpropyl)-3-piperidinecarboxylic acid; (R)-1-(2-(10,11-dihydro-5H-dibenzo[b ,flazepin-5-yl)-1-methyl-ethyl)-3-piperidinecarboxylic acid; (R)-1-(2-(10,11-Dihydro-5H-dibenzo[a,d]cyclohepten-5-ylidene)-1-propyl)-3-piperidine- carboxylic acid; (R)-1-(1,11-Dihydro-5H-dibenzo[a,d]cyclohepten-5-yl)methyl)-3-piperidinecarboxylic acid; 1-(3-(10,11-Dihydro-5H-dibenzo[b,f]azepin-5-yi)-(2R)-methyl-1-propyl)- 3-pyrrolidinylacetic acid; 2-(1-(3-(10,11-Dihydrodibenzo[b,f]azepin-5-yl)-(2R)-methylpropyl)-4-piperazinyl)-nicotinic acid; (R)-1-(2-(10,11-Dihydro-5H-dibenzo[b,f]azepin-5-ylmethyl)-1-pentyl)-3-piperidinecarboxylic acid; 2-(4-(3-(10,11-Dihydro-5H-dibenzo[b,f]azepin-5-yl)-2-hydroxypropyl)piperazin-1-yl)nicotinic acid; 1-(3-(10,11-Dihydro-5H-dibenzo[b,f]azepin-5-y)-2-methyl-3-oxo-propyl)-3-piperidinearboxylic acid; (R)-1-(3-(10,11-Dihyd ro-5H-dibenzo[b,f]azepin-5-yi)-1-propionyl)-3-piperidinecarboxylic acid; 1-(3-(10,11-Dihydro-5H-dibenzo[b,f]azepin-5-yl)-1-propionyl)-4-piperidinecarboxylic acid; (R)-1-(2-(10,11-Dihydro-5H-dibenzo[b,f]azepin-5-ylcarbonyl)-1-benzyl)-3-piperidinecarboxylic acid; (R)-1-(2-(10,11-Dihydro-5H-dibenzo[b,f]azepin-5-ylmethyl)-benzyl)-3-piperidinecarboxylic acid; (R)-1-(3-(10,11-Dihydro-5H-dibenzo[a,d]cyclohepten-5-yl)-3-oxo-1-propyl)-3- piperidinecarboxylic acid; 1-(3-(3-Chloro-10,11-dihydro-5H-dibenzo[b,f]azepin-5-yl)-(2R)-methylpropyl)-4-piperidine- carboxylic acid; 1-(3-(10,11-Dihydro-5H-dibenzo[b ,f]azepin-5-yl)-2-hydroxy-propyl)-4-piperidinecarboxylic acid; (R)-1-(3-(10,11-Dihydro-5H-dibenzo[b,f]azepin-5-yl)-2-hydroxypropyl)-3-piperidinecarboxylic acid; 1-(3-(10,11-Dihydro-5H-dibenzo[b,f]azepin-5-yl)-2-propoxypropyl)-4-piperidinecarboxylic acid; (R)-1-(2-(N-(10,11-dihydro-5H-dibenzo[a,d]cyclohepten-5-yl)-N-methylamino)ethyl)- 3-piperidinecarboxylic acid,
or a pharmaceutically acceptable salt thereof.

18. The use according to anyone of the claims 1-3 wherein in formula Ia R¹, R^{1a}, R² and R^{2a} independently are hydrogen, halogen, trifluoromethyl, hydroxy, C -alkyl, .C₁₋₆-alkoxy or methylthio, —NR⁷R3 or —SO₂NR⁷R⁸ wherein R⁷ and R⁸ independently are hydrogen or C₁₋₆-alkyl; and Y is >CH—O— or >CH—S(O)_{y} wherein y is 0, 1 or 2, or —N(R⁸)— wherein R⁸ is hydrogen or C₁₋₆- alkyl; and X is completion of an optional bond, ortho-phenylene, —O—, —S—, —C(R⁷R3)—, —CH₂CH₂—, —CH═CH- CH₂—, —CH₂—CH═CH—, —CH₂—(C═O)—, —(C═O)—CH₂—, —CH₂CH₂CH₂—, —CH═CH—, —N(R⁸)—(C═O)—, - (C═O)—N(R⁸)—, —O—CH₂—, —CH₂—O—, —OCH₂0—, —CH₂0CH₂—, —S—CH₂—, —CH₂—S—, —(CH₂)N(R⁸)—, - N(R⁸)(CH₂)—, —N(CH₃)SO₂—, -S0₂N(CH₃)—, —CH(R⁹)CH₂—, —CH₂CH(R⁹)—, —(C═O)—, —N(R⁸)— or - (S═O)— wherein R⁷ and R⁸ independently are hydrogen or C₁l-alkyl; and wherein R⁹ is Cl-6 alkyl or phenyl; and p and q independently are 0 or 1; and r is 1, 2, 3 or 4; and Z is selected from [chemical structure]
wherein g is 0, 1 or 2; and
R^{11u} is hydrogen, C₁₋₆-alkyl, C₁₋₆-alkoxy or phenyl optionally substituted with halogen, trifluoromethyl, hydroxy, C₁₋₆-alkyl or C₁₋₆-alkoxy; and
R^{12u} is —(CH₂)ₕOH or (CH₂)jCoRi⁷u wherein h is 0, 1, 2, 3, 4, 5 or 6 and j is 0 or 1 and wherein R^{17u}is —OH, NH R²⁰u or C₁4-alkoxy wherein R^{20u}is hydrogen or C₁₋₆-alkyl; and
R^{13u} is hydrogen, halogen, trifluoromethyl, hydroxy, C, e-alkyJ or C₁₋₆-alkoxy; and
R^{14u} is hydrogen or C₁₋₆-alkyl; and
C is C₁₋₆-alkylene, C₂6-alkenylene or C₂₆-alkynylene; and [symbol]
is optionally a single bond or a double bond; and
R^{18u} is selected from 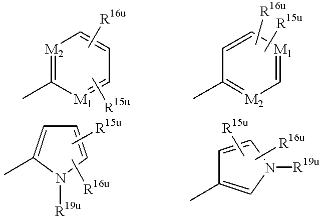
wherein M₁ and M₂ independently are C or N; and R^{19u}is hydrogen, C₁₋₆-alkyl, phenyl or benzyl; and R^{15u}is hydrogen, halogen, trifluoromethyl, nitro or cyano; and R^{16u}is hydrogen, halogen, trifluoromethyl, nitro, cyano, —(CH₂)kCORl⁷U, —(CH₂)kQH or- (CH₂)kSo₂Rl⁷u wherein k is 0, 1 or 2; or
R^{16u} is selected from 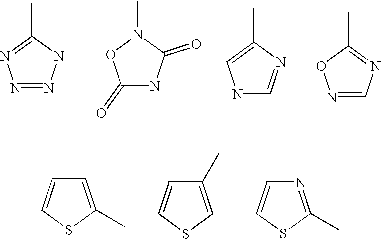
or a pharmaceutically acceptable salt thereof.

19. The use according to anyone of the claims 1-3 and 18 wherein the compound is selected from the following: 1-(2-(10,11-Dihydrodibenzo[b,f]thiepin-10-yloxy)-1-ethyl)-(3R)-piperidinecarboxylic acid; 1-(2-(2-Chloro-10,11-dihydrodibenzo[b,f]thiepin-10-yloxy)-1-ethyl)-3- piperidinecarboxylic acid; 1-(2-(2-Chloro-10,11-dihydrodibenzo[b,f]thiepin-10-yloxy)-1-ethyl)-4- piperidinecarboxylic acid; 1-(2-(2-Methyl-10,11-dihydrodibenzo[b,f]thiepin-10-yloxy)-1-ethyl)-4- piperidinecarboxylic acid; 1-(2-(2-Methyl- 10,11-d ihyd rodibenzo[b,f]thiepin-10-yloxy)-1-ethyl)-3- piperidinecarboxylic acid; 1-(2-(8-Chloro-10,11-dihydrodibenzo[b,f]thiepin-10-yloxy)-1-ethyl)-3- piperidinecarboxylic acid; 1-(2-(8-Methylthio-10,11-dihydrodibenzo[b,f]thiepin-10-yloxy)-1-ethyl)-3- piperidinecarboxylic acid; (R)-1-(2-(10,11-Dihydrodibenzo[b,floxepin-10-yloxy)ethyl)-3-piperidinecarboxylic acid; (R)-1-(2-(2-Chloro-10,11-dihydrodibenzo[b,f]thiepin-10-ylsulfanyl)ethyl)-3- piperidinecarboxylic acid; (R)-1-(11H-Dibenz[b,f][1 ,4]oxathiepin-11-ylmethyl)-3-piperidinecarboxylic acid; (R)-1-(2-(2-Chloro-7-fluoro-10,11-dihydrodibenzo[b,f]thiepin-10-yloxy)ethyl)-3- piperidinecarboxylic acid; (R)-1-(2-(2,4-Dichloro-10,11-dihydrodibenzo[b,f]thiepin-10-yloxy)ethyl)-3-piperidinecarboxylic acid,
or a pharmaceutically acceptable salt thereof.

20. The use according to anyone of the claims 1-3 wherein in formula Ia R¹, R¹a, R² and R^{2a} independently are hydrogen, halogen, trifluoromethyl, hydroxy, Cl-6alkyl or CI-alkoxy; and Y is >N—CH₂—, >CH—CH₂— or >C═CH— wherein only the underscored atom participates in the ring system; and X is ortho-phenylene, —O—, —S—, —C(R⁷R⁸)—, —CH₂CH₂—, —CH═CH—CH₂—, —CH₂—CH═CH—, —CH₂- (C═O)—, —(C═O)—CH₂—, —CH₂CH₂CH₂—, —CH═CH—, —N(R⁸)—(C═O)—, —(C═O)—N(R⁸)—, —O—CH₂—, —CH₂- O—, —OCH₂0—, —S—CH₂—, —CH₂—S—, —(CH₂)N(R⁸)—, —N(R)(CH₂)—, —N(CH₃)SO₂—, —SO₂N(CH₃)—, - CH(R⁹)CH₂—, —CH₂CH(R⁹)—, —(C═O)—, —N(R⁸)— or —(S═O)— wherein R⁷ and R⁸ independently are hydrogen or C₁₋₆-alkyl; and wherein R⁹ is C₁-6alkyl or phenyl; and p and q are 0; and r is 1, 2or3; and Z is selected from 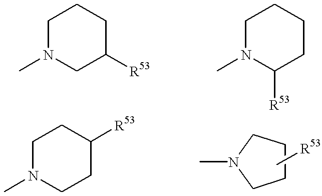
wherein R⁵³ is —(CH₂)ppCOOH wherein pp is 2, 3, 4, 5 or 6; or
a pharmaceutically acceptable salt thereof.

21. The use according to anyone of the claims 1-3 and 20 wherein the compound is selected from the following: 3-(1-(3-(10,11-Dihydrodibenzo[a,d]cyclohepten-5-ylidene)-1-propyl)piperidin-3-yl)propionic .acid; 3-(1-(3-(10,11-Dihydrodibenzo[b,f]azepin-5-yi)-1-propyl)piperidin-3-yl)propionic acid; 3-(1-(2-(10,11-Dihydrodibenzo[ad]cyclohepten-5-ylidene)ethyl)piperidin-4-yl)propionic acid; 3-(1-(3-(10,11-Dihydro-5H-dibenzo[a,d]cyclohepten-5-ylidene)-1-propyl)piperidin-4- yl)propionic acid; 3-(1-(3-(10,11-Dihydro-5H-dibenzo[b,f]azepin-5-yl)-1-propyl)piperidin-4-yl)propionic acid; 3-(1-(3-(Thioxanthen-9-ylidene)-1-propyl)piperidin-4-yl)propionic acid; ³-(l-(³-(Xanthen-9-ylidene)-1-propyl)piperidin-4-yl)propionic acid; 3-(1-(3-(12H-Dibenzo[d,g][1 ,3]dioxocin-12-ylidene)-1-propyl)piperidin-4-yl)propionic acid; 4-(1-(3-(10,11-Dihydro-5H-dibenzo[a,d]cyclohepten-5-ylidene)-1-propyl)piperidin-4-yl)-butyric acid; 3-(1-(3-(10,11-Dihydro-5H-dibenzo[a,d]cyclohepten-5-ylidene)-1-propyl)piperidin-2-yl)- propionic acid; 3-(1-(3-(1-Bromo-10,11-dihydro-5H-dibenzo[a,d]cyclohepten-5-ylidene)-1-propyl)piperidin-4- yl)propionic acid; 3-(1-(3-(2-Fluoro-10,11-dihydro-5H-dibenzo[a,d]cyclohepten-5-ylidene)-1-propyl)piperidin-4- yl)propionic acid; 3-(1-(3-(2-Trifluoromethyl-10,11-dihydro-5H-dibenzo[a,d]cyclohepten-5-ylidene)-1-propyl)- piperidin-4-yl)propionic acid; 3-(1-(3-(2-Hydroxy-10,11-dihydro-5H-dibenzo[a,d]cyclohepten-5-ylidene)-1-propyl)piperidin- 4-yl)propionic acid; 3-(l-(3-(2-Methyl-10,11-dihydro-5H-dibenzo[a,d]cyclohepten-5-ylidene)-1-propyl)piperidin-4- yl)propionic acid; 3-(1-(3-(2-Methoxy-10,11-dihydro-5H-dibenzo[a,d]cyclohepten-5-ylidene)-1-propyl)-piperidin- 4-yl)propionic acid; 3-(1-(3-(10,11-Dihydro-5H-dibenzo[a,d]cyclohepten-5-yl)-1-propyl)piperidin-4-yl)propionic acid; 3-(1-(3-(6,11-Dihydro-dibenz[b,e]thiepin-11-ylidene)-1-propyl)piperidin-4-yl)propionic acid; 3-(1-(3-(2-Fluoro-6,11-dihydro-dibenz[b,e]thiepin-11-ylidene)-1-propyl)piperidin-4-yl)- propionic acid; 4-(1-(3-(6,11-Dihydro-dibenz[b,e]thiepin-11-ylidene)-1-propyl)piperidin-4-yl)butyric acid; 3-(1-(3-(6,11-Dihydro-dibenz[b,e]thiepin-11-ylidene)-1-propyl)piperidin-3-yl)propionic acid; 3-(1-(3-(6,11-Dihydro-dibenz[b,e]thiepin-11-ylidene)-1-propyl)piperidin-2-yl)propionic acid; 3-(1-(3-(10,11-Dihydro-5H-dibenzo[a,d]cyclohepten-5-ylidene)-1-propyl)pyrrolidin-3-yl)- propionic acid; 4-(1-(3-(10,11-Dihydro-5H-dibenzo[a,d]cyclohepten-5-ylidene)-1-propyl)pyrrolidin-3-yl)- butyric acid; 3-(1-(3-(6,11-Dihydro-dibenz[b,e]thiepin-11-ylidene)-1-propyl)pyrrolidin-3-yl)propionic acid; 3-(1-(3-(1 OH-Anthracen-9-ylidene)-1-propyl)pyrrolidin-3-yl)propionic acid; 3-(1-(3-(Dibenzo[a,d]cyclohepten-5-ylidene)-1-propyl)pyrrolidin-3-yl)propionic acid; 3-(1-(3-(1 OH-Anthracen-9-ylidene)-1-propyl)piperidin-4-yl)propionic acid; 3-(1-(3-(Dibenzo[a,d]cyclohepten-5-ylidene)-1-propyl)piperidin-4-yl)propionic acid; 5-(1-(3-(10,11-Dihydro-5H-dibenzo[a,d]cyclohepten-5-yl)-1-propyl)piperidin-4-yl)pentanoic acid; 5-(1-(3-(6,11-Dihydro-dibenz[b,e]thiepin-11-ylidene)-1-propyl)piperidin-4-yl)pentanoic acid; 5-(1-(3-(Thioxanthen-9-ylidene)-1-propyl)piperidin-4-yl)pentanoic acid; 5-(1-(3-(12H-Dibenzo[d,g][1 ,3]dioxocin-12-ylidene)-1-propyl)piperidin-4-yl)pentanoic acid, or a pharmaceutically acceptable salt thereof.

22. The use according to anyone of the claims 1-3 wherein in formula Ia R¹, R^{1a}, R² and R^{2a} independently are hydrogen, halogen, trifluoromethyl, hydroxy, C₁₋₆-alkyl or C₁₋₆-alkoxy; and Y is >N—CH₂—, >CH—CH₂—, >C═CH- or >CH—O- wherein only the underscored atom participates in the ring system; and X is ortho-phenylene, —O—, —S—, —C(R⁷R⁸)—, —CH₂CH₂—, —CH═CH—CH₂—, —CH₂—CH═CH—, —CH₂- (C═O)—, —(C═O)—CH₂—, —CH₂CH₂CH₂—, —CH═CH—, —N(R⁸)—(C═O)—, —(C═O)—N(R⁸)—, —O—CH₂—, —CH₂- O—, —OCH₂0—, —S—CH₂—, —CH₂—S—, —(CH₂)N(R⁸)—, —N(R⁸)(CH₂)—, —N(CH₃)SO₂—, -So₂N(CH₃)—, - CH(R⁹)CH₂—, —CH₂CH(R⁹)—, —(C═O)—, —N(R⁸)— or —(S═O)— wherein R⁷ and R⁸ independently are hydrogen or C₁6-alkyl; and wherein R⁹ is C₁₋₆-alkyl or phenyl; and p and q are 0; and r is 1, 2or3; and Z is 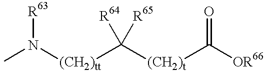
wherein tt and t independently are 0, 1 or 2; and R⁶³ is H, C₁₋₆-alkyl or optionally substituted benzyl; R⁶⁴ and R⁶⁵ independently are H, Cl₈-alkyl, C3-₇-cycloalkyl, phenyl, thienyl, benzyl, or R⁶⁴ and R⁶⁵ together with the C-atom they are attached to form a 3 - 8 membered carbocyclic ring; and R⁶⁶ is H or C₁₋₆-alkyl; or a pharmaceutically acceptable salt thereof.

23. The use according to anyone of the claims 1-3 and 22 wherein the compound is selected from the following: 1-(2-(10,11-Dihydrodibenzo[b ,fth iepin-10-yloxy)-1-ethyl)-(3R)-piperidinecarboxylic acid; 1-(2-(2-Chloro-10,11-dihydrodibenzo[b,f]thiepin-10-yloxy)-1-ethyl)-3- piperidinecarboxylic acid; 1-(2-(2-Chloro-10,11-dihydrodibenzo[b,f]thiepin-10-yloxy)-1-ethyl)-4- piperidinecarboxylic acid; 1-(2-(2-Methyl-10,11-dihydrodibenzo[b,f]thiepin-10-yloxy)-1-ethyl)-4- piperidinecarboxylic acid; 1-(2-(2-Methyl-10,11-dihydrodibenzo[b,f]thiepin-10-yloxy)-1-ethyl)-3- piperidinecarboxylic acid; 1-(2-(8-Chloro-10,11-dihydrodibenzo[b,f]thiepin-10-yloxy)-1-ethyl)-3- piperidinecarboxylic acid; 1-(2-(8-Methylthio-10,11-dihydrodibenzo[b ,f]thiepin- 10-yloxy)-1-ethyl)-3- piperidinecarboxylic acid; (R)-1-(2-(10,11-Dihydrodibenzo[b,floxepin-10-yloxy)ethyl)-3-piperidinecarboxylic acid; (R)-1-(2-(2-Chloro-10,11-dihydrodibenzo[b,f]thiepin-10-ylsulfanyl)ethyl)-3- piperidinecarboxylic acid; (R)-1-(11H-Dibenz[b,fl[1 ,4]oxathiepin-11-ylmethyl)-3-piperidinecarboxylic acid; (R)-1-(2-(2-Chloro-7-fluoro-10,11-dihydrodibenzo[b,f]thiepin-10-yloxy)ethyl)-3- piperidinecarboxylic acid; (R)-1-(2-(2,4-Dichloro-10,11-dihydrodibenzo[b,f]th iepin-10-yloxy)ethyl)-3-piperidinecarboxylic acid, or a pharmaceutically acceptable salt thereof.

24. The use according to anyone of the claims 1-3 wherein in formula Ia R¹, R¹a, R² and R^{2a} independently are hydrogen, halogen, trifluoromethyl, hydroxy, C₁₋₆-alkyl or Cl-6alkoxy; and Y is >N—CH₂—, >CH—CH₂— or >C═CH— wherein only the underscored atom participates in the ring system; and X is ortho-phenylene, —O—, —S—, —C(R⁷R3)—, —CH₂CH₂—, —CH═CH—CH₂—, —CH₂—CH═CH—, —CH₂- (C═O)—, —(C═O)—CH₂—, —CH₂CH₂CH₂—, —CH═CH—, —N(R⁸)—(C═O)—, —(C═O)—N(R⁸)—, —O—CH₂—, —CH₂- O—, —OCH₂0—, —S—CH₂—, —CH₂—S—, —(CH₂)N(R⁸)—, —N(R⁸)(CH₂)—, —N(CH₃)SO₂—, —SO₂N(CH₃)—, - CH(R⁹)CH₂—, —CH₂CH(R⁹)—, —(C═O)—, —N(R⁸)— or —(S═O)— wherein R⁷ and R3 independently are hydrogen or C₁-6alkyl; and wherein R⁹ is C₁-6alkyl or phenyl; and p and q are 0; and r is 0, 1 or 2; and Z is selected from 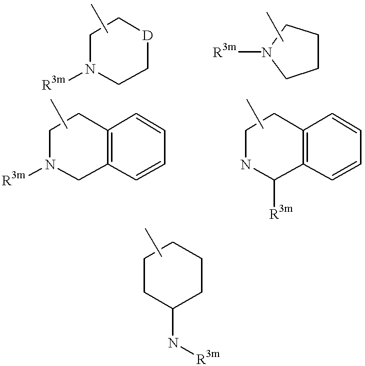
wherein D is —CH₂—, —O—, —S— or —N(R⁷)— wherein R⁷ is H or C₁₋₆-alkyl; and R^{3m} is —(CH₂)mmOH or —(CH₂)mpCOR⁴ wherein mm and mp are 1, 2, 3 or 4 and R⁴ is OH, NH₂, NHOH or C₁8-alkoxy; or a pharmaceutically acceptable salt thereof.

25. The use according to anyone of the claims 1-3 and 24 wherein the compound is selected from the following: 3-(3-(10,11-Dihydro-5H-dibenzo[b,f]azepin-5-ylmethyl)-pyrrolid in-1-yl)-propionic acid; (2-(2-(10,11-Dihydro-5H-dibenzo[b,f]azepin-5-ylmethyl)-morpholin-4-yl)-acetic acid; (3-(10,11-Dihydro-5H-dibenz[(b,flazepin-5-ylmethyl)-1-piperidyl)acetic acid, or a pharmaceutically acceptable salt thereof.

26. The use according to anyone of the claims 1-3 wherein in formula Ia R¹, R^{1a}, R² and R^{2a} independently are hydrogen, halogen, cyano, trifluoromethyl, methylthio, hydroxy, C₁₋₆-alkyl or C₁₋₆-alkoxy; and Y is >N—, >CH—, >N—(C═O)— or >C═C(R⁸)—, wherein only the underscored atom participates in the ring system and R⁸ is hydrogen or C₁₋₆-alkyl; and X is ortho-phenylene, —O—, —S—, —C(R⁷R⁸)—, —CH₂CH₂—, —CH═CH—CH₂—, —CH₂—CH═CH—, —CH₂- (C═O)—, —(C═O)—CH₂—, —CH₂CH₂CH₂—, —CH═CH—, —N(R⁸)—(C═O)—, —(C═O)—N(R⁸)—, —O—CH₂—, —CH₂- O—, —OCH₂0—, —CH₂0CH₂—, —S—CH₂—, —CH₂—S—, —(CH₂)N(R⁸)—, —N(R⁸)(CH₂)—, —N(CH₃)SO₂—, - SO₂N(CH₃)—, —CH(R)CH₂—, —CH₂CH(R⁹)—, —(C═O)—, —N(R⁸)— or —(S═O)— wherein R⁷ and R⁸ independently are hydrogen or C₁₋₆-alkyl; and wherein R⁹ is C₁₋₆-alkyl or phenyl; and p and q are 0; and r is O, 1,2,3or4; and Z is 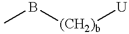
wherein b is 0, 1, 2, 3 or 4; and B is —CH═CR⁴⁹—, —CR⁴⁹═CH—, —C═C—, —(C═O)—, —(C═CH₂)—, —(CR⁴⁹R⁴⁰)—, —CH(OR⁴¹)—, - CH(NHR⁴¹)—, phenylene, C₃₋₇-cycloalkylene or the completion of a bond, wherein R⁴⁹ and R⁴⁰ independently are hydrogen, C₁₋₆-unbranched alkyl, C₃₋₆-branched alkyl or C₃₋₇-cycloalkyl and wherein R41 is hydrogen or C₁₋₆alkyl; and U is 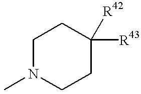
wherein R⁴² is hydrogen, —(CH₂)cOH or —(CH₂)dCOR⁴⁷ wherein c is 0, 1, 2, 3, 4, 5 or 6 and d is 0 or I and wherein R⁴⁷ is —OH, —NHR⁴⁴ or C₁₋₆-alkoxy wherein R⁴⁴ is hydrogen or C₁₋₆-alky; and R is cyano, —NR⁴⁵R⁴⁶, —NR⁴⁵—V or —(CHR⁴⁸)e—V wherein R⁴⁵ and R⁴⁶ independently are hydrogen or C₁₋₆-alkyl and wherein e is 0, 1, 2, 3, 4, 5 or 6 and wherein R⁴⁸ is hydrogen, halogen, cyano, trifluoromethyl, hydroxy, Cl-6alkyl, C₁₋₆-alkoxy, —NR⁴⁵R⁴5 or —COOH, and wherein V is C₃₈-cycloalkyl, aryl or heteroaryl, which rings may optionally be substituted with one or more halogen, cyano, trifluoromethyl, hydroxy, methylthio, C₁₋₆-alkyl or C₁₋₆-alkoxy; or a pharmaceutically acceptable salt thereof.

27. The use according to anyone of the claims 1-3 and 26 wherein the compound is selected from the following: 1-(3-(10,11-Dihydro-5H-dibenzo[a, d]cyclohepten-5-ylidene)-1-propyl)-4-phenyl-4- piperidinecarboxylic acid; 4-(4-Chlorophenyl)-1-(3-(10,11-dihydro-5H-dibenzo[a,d]cyclohepten-5-ylidene)-1-propyl)-4- piperidinecarboxylic acid; 4-(4-Methylphenyl)-1-(3-(10,11-dihydro-5H-dibenzo[a,d]cyclohepten-5-ylidene)-1-propyl)-4- piperidinecarboxylic acid; 1-(3-(10,11-Dihydro-5H-dibenzo[a,d]cyclohepten-5-ylidene)-1-propyl)-4-anilino-4- piperidinecarboxamide; 2-(1-(3-(10,11-Dihydro-5H-dibenzo[a,d]cyclohepten-5-ylidene)-1-propyl)-4-piperidyl)-2- phenylacetonitrile; 2-(1-(3-(10,11-Dihydro-5H-dibenzo[a,d]cyclohepten-5-ylidene)-1-propyl)-4-piperidinyl)-2- phenylacetic acid; 1-(3-(10,11-Dihydro-5H-dibenzo[a,d]cyclohepten-5-ylidene)-1-propyl)-4-cyano-4 piperidinecarboxylic acid, or a pharmaceutically acceptable salt thereof.

28. The use according to anyone of the claims 1-3 wherein in formula Ib R^{1b} and R^{2b} independently are hydrogen, halogen, trifluoromethyl, hydroxy, C₁₋₆-alkyl or Cl- alkoxy; and R^{3b} is hydrogen or C₁₋₆-alkyl; and Ab is CI₃-alkylene; and Yb is >CH—CH₂—, >C═CH—, >CH—O—, >C═N—, >N—CH₂— wherein only the underscored atom participates in the ring system; and Zb is selected from [chemical structure]
wherein nb is 1 or 2; and Rlb is hydrogen or Cl-6alkyl; and Rl²b is hydrogen, C₁₋₆-alkyl, Cl-6alkoxy or phenyl optionally substituted with halogen, trifluoro- methyl, hydroxy, C₁I-6alkyl or C₁₋₆-alkoxy; and R1³b is hydrogen, halogen, trifluoromethyl, hydroxy, C₁₋₆-alkyl or C₁I-alkoxy; and R1⁴b is —(CH₂)mbOH or —(CH₂)tbCOR5b wherein mb is 0, 1, 2, 3, 4, 5 or 6 and tb is 0 or 1 and wherein R⁵b is —OH, NH₂, —NHOH or C₁₋₆-alkoxy; and R⁶b is C₁alkyl or BbCORl⁵b, wherein Bb is C,-alkylene, C₂6-alkenylene or C₂6-alkynylene and R⁵b is the same as above; and
I is optionally a single bond or a double bond; or a pharmaceutically acceptable salt thereof.

29. The use according to anyone of the claims 1-3 and 28 wherein the compound is selected from the following:
1-(3-(12H-Dibenzo[d,g][1 ,3]dioxocin-12-ylidene)-1-propyl)-3-piperidinecarboxylic acid;
(R)-1-(3-(12H-Dibenzo[d ,g] [1 ,3]dioxocin-12-ylidene)-1-propyl)-3-piperidinecarboxylic acid;
(R)-1-(3-(12H-Dibenzo[d,g][1 ,3]dioxocin-12-ylidene)-1-propyl)-3-piperidinecarboxylic acid ethyl ester;
1-(3-(12H-Dibenzo[d,g][1 ,3]dioxocin-12-ylidene)-1-propyl)-4-piperidinecarboxylic acid;
(R)-1-(3-(2, 10-Dichloro-12H-dibenzo[d,g][1 ,3]dioxocin-12-ylidene)-1-propyl)-3- piperidinecarboxylic acid;
,-(3-(12H-Dibenzo[d,g][1,3]dioxocin-12-ylidene)-1-propyl)-3-pyrrolidineacetic acid;
1-(3-(2,1 O-Dichloro-12H-dibenzo [d,g[1 ,3]dioxocin-12-ylidene )-1-propyl)-3-pyrrolidineacetic acid;
(R)-1-(2-(12H-Dibenzo[d,g][1 ,3]dioxocin-12-yloxy)-1-ethyl)-3-piperidinecarboxylic acid; (R)-1-(2-(2, 10-Dichloro-12H-dibenzo[d,g][1 ,3]dioxocin-12-yloxy)-1-ethyl)-3- piperidinecarboxylic acid; (R)-1-(3-(2-Chloro-12H-dibenzo[d,g][1 ,3,6]dioxazocin-12-yl)-1-propyl)-3-piperidinecarboxylic acid;
I-(3-(12H-Dibenzo[d,g][1 ,3,6]dioxazocin-12-yl)-1-propyl)-4-piperidinecarboxylic acid; 2-Chloro-12-(3-dimethylamino)propylidene-12H-dibenzo[d,g][1 ,3]dioxocine; 2,10-Dichloro-12-(2-dimethylamino)ethoxy-12H-dibenzo[d,g][1 ,3]dioxocine; 2,10-Dichloro-12-(3-dimethylamino)propyl-12H-dibenzo[d,g][1 ,3]dioxocine; 2,10-Dichloro-12-(3-dimethylamino-1-methyl)ethoxy-12H-dibenzo[d,g][1 ,3]dioxocine; 3-Chloro-12-(2-dimethylaminopropylidene)-12H-dibenzo[d,g][1,3]dioxocine; 3-Chloro-12-(3-dimethylamino)propylidene-12H-dibenzo[d,g][1,3]dioxocine; 3-Chloro-12-(3-dimethylamino-1-methylpropylidene)-12H-dibenzo-[d,g][1 ,3]dioxocine; 2-Fluoro-12-(3-dimethylamino)propylidene-12H-dibenzo[d,g][1 ,3]dioxocine; 2-Methyl-12-(3-(4-methyl-I -piperazinyl)propylidene)-12H-dibenzo[d, g][1 ,3]dioxocine; 2-Chloro-12-(3-(4-methyl-1-piperazinyl)propylidene)-12H-dibenzo[d,g][1 ,3]dioxocine; 3-Chloro-I 2-(3-(4-methyl-I -piperazinyl)propylidene)-12H-dibenzo[d,g][1,3]dioxocine; 1-(3-(12H-Dibenzo[d,g][1,3]dioxocin-12-ylidene)propyl)-3-piperidinecarboxylic acid ethyl ester; 1-(3-(12H-Dibenzo[d,g][1 ,3]dioxocin-12-ylidene)propyl)-3-piperidinecarboxylic acid, or a pharmaceutically acceptable salt thereof.

30. The use according to anyone of the claims 1-3 wherein in formula Ic R^{1c} and R^{2c} independently are hydrogen, halogen, trifluoromethyl, hydroxy, C₁₋₆-alkyl or C₁₋₆-alkoxy; and X_{c} s ortho-phenylene, —O—, —S—, —C(R^{6c}R^{7c}), —CH₂CH₂—, —CH═CH—CH₂—, —CH₂—CH═CH—, —CH₂-(C═O)—, —(C═O)—CH₂—, —CH₂CH₂CH₂—, —CH═CH—, —N(R^{8c})—(C═O)—, —(C═O)—N(R^{8c})—, —O—CH₂—, —CH₂-O—, —OCH₂O—, —S—CH₂—, —CH₂—S—, —(CH₂)N(R^{8c})—, —N(R^{8c})(CH₂)—, —N(CH₃)SO₂—, —SO₂N(CH₃)—, -CH(R^{10c})CH₂—, —CH₂CH(R^{10c})—, —(C═O)—, —N(R^{9c})— or —(S═O)— wherein R^{6c}, R^{7c}, R^{8c} and R^{9c} independently are hydrogen or C₁₋₆-alkyl, and wherein R^{10c} is C₁₋₆-alkyl or phenyl; and Y_{c} is C or N; and II,, is optionally a single bond or a double bond, and [symbol] is a single bond when Yc is N; and mc is 1,2,3,4, 5 or 6; and Z_{c} is —COOR^{3c} or 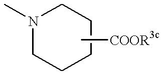
wherein R^{3c} is H or C₁₋₆-alkyl; and a pharmaceutically acceptable salt thereof.

31. The use according to anyone of the claims 1-3 and 30 wherein the compound is selected from the following: 1-(2-(10,11-Dihydrodibenzo[b,f]thiepin-10-yloxy)-1-ethyl)-(3R)-piperidinecarboxylic acid; 1-(2-(2-Chloro-10,11-dihydrodibenzo[b,f]thiepin-10-yloxy)-1-ethyl)-3-piperidine- carboxylic acid; 1-(2-(2-Chloro- 10,11-dihydrodibenzo[b,f]thiepin-10-yloxy)-1-ethyl)-4-piperidine- carboxylic acid; 1-(2-(2-Methyl-10,11-dihydrodibenzo[b,f]thiepin-10-yloxy)-1-ethyl)-4-piperidine- carboxylic acid; I -(2-(2-Methyl-10,11-dihydrodibenzo[b,f]thiepin-10-yloxy)-1-ethyl)-3-piperidine- carboxylic acid; 1-(2-(8-Chloro-10,11-dihydrodibenzo[b,f]thiepin-10-yloxy)-1-ethyl)-3-piperidine- carboxylic acid; 1-(2-(8-Methylthio-10,11-dihydrodibenzo[b,f]thiepin-10-yloxy)-1-ethyl)-3-piperidine- carboxylic acid; (R)-1-(2-(10,11-Dihydrodibenzo[b,floxepin-10-yloxy)ethyl)-3-piperidinecarboxylic acid; (R)-1-(2-(2-Chloro-10,11-dihydrodibenzo[b,f]thiepin-10-ylsulfanyl)ethyl)-3- piperidinecarboxylic acid; (R)-1-(11 H-Dibenz[b,f][1 ,4]oxathiepin-11-ylmethyl)-3-piperidinecarboxylic acid; (R)-1-(2-(2-Chloro-7-fluoro-10,11-dihydrodibenzo[b,f]thiepin-10-yloxy)ethyl)-3- piperidinecarboxylic acid; (R)-1-(2-(2,4-Dichloro-10,11-dihydrodibenzo[b,f]thiepin-10-yloxy)ethyl)-3-piperidinecarboxylic acid, or a pharmaceutically acceptable salt thereof.

32. The use according to anyone of the claims 1-3 wherein in formula Id Rid and R²d independently are hydrogen, halogen, trifluoromethyl, hydroxy, C₁₋₆-alkyl or C,6- alkoxy; and Xd is —O—,—S— or —S(═O)—; and rd is 0,1,2,3,4,5,6,7,8,9 or 10; and Zd is selected from 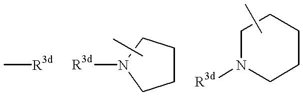
wherein R3d is —(CH₂)mdOH or —(CH₂)pdCOR⁴d wherein md and pd independently are 0, 1, 2, 3 or 4 and R⁴d is OH, NH₂, NHOH or C₁₋₆-alkoxy; or a pharmaceutically acceptable salt thereof.

33. The use according to anyone of the claims 1-3 and 32 wherein the compound is selected from the following: 4-(1,3,4,14b-Tetrahydro-2H-dibenzo[b,f]pyrazino[1,2-d][1,4]oxazepin-2-yl)-butanoic acid; 4-(1,3,4,1 4b-Tetrahydro-2H-dibenzo[b,f]pyrazino[1 ,2-d][1 ,4]thiazepin-2-yl)-butanoic acid, or a pharmaceutically acceptable salt thereof.

34. The use according to any of the claims 1-33 wherein the pharmaceutical composition is in a form suitable for oral administration.

35. A method of treating an indication related to angiogenesis comprising administering to a subject in need thereof an effective amount of a compound according to any of the claims 1-33.

36. A method according to claim 35 wherein angiogenesis is related to cancer.

37. A method according to claim 35 wherein angiogenesis is related to ocular neovascularization.

38. Any novel feature or combination of features described herein.
